(19) United States
(12) Patent Application Publication
(10) Pub. No.: US 2021/0254049 A1
(43) Pub. Date: Aug. 19, 2021

(54) DIRECTED CELL FATE SPECIFICATION AND TARGETED MATURATION

(71) Applicant: CELLINO BIOTECH, INC., Cambridge, MA (US)

(72) Inventors: Stan Wang, Cambridge, MA (US); Edroaldo Lummertz Da Rocha, Cambridge, MA (US); Suvi Aivio, Arlington, MA (US)

(21) Appl. No.: 17/049,243

(22) PCT Filed: Apr. 19, 2019
(86) PCT No.: PCT/US2019/028352

§ 371 Date: Oct. 20, 2020

Related U.S. Application Data
(60) Provisional application No. 62/739,027, filed on Sep. 28, 2018.
(60) Provisional application No. 62/660,577, filed on Apr. 20, 2018.

(51) Int. Cl. C12N 15/10; C12N 5/0781

(52) U.S. Cl. CPC C12N 15/1079; C12N 5/0635; C12N 2503/02; C12N 2510/00; C12N 2506/45

(57) ABSTRACT

Methods for identifying targets involved in cell differentiation, for instance particular loci of a human genome. To identify such targets, for example, complexes that each include a catalytically-deactivated DNA binding protein, a guide RNA that guides the complex, and one or more effector domains may be introduced into stem cells to cause at least one of the stem cells to differentiate into a target phenotype. The guide RNAs present in cells demonstrating the target phenotype may be identified and a nucleic acid sequence of each identified guide RNA may be correlated to loci of a genome to identify targets involved in directing cell differentiation to the target phenotype. These methods may be used for directed cell fate specification in stem cells, such as induced pluripotent stem cells, to produce synthetic cells with a desired target phenotype.

### CROSS-REFERENCE TO RELATED APPLICATIONS

This application claims priority to and the benefit of U.S. Provisional Application No. 62/739,027 filed Sep. 28, 2018 and U.S. Provisional Application No. 62/660,577 filed Apr. 20, 2018, the contents of each of which are incorporated by reference herein in their entirety.

### TECHNICAL FIELD

The invention relates to directed cell fate specification and screening methods.

### BACKGROUND

Stem cells are cells that are characterized by the ability to multiply indefinitely and the ability to develop into many different cell types. Some stem cells even have the potential to develop into any specific cell type. Stem cells are potentially useful in medicine as a source of cells to supplement or replace cells lost to disease. They also have the potential to be differentiated into specific cell types to be used in the production of certain therapeutics of biological origin, such as insulin or clotting factors.

Efforts to differentiate stem cells to specific cell types for use in research or medicine are met with limited success due to the complexity and unpredictability of the biological processes involved. For a few certain cell types, it is understood that certain combinations of growth or transcription factors may be delivered under controlled culture conditions to allow the stem cells to differentiate. However, only a few differentiation pathways have been studied. Due to the unpredictability involved, those few pathways that have been studied do not provide general guidance for differentiating stem cells into various desired specific cell types.

Additionally, the present technology in the field does not allow for their guided maturation to terminally differentiated or fully functional cell types with a minimal set of targeted genes and associated effectors for modulating their expression. The present approach is comprised of manual trial by error, which is time consuming and inefficient. Therefore, there is a need in the art for a more generalized and automated approach to utilizing stem cells to produce specified mature cells, to aid in guiding experiments in biological research and further developments in regenerative medicine.

### SUMMARY

The invention provides methods for screening stem cells to identify genomic loci where transcription can be activated or repressed to differentiate stem cells to a desired cell type. Cas proteins that bind genomic DNA in a sequence specific manner and regulate transcription are presented to stem cells in a high throughput screen. When one of the Cas proteins causes differentiation into the desired cell type, the binding sequence is identified as a genomic locus at which transcriptional regulation can be employed to differentiate stem cells into the desired cell type. The invention further provides methods of differentiating stem cells to desired cell types using Cas proteins and guide RNAs that target the Cas proteins to the identified loci to participate in transcriptional regulation.

In particular, stem cells are provided with catalytically-inactive Cas (dCas) endonuclease proteins linked to effector domains that participate in transcriptional regulation. Guide RNAs are introduced that guide the dCas proteins to their respective genomic targets within the stem cells. Where a dCas protein then binds to a target that is a promoter for a gene involved in differentiation, the linked effector domain participates in the up- or down-regulation of transcription of the gene. For a stem cell that differentiates to the desired cell type, the targeting portion of the associated guide RNA is then understood to be complementary to the genomic locus, or promoter, that can be targeted to cause stem cells to differentiate to the desired cell type. The guide RNAs and associated dCas protein linked to effector domains that are so discovered to differentiate stem cells may then be used provided or used to differentiate stem cells into the desired cell type going forward.

Methods may include selecting an abundance of guide RNAs that target promotor areas of genes suspected to be involved in differentiating cells to a specified differentiated cell type, and then using the methods to identify, from among the abundance (e.g., hundreds or thousands) of the guide RNAs and effective set (e.g., 1 to 40 per gene for 1 to about any number of different genes associated with the specified differentiated cell type) of guide RNAs, in which an effective set is a set of one to a few dozen guide RNAs that can be delivered with a CRISPRa/i protein to effectively differentiate stem cells into the specified differentiated cell type. Selecting that initial abundance of guide RNAs can include a process that includes (1) first a literature search to identify genes suspected to be involved in differentiating cells to the specified differentiated cell type followed by (2) and analysis or search such as a genomic database search (e.g., in GenBank or Ensembl) to identify suitable guide RNA targets (e.g., unique or nearly-unique 20 base stretches, adjacent to a protospacer adjacent motif, within putative promoter regions of genes identified in step 1. The analysis for step 2 may include implementation of software/algorithms to predict the activity of different gRNA sequences within a promoter sequence.

Proteins originally found in bacteria in association with clustered, regularly interspersed palindromic repeats (CRISPR) have been dubbed CRISPR-associated (Cas) proteins. Of those, Cas9 was initially identified as an RNA-guided endonuclease that complexes with both a trans-activating RNA (tracrRNA) and a CRISPR-RNA (crRNA), and is guided by the crRNA to an approximately 20 base target within one strand of double-stranded DNA (dsDNA) that is complementary to a corresponding portion of the crRNA, after which the Cas9 endonuclease creates a double-stranded break in the dsDNA. Cas9 endonuclease is one example among a number of homologous Cas endonucleases that similarly function as RNA-guided, sequence-specific endonucleases. Some variants of Cas endonucleases in which an active site is modified by, for example, an amino acid substitution, have been found to be catalytically inactive, or “dead”, Cas (dCas) proteins and function as RNA-guided DNA-binding proteins. Cas endonucleases and dCas proteins are understood to work with tracrRNA and crRNA or with a single guide RNA (sgRNA) oligonucleotide that includes both the tracrRNA and the crRNA portions and, as used herein, “guide RNA” includes any suitable combination of one or more RNA oligonucleotides that will form a ribonucleoprotein (RNP) complex with a Cas protein or dCas protein and guide the RNP to a target of the guide RNA. The guide RNAs typically include a targeting portion of about 20 bases which will hybridize to a complementary target in dsDNA, when that target is adjacent a short motif dubbed the protospacer-adjacent motif (PAM), to thereby bind the RNP to the dsDNA. When dCas protein is linked to an effector domain and complexed with guide RNA, the resultant complex can upregulate or downregulate transcription. When the target of the guide RNA is within a promoter, the linked effector domain can recruit RNA polymerase or other transcription factors that ultimately recruit the RNA polymerase, which RNA polymerase then transcribes the downstream gene into a primary transcript such as a messenger RNA (mRNA). Such a use of dCas protein to modulate transcription may be exploited to assay for which guide RNAs initiate transcription that results in a particular cellular phenotype and, by mapping a target of those guide RNAs to a particular locus in a reference genome, to identifier promoters at which to regulate transcription to direct a cell to the particular cellular phenotype.

Thus, methods of the disclosure include introducing RNPs that include dCas linked to an effector domain and complexed with a guide RNA into stem cells to differentiate the stem cells into a target phenotype. Cells demonstrating the desired target phenotype may then be selected and optionally enriched or cultured for further analysis. The effector domains may cause various activities in the stem cells to cause cell differentiation, for example, an activating activity, an inhibiting activity, or recruiting activity where co-activating or co-inhibiting proteins are recruited to the complex. The stem cells may be any stem cells, for example, induced pluripotent stem cells, pluripotent stem cells, totipotent stem cells, or multipotent stem cells.

Once cells with the target phenotype are selected, the gRNAs targeting loci of the genome may be identified, thereby identifying at least one of the gRNAs or effector domains that caused at least one of the stem cells to differentiate into a target phenotype. As such, the disclosed methods allow the identification and characterization of targets involved in causing cell differentiation. These methods may be used to identify targets that can be activated, inhibited, or altered to produce cells of any target phenotype from any starting cell type. Through the application of the disclosed methods, stem cells can be transformed into specific cell types that my serve as, or may produce, useful therapeutic agents for the treatment of diseases.

In certain aspects, the disclosure provides screening methods for identifying targets involved in cell differentiation. Methods include introducing into each of a plurality of stem cells a dCas protein linked to a transcription regulator and one or more guide RNAs, isolating—from the plurality of stem cells—a viable cell that contains the dCas protein linked to the transcription regulator and at least one of the guide RNAs, and measuring gene expression in the viable cell or progeny thereof. A change in gene expression in the viable cell or progeny thereof is correlated with one or more targets of the guide RNAs in the viable cell or progeny thereof.

The transcription regulator under guidance of the dCas protein and one or more guide RNAs may initiate differentiation of one of the plurality of stem cells into the viable cell or progeny thereof such that correlating the change in gene expression with the targets of the guide RNAs identifies loci to target by CRISPRa and/or CRISPRi to differentiate pluripotent stem cells into a target cell type.

Certain embodiments include a combinatorial approach in which CRISPRa/i regulates expression of some factors in combination with the direct introduction or otherwise induced expression of other factors. Methods may include initiating expression of, or introducing, one or more additional gene products to promote differentiation of the one of the plurality of stem cells into the viable cell or progeny thereof. Expression of at least one of the additional gene products may be initiated by introducing a corresponding gene using, e.g., a PiggyBac transposon; introducing a corresponding gene via a plasmid or viral vector; or introducing an mRNA encoding the gene product. The additional gene products may be introduced as a protein to the one of the plurality of stem cells. In an illustrative embodiment, the gene product is a transcription factor and the transcription factor and the transcription regulator under guidance of the dCas protein and one or more guide RNAs results in differentiation of the one of the plurality of stem cells into a beta islet cell.

Some embodiments involve a temporal sequence of CRISPRa/i to differentiate cells. Guide RNAs (e.g., with the dCas protein linked to the regulator) may be introduced into at least one of the plurality of stem cells in a temporal sequence. The temporal sequence may include the introduction of a first set of one or more guide RNAs during a first period comprising one or more hours or days followed by introduction a second set of one or more guide RNAs during a second period comprising one or more hours or days. Optionally, the first set of one or more guide RNAs and the second set of one or more guide RNAs comprise wholly different guide RNAs and/or the first period and the second period do or do not overlap in time. In some embodiments, CRISPRa/i is used against a first set of targets during the first period, the first period comprising at least two days, and using CRISPRa/i against a second set of targets during the second period to differentiate the one of the plurality of stem cells into a glucose-responsive insulin-secreting beta cell.

In some embodiments of the methods, isolating the viable cell includes selecting a cell that exhibits a desired trait. Selecting the cell that exhibits the desired trait may include staining the plurality of stem cells with a marker for the desired trait, and sorting the cells using, for example, a fluorescence-activated cell sorting instrument, a magnetic bead-based purification, others, or a combination thereof. In some embodiments, the desired trait includes a specified differentiated cell type and the marker includes a protein expressed by the differentiated cell type. The desired trait may include a beta cell phenotype, and marker one or more of the presence of C-peptide, Insulin, Chromogranin A, and Nkx6.1, and the absence of Glucagon and Somatostatin.

In some embodiments, measuring gene expression in the viable cell or progeny thereof includes one or more of: quantifying expression levels via RNA-Seq; and evaluating DNA-protein interaction via chromatin immunoprecipitation and DNA sequencing (ChIP-seq). The methods may include determining fold-change in expression level of a transcript associated with the marker by normalizing read counts from the measuring against control read counts. In certain embodiments, the guide RNAs are barcoded, and the method further comprises using a computer system to analyze sequence data to determine the fold-change for the transcript and correlate, using barcode sequences in the sequence data, the fold-change for the transcript with the one or more targets of the guide RNAs in the viable stem cell.

In preferred embodiments, introducing the dCas protein linked to the transcription regulator into the stem cells includes delivering to the stem cells a vector that encodes a fusion protein comprising the dCas protein and the transcription regulator. The vector may include a viral vector, a plasmid, or transposable element. Optionally, the vector further has a selection marker, and the method includes selecting for cells transformed by the vector prior to the isolating step. The cells may be selected for transformation by the vector prior to introducing the one or more guide RNAs.

Embodiments of the methods include distributing the plurality of stem cells into reaction vessels such that each reaction vessel receives, on average, between 0 and 2 of the stem cells. Introducing the one or more guide RNAs may include obtaining guide RNAs that have targeting portions that map to promoter regions of genes associated with a desired phenotype or trait, and delivering to each reaction vessel guide RNAs that target either one or a plurality of genes associated with the desired phenotype or trait. For each gene that is targeted, between one and 40 distinct guide RNAs may be delivered (e.g., preferably about 10 to 30). For each guide RNA that is delivered, between about 1 and about 20 copies of the guide RNA may be delivered.

Isolating the viable stem cell may include selecting a cell that exhibits a specified differentiated cell type, and the guide RNAs may have targeting portions that map to promoter regions of genes associated with the differentiated cell type. The method may thus include identifying promoter regions of genes to target for transcription regulation using a dCas protein linked to a transcription regulator to differentiate stem cells to the specified differentiated cell type.

In certain embodiments, methods include identifying the one or more targets of the guide RNAs by sequencing at least a portion of the guide RNAs to produce sequence reads and mapping the sequence reads to a reference to identify genomic loci targeted by the guide RNAs. The viable cell or progeny thereof may be differentiated cells of a specific cell type. In some embodiments, the method includes identifying the differentiated cells by sequencing nucleic acid from the differentiated cells. The nucleic acid may include gene transcripts resulting from transcriptional activation by the dCas protein linked to the transcription regulator. The guide RNAs and gene transcripts may be sequenced via RNA-Seq using a next-generation sequencing platform.

In some embodiments, the methods include determining a network of targets involved in directing cell differentiation by identifying a plurality of targets involved in directing the stem cells to a target phenotype.

The transcription regulator may include one or more effector domains that recruit coactivator or corepressor proteins to the dCas protein-linked transcription regulator.

The methods include (1) RNP embodiments, (2) mRNA embodiments, and/or (3) DNA embodiments. In the RNP embodiments, introducing the dCas proteins and delivering the guide RNAs are done as a single step by providing the stem cell with a ribonucleoprotein (RNP) comprising the dCas protein linked to the transcription regulator and complexed with one of the guide RNAs. In the mRNA embodiments, introducing the dCas proteins and delivering the guide RNAs includes providing the each of the stem cells with (i) an mRNA encoding a fusion protein that includes the dCas protein and the transcription regulator and (ii) at least one of the guide RNAs. In DNA embodiments, introducing the dCas proteins includes delivering a vector comprising a gene for a fusion protein that includes the dCas protein and the transcription regulator.

In certain aspects, the invention provides a screening method for identifying targets involved in cell differentiation. The method includes obtaining a plurality of stem cells and differentiating a stem cell of the plurality to a desired phenotype by introducing into the stem cell a dCas protein linked to a transcription regulator and at least one guide RNA. The method further includes identifying a target of the at least one guide RNA in the differentiated cell, thereby determining one or more transcriptional regulation targets for differentiating stem cells to the desired phenotype. The transcription regulator may include one or more effector domains that recruit coactivator or corepressor proteins to the dCas protein-linked transcription regulator.

Identifying the target of the at least one guide RNA in the differentiated cell may be done by sequencing at least a portion of the at least one guide RNAs to produce sequence reads and mapping the sequence reads to a reference to identify genomic loci targeted by the guide RNAs.

The method may also include identifying the differentiated cell by sequencing nucleic acid from the differentiated cells. In some embodiments, the nucleic acid that is sequenced includes gene transcripts resulting from transcriptional activation by the dCas protein linked to the transcription regulator. The guide RNAs and the gene transcripts may both be sequenced (e.g., via RNA-Seq) using a next-generation sequencing platform.

Introducing the dCas proteins and delivering the guide RNAs may be done as a single step by providing the stem cell with a ribonucleoprotein (RNP) comprising the dCas protein linked to the transcription regulator and complexed with the guide RNA. The stem cells may be stimulated to take up the formed RNP using a technique such as electroporation, nanoparticle transfection, or preferably laser excitation of plasmonic substrates. Optionally, introducing the dCas proteins and delivering the guide RNAs includes providing the stem cells with: an mRNA encoding a fusion protein that includes the dCas protein and the transcription regulator; and at least one of the guide RNAs. In some embodiments, introducing the dCas proteins includes delivering a vector comprising a gene for a fusion protein that includes the dCas protein and the transcription regulator. The vector (e.g., a plasmid or viral vector) may be constitutively expressed in the stem cells. The vectors may be introduced into the stem cells by transfection or transduction.

Methods may include determining a network of targets involved in directing cell differentiation by identifying a plurality of targets involved in directing the stem cells to the target phenotype. In some embodiments, the stem cells comprise induced pluripotent stem cells.

In one aspect of the invention, a screening method for identifying targets involved in cell differentiation includes introducing complexes with gRNAs and one or more effector domains into stem cells and identifying at least one of the guide RNAs or effector domains that caused at least one of the stem cells to differentiate into a target phenotype. The starting stem cells may be of any cell type, including totipotent stem cells, pluripotent stem cells, and multipotent stem cells. Preferably, embodiments may use induced pluripotent stem cells (iPS cells or iPSC), which are pluripotent stem cells generated from adult cells. Methods for generating iPS cells from adult stem cells through the introduction of iPS reprogramming factors are known in the art. The iPS cells may of any origin, for instance, human iPS cells.

The complexes introduced can have various activities in the stem cells to cause cell differentiation into the target phenotype, for example, the activity may activate or repress genes that encode proteins involved in cell differentiation or may recruit coactivator or corepressor proteins to the complex to cause an activating or inhibiting activity. The target phenotype may be any target phenotype. For example, the target phenotype may be an adult cell for an external layer of the body, such as a skin cell or a neuron cell. Alternatively, the target phenotype may be an adult cell of an internal layer of the body, such as a lung cell, a thyroid cell, or a pancreatic cell. Further, the target phenotype may be an adult cell of a middle layer of the body, such as a cardiac muscle cell, a skeletal muscle cell, a smooth muscle cell in the gut, a tubule cell in the kidney, or a red blood cell. In any event, the targets involved in causing a stem cell to differentiate into a specialized cell type should be known and their interactions understood. With such knowledge, stem cell treatments will benefit from the ability to properly and intentionally direct cell fate—the inducement of stem cells to differentiate into the desired target phenotype. Additionally, methods of the disclosure may be useful for the production of artificial cells with synthetic but desired characteristics.

Certain methods are known to activate or repress genes, but despite advances, no prior generalizable methods or systems exist for the identification or prediction of combinations of factors via a screening method that would allow production of synthetic cells with any desired target phenotype.

According to methods of the invention, the complexes introduced into stem cells may include a DNA binding protein that is guided by a gRNA to particular loci of the genome. In various embodiments, the complex includes a Clustered Regularly Interspaced Short Palindromic Repeats (CRISPR)-associated protein (Cas protein). Any Cas protein forming a complex with the gRNAs and the effector domains may be used, although in preferable embodiments, the Cas protein is a catalytically-deactivated Cas protein (dCas).

A Cas protein is a type of DNA binding protein that can form a complex with and be guided by a gRNA. One example of a Cas protein is a Cas9 protein, which is an RNA-guided DNA binding endonuclease derived from S. pyogenes. Cas9 may by guided to a location substantially complementary to a sequence of the gRNA and enzymatically cleave the DNA at a location adjacent to a sequence known as protospacer adjacent motif (PAM) (e.g., NGG, where N is any nucleotide and G is guanine nucleotide). The changes produced by a Cas9 are permanent to a genome. In contrast, a dCas9 is a catalytically-deactivated Cas9 protein, which retains its DNA binding ability but not its endonuclease DNA cleaving ability. Complexes including dCas proteins can be targeted to various genomic locations via gRNAs to bind specific locations of DNA. The changes produced by dCas9 are reversible.

When dCas proteins and gRNAs are complexed with one or more effector domains, the resulting complexes use the gRNAs to target substantially complementary sequences of a genome. The genome may be any genome, for example, a human genome. When the gRNA hybridizes to this sequence, the dCas binds the DNA and allows the effector domains to cause an activity. For example, when one or more effector domains act as an activator, complexes can target specific loci via gRNAs and activate genes. The effector domains may also recruit activators or other co-activating molecules to the site to cause an activating activity. This activating activity may be referred to as CRISPRa. Similarly, in another example, when one or more effector domains act as an inhibitor, complexes can target specific loci via gRNAs and inhibit (i.e., repress) genes. In other examples, the effector domains recruit inhibitors or other co-inhibiting molecules to the site to cause an inhibiting activity. This inhibiting activity may be referred to as CRISPRi. Embodiments of the disclosed methods may use either or both CRISPRa and CRISPRi to activate or inhibit genes involved in cell differentiation. As such, the disclosed methods may be referred to as CRISPRa/i methods.

When, the complexes are introduced into the stem cells, and at least one of the stem cells differentiates into a target phenotype, the guiding sequences of the gRNAs that guided the complexes may be used to identify loci involved in causing cell differentiation into that target phenotype. To perform the disclosed methods, pre-designed gRNAs may be commercially obtained or designed such that the sequences of each are known. Alternatively, in one embodiment, the method further includes sequencing the guide RNAs to produce sequence reads and mapping the sequence reads to loci of a genome, thereby identifying one or more targets involved in causing differentiation into the target phenotype. For example, sequencing may be by single-cell next-generation sequencing (NGS).

Depending on whether complexes were designed to cause activating activities or inhibiting activities, methods of the invention indicate whether the gRNA-targeted genes should be transcriptionally turned “on” (activated) or “off” (inhibited) to induce cells to differentiate into the target phenotype. Often, combinations of gene activation and/or repression across multiple genes are involved in and required to direct cell fate (i.e., multiple genes must be activated and/or inhibited in specific ordered combinations to cause a cell to differentiate into the desired adult cell type). Thus, in certain embodiments, methods further include determining combinations of factors interrelated in directing cell differentiation by identifying a plurality of targets involved in directing the stem cells to the target phenotype.

According to the methods of the invention, the complexes may be introduced into the stem cells in any suitable manner. In one embodiment, introducing complexes includes co-introducing gRNAs and an mRNA encoding dCas. In one example, the dCas encoded by the mRNA comprises the one or more effector domains. In this example, the effector domain is a domain that recruits coactivator or corepressor proteins to the complex. In certain embodiments, the dCas is constitutively expressed in the stem cells. In other embodiments, the complexes are introduced into the stem cells by transfection or transduction.

In another aspect of the invention, a method for identifying targets involved in cell differentiation includes introducing complexes comprising guide RNAs and one or more effector domains into stem cells, identifying at least one of the guide RNAs or effector domains that caused at least one of the stem cells to differentiate into a target phenotype, and correlating a nucleic acid sequence of the guide RNAs to loci of a genome, thereby identifying one or more targets involved in causing cell differentiation into the target phenotype.

An embodiment of the invention is directed to methods of identification and characterization of insulin-producing beta-like cells, as well as monohornonal and polyhormonal discrimination. In an example, cellular RNA is collected and analyzed, such as by qRT-PCR, microarray, and/or next generation sequencing, for differential expression of beta cell-specifying genes, insulin (INS) genes, and glucagon (GCG) genes. In an example, cells are fixed and stained for expression of insulin (c-peptide), glucagon, chromogranin A, or any combination thereof. In another example, cells are stimulated to secrete insulin via escalating doses of glucose-supplemented media. The analytical techniques are used to determine which methods derive reproducible beta-like cell populations that optimize the quantity and purity of beta cells from stem cells. In certain embodiments, laser-activated intracellular delivery of CRISPR-Cas systems is used for genome engineering and altering gene expression in induced pluripotent stem cells (iPSCs).

After analysis, methods of the invention further comprise validating the reproducible beta-like cell populations derived for in vivo functionality through transplantation in mouse models. In an example, beta-like cells are transplanted into normoglycemic mice, which undergo periodic fasting blood glucose and glucose challenge testing to elicit insulin responsiveness, followed by sacrifice and explant analysis for maintenance of cell identity at the end of the animal trial period. In another example, beta-like cells are transplanted into hyperglycemic/non-obese diabetic (NOD) mice and the process described above is followed. Beta cell supplementation in hyperglycemic mice likely contributes to glycemic normalization through glucose-responsive insulin production, resulting in potential extension of life.

Any suitable approach may be used to modulate the activity of the genes. In an example, an approach for modulating activity is direct expression by factor introduction. Another example approach is over-/under-expression by CRISPR activators/inhibitors (CRISPRa/i). In an embodiment, the present invention provides approaches and techniques at the DNA level used to achieve the direct expression of the desired gene. In another embodiment, approaches and techniques are provided at the RNA and/or protein level used to achieve the direct expression of the desired gene. In certain embodiments, laser-activated intracellular delivery of CRISPR-Cas systems is used for genome engineering and altering gene expression in induced pluripotent stem cells (iPSCs). In an embodiment, in order to drive the overexpression or inhibition of endogenous loci, CRISPRa/i is used with one or more single guide RNAs (sgRNAs) that target within −300 to +0 base pairs of the transcription start site (TSS) per target gene in stable cell lines or ribonucleoprotein (RNP) complexes.

In an example, stable cell lines expressing the dCas9-VPR, or other suitable CRISPRa constructs, are generated via lentiviral or piggyBac incorporation into the genome with constitutive or drug-inducible promoters, along with fluorescent and/or drug selection markers. In this instance, sgRNAs may be delivered to these stable cell lines with nanoparticle-based transfection (e.g. lipofection), electroporation (e.g. nucleofection), laser-activation of substrates (i.e. NanoLaze), or other physical delivery methods. Repetitive delivery with the same or different sgRNA permutations on the same or subsequent days may be necessary to yield differentiation.

Certain aspects of the invention provide systems and methods for directing cell fate. Systems and methods of the invention allow the use of minimal target/effector combinations to direct differentiation of stem cells. The invention identifies genomic targets that promote differentiation of a desired cell type and optimizes the cellular differentiation process by identifying a minimal number of targets and a corresponding CRISPR-associated guide RNA effector sequences. As described below, selected genomic targets are exposed to Cas/guide RNA complexes and are characterized to assess progress toward differentiation into a desired cell type. Cycles of exposure to selected minimum numbers of effectors can continue as necessary until an endpoint is achieved.

Methods and systems for directing cell fate include selecting a minimal number of genomic targets responsible for directing cell differentiation into a desired cell type. A minimum number of guide RNA sequences corresponding to each of the selected genes are identified. The guide RNAs form a complex with a Cas protein, and the Cas-gRNA complex is introduced into each a plurality of stem cells to promote cell differentiation to a desired cell type. Cells are then assessed to determine which of them has progressed toward target cell type. Assessment may be carried out by comparing identified traits of the targeted cells to specific traits characteristic of the differentiated cell. If a desired cell end point is not achieved in the first cycle, the cycle may be repeated with a minimal number of genes thought to be associated with the desired differentiated cell type. In some embodiments, the genes identified in the first cycle, may also be identified in subsequent cycles. In other embodiments, the desired cell type may be achieved after the first cycle. In yet other embodiments, the cycle may be repeated to further enhance a phenotype of the desired cell type.

To identify genomic targets and sequences of corresponding guide RNAs, aspects of the invention include analysis of data from a plurality of data sources. Preferred data sources include, but are not limited to, publications, public data sets (e.g., gene expression data sets), cell type characterization profiles, the output from systems of algorithms, and internal data sets, including laboratory results, of, for example scRNA-seq (single-cell RNA sequencing) expression data obtained from the differentiated cells produced by methods of the invention. In other embodiments, identifying initial minimum guide RNA sequences includes (1) a literature search to identify genes suspected to be involved in differentiating cells to a specified differentiated cell type and (2) searching, such as a genomic database search (e.g., in GenBank or Ensembl), to identify suitable guide RNA targets (e.g., unique or nearly-unique 20 base stretches, adjacent to a protospacer adjacent motif, within putative promoter regions of genes identified in step 1). Methods may also include (3) analysis of the data to identify a temporal sequence of gene expression to direct cell fate specification of the desired cell type.

In another embodiment, methods of the invention may further include implementation of software/algorithms to predict the activity of different gRNA sequences within a promoter sequence. Such methods of analysis include the identification of at least one gRNA per target gene that maps to the promoter region of the gene to optimally activate the gene. If the cell type is not achieved, steps 1-3 are repeated until the cell type is achieved.

Guide RNAs target promotor regions of identified genes that are known or suspected to be involved in differentiation of a selected cell type. Preferred gRNA typically includes a targeting portion of about 20 bases that hybridizes to a complementary target in double stranded DNA (dsDNA) when that target is adjacent a short motif dubbed the protospacer-adjacent motif (PAM). Identifying a minimum number of guide RNAs may include introducing into each of a plurality of cells a Cas protein and a guide RNA complex to produce a viable cell, or progeny thereof, measuring gene expression of the target in the viable cell to identify a minimum number of guide RNAs causing optimal gene expression of the target gene. Gene expression can be analyzed by methods known in the art, e.g., RT-qPCR. In another embodiment, the minimum set of guide RNAs are identified by bioinformatics analysis of the data. The guide RNAs can be a set of one to a ten guide RNAs that can be complexed with a Cas protein and delivered to a stem cell to effectively target the gene to differentiate cells into the desired cell type. In other embodiments, an effective set of gRNAs per gene may be a pool of 4-5 gRNAs. In yet another embodiment, an effective set of gRNAs may be 2-4 gRNAs per gene.

Methods of the invention may include stem cells, which may be of any cell type, including totipotent stem cells, pluripotent stem cells, and multipotent stem cells. Preferably, embodiments may use induced pluripotent stem cells (iPS cells or iPSC), which are pluripotent stem cells generated from adult cells. Methods for generating iPS cells from adult stem cells through the introduction of iPS reprogramming factors are known in the art. The iPS cells may of any origin, for instance, human iPS cells.

In certain aspects of the invention, Cas proteins are complexed with the minimum set of guide RNAs and introduced into to stem cells to target the identified genes so as to differentiate the stem cell to a desired cell type. Proteins originally found in bacteria in association with clustered, regularly interspersed palindromic repeats (CRISPR) have been termed CRISPR-associated (Cas) proteins. Cas9 endonuclease is one example of many homologous Cas endonucleases that function as RNA-guided endonucleases. Cas endonucleases can be complexed with both a trans-activating RNA (tracrRNA) and a CRISPR-RNA (crRNA), and is guided by the crRNA to an approximately 20 base target within one strand of dsDNA that is complementary to a corresponding portion of the crRNA, after which the Cas endonuclease creates a double-stranded break in the dsDNA. Variants of Cas endonucleases in which an active site is modified by, for example, an amino acid substitution, may be catalytically inactive, or “dead” Cas (dCas) proteins and function as RNA-guided DNA-binding proteins. Cas endonucleases and dCas proteins are understood to work with tracrRNA and crRNA, or with a single guide RNA (sgRNA) oligonucleotide that includes both the tracrRNA and the crRNA portions, and, as used herein, “guide RNA” or “gRNA” includes any suitable combination of one or more RNA oligonucleotides that will form a ribonucleoprotein (RNP) complex with a Cas protein or dCas protein and guide the RNP to a target of the guide RNA. When dCas protein is linked to an effector domain and complexed with guide RNA, the complex can upregulate or downregulate transcription. In other aspects of the invention, the stem cells are provided with dCas ribonucleoproteins (RNPs) linked to effector domains that participate in transcriptional regulation. When the target of the guide RNA is within a promoter, the linked effector domain can recruit RNA polymerase or other transcription factors that ultimately recruit the RNA polymerase, which RNA polymerase then transcribes the downstream gene into a primary transcript such as a messenger RNA (mRNA).

The guide RNAs (gRNAs) identified by methods of the invention are thus complexed with Cas proteins and guide the Cas proteins to their respective genomic targets within the stem cells. The gRNAs and associated Cas protein link to domains of genes identified by methods of the invention as a minimum gene necessary to differentiate the cells into the desired cell type. The methods of differentiating a cell to a desired cell type, or subtype using Cas proteins and minimum guide RNAs that target the Cas proteins to the identified minimum target genes to participate in transcriptional regulation of the cell to the desired cell type, or subtype. In yet another embodiment of the invention, the Cas protein, is a dCas protein and is linked to an effector, for example, a transcription regulator.

The complexes introduced can have various activities in the stem cells to cause cell differentiation into a desired cell type. For example, the activity may activate or repress genes that encode proteins involved in cell differentiation or may recruit coactivator or corepressor proteins to the complex to cause an activating or inhibiting activity. The desired cell type may be any cell type or subtype and may have a specific phenotype, be at any stage of maturity or state of differentiation. For example, the desired cell type may be an adult cell, an intermediary cell, an immature cell, or any cell type in between. The desired cell type may be for an external layer of the body, such as a skin cell. Alternatively, the desired cell type may be an adult cell of an internal layer of the body, such as a lung cell, a thyroid cell, or a pancreatic cell. Further, the desired cell type may be an adult cell of a middle layer of the body, such as a cardiac muscle cell, a skeletal muscle cell, a smooth muscle cell in the gut, a tubule cell in the kidney, or a red blood cell. Furthermore, the desired cell type may be an adult cell of the nervous system. In other embodiments, the desired cell type may be a target phenotype. For example, the target phenotype may be a dopaminergic neuron. In any event, the targets involved in causing a stem cell to differentiate into a specialized cell type should be known and their interactions understood.

In some embodiments, methods include identifying a temporal sequence of gene expression to differentiate the cells to the cell type. gRNAs (with or without the dCas protein linked to the regulator) may be introduced into at least one of the plurality of cells in a temporal sequence. The temporal sequence may include the introduction of a first set of one or more guide RNAs during a first period comprising one or more hours or days, followed by introduction a second set of one or more guide RNAs during a second period comprising one or more hours or days. Optionally, the first set of one or more guide RNAs and the second set of one or more guide RNAs comprise wholly different guide RNAs and/or the first period and the second period do or do not overlap in time. In some embodiments, CRISPRa/i is used against a first set of targets during the first period, the first period comprising at least two days, and using CRISPRa/i against a second set of targets during the second period to differentiate the one of the plurality of stem cells into the desired cell type. In a preferred embodiment, the desired cell type is a dopaminergic neuron.

Aspects of the invention may include identifying the cell type of the differentiated cells. Cell types are identified by specific cell traits that have been previously identified as characteristic of a certain cell type. Cell traits may include cell morphology, chromosome analysis, DNA analysis, protein expression, RNA expression, enzyme activity, cell-surface markers, or a combination thereof. Each of the differentiated cells produced by methods of the invention may be characterized by cell traits. Characterizing the cells may include identifying cell traits by staining the cells with a marker for the desired characteristic, and sorting the cells using, for example, a fluorescence-activated cell sorting instrument, a magnetic bead-based purification, others, or a combination thereof. In another embodiment, characterizing the cells may include identifying cell traits by measuring gene expression in the cell or progeny thereof. Gene expression includes one or more of: quantifying expression levels via RNA-Sequencing; measuring gene expression via single-cell RNA sequencing; or evaluating DNA-protein interaction via chromatin immunoprecipitation and DNA sequencing (ChIP-seq). The methods may include determining fold-change in expression level of a transcript associated with a marker of a specific cell type by normalizing read counts from the measuring against control read counts. The methods may also include comparing transcriptomes of individual cells to assess transcriptional similarities and differences between the cells. The cell type of each of the cells may be determined by comparing the identified traits of each of the cells to the known traits of a cell type. The methods may also include identifying cell type by comparing transcriptomes of the cells to assess transcriptional similarities and differences between the cells and may include clustering like cells. In an embodiment, the desired trait includes a specified differentiated cell type and the marker includes a protein expressed by the differentiated cell type. In another embodiment, the desired trait may be a neuronal phenotype, and marker one or more of the presence of beta III tubulin and DAPI and the absence of Oct4. In a preferred embodiment, the desired trait may include an inducible neuron phenotype, and the marker the presence of beta III tubulin.

Aspects of the invention provide systems and methods to collect, analyze, and store data sets to provide a user with cell type data. The cell type data may be any type of data described herein, for example genes involved in differentiation of a cell type and their respective genetic sequences, guide RNA sequences, lineage trajectories, genetic regulatory networks, cell line pseudo-timelines, and temporal sequences of gene expression. In an embodiment, methods and systems of the invention continue to identify genes and corresponding guide RNA sequences involved in cell fate specification of a cell type, or an enhanced phenotype of a cell type. In a preferred embodiment, the genes are the minimal genes and the guide RNAs are the minimum effective set. In another embodiment, a collection of genes (i.e., a gene module) associated with affecting a particular phenotype may be identified. In an embodiment, the gene module may also include the temporal sequence of expression of the genes of the module. The module may be utilized to obtain the phenotype in any cell type.

Multiple approaches may be employed to identify one or more genes involved in directing cell differentiation, thereby engineering cell fate. For example, machine learning may be used to predict genes or genetic regulatory networks whose alteration activates, represses, or modifies transcriptional networks to produce target cell types. When machine learning is applied, training data may include data from the database or any other source of data representing various stages of the natural development of the starting cells to the mature cell type of a cell line. Training the machine learning algorithm may include providing data from a plurality of sources (a training data set) to the machine learning algorithm and optimizing parameters of the machine learning algorithm until the machine learning algorithm produces output describing the minimal genes, the temporal sequence, and the sequences of the minimum guide RNAs to achieve a cell type.

As such, applications and methods of this disclosure may also include a computer-implemented method, e.g., utilizes a computer system that includes a processor and a computer-readable storage medium. The processor of the computer system executes instructions obtained from the computer readable storage device to perform the analysis receiving data from a plurality of sources to identify, for example, the minimal gene targets to differentiate a cell to a desired cell type. For example, applications of the present disclosure relate to advanced analytics (such as machine-learning) tools, systems and methods for processing data from a database, or a multitude of databases, and provides an adaptive learning processor. The disclosed processor is configured to update and optimize its logic in response to receiving electronic data from multiple sources, for example, genetic databases, user input, and experimental data related to the effectiveness of the identified gRNA on targeting the gene for optimal gene expression, or the effectiveness of the identified genes to differentiate a cell.

Advantageously, embodiments of the present disclosure provide a self-learning processor that is capable of performing adaptive learning to optimize future prediction of, for example, the effectiveness of gene targets and of different gRNA sequences. Accordingly, the disclosed system provides increasingly accurate and valuable results that allow for optimized gene targets, optimized gRNAs, and optimized temporal gene expression sequences to differentiate a cell and ultimately direct cell fate specification. Cell fate specification can be that of any cell type (or subtype) within a cell line.

Certain embodiments include a combinatorial approach in which CRISPRa/i regulates expression of some factors in combination with the direct introduction or otherwise induced expression of other factors. Methods may include initiating expression of, or introducing, additional gene products identified by methods of the present invention as being necessary to promote differentiation of the one of the plurality of stem cells into the viable cell or progeny thereof. Expression of at least one of the additional gene products may be initiated by introducing a corresponding gene using, e.g., a PiggyBac transposon; introducing a corresponding gene via a plasmid or viral vector; or introducing an mRNA encoding the gene product. The additional gene products may be introduced as a protein to the one of the plurality of stem cells. The vector may include a viral vector, a plasmid, or transposable element. Optionally, the vector further has a selection marker, and the method includes selecting for cells transformed by the vector prior to an isolating step. The cells may be selected for transformation by the vector prior to introducing the one or more guide RNAs. In an illustrative embodiment, the gene product is a transcription factor and the transcription regulator under guidance of the dCas protein and the corresponding guide RNAs results in differentiation of the one of the plurality of stem cells into a neuron. In another embodiment, the one of the plurality of stems cells may be differentiated into a dopaminergic neuron.

The disclosed systems and methods allow for the identification and characterization of targets involved in cell differentiation. These methods may be used to identify targets that can be activated, inhibited, or altered to produce cells of any target phenotype from any starting cell type. Through the application of the disclosed methods, stem cells can be transformed into specific cell types that may serve as, or may produce, useful therapeutic agents for the treatment of various diseases. As such, stem cell treatments will benefit from the ability to intentionally and efficiently direct cell fate—the inducement of stem cells to differentiate into the desired target phenotype. Additionally, methods of the disclosure may be useful for the production of artificial cells with synthetic but desired phenotypes.

### BRIEF DESCRIPTION OF THE DRAWINGS

FIG. 1 diagrams steps of a screening method.

FIG. 2 shows an exemplary CRISPRa complex with an activator domain.

FIG. 3 shows an exemplary CRISPRi complex with an inhibitor domain.

FIG. 4 shows a CRISPR complex that recruits coactivator or corepressor proteins.

FIG. 5 diagrams directed cell fate specification of stem cells to a target phenotype.

FIG. 6 shows sequential differentiation of pluripotent stem cells into beta cells.

FIG. 7 depicts bar graph of an exemplary RT-qPCR of CRISPRa activation of target gene NEUROD1 in iPSCs using gRNA sequences predicted by methods of the invention.

FIG. 8 depicts bar graph of an exemplary RT-qPCR of CRISPRa activation of target gene NEUROG3 in iPSCs using gRNA sequences predicted by methods of the invention.

FIG. 9 presents a timeline of initial inducible neuron induction as an example.

FIG. 10 depicts a bar graph of exemplary RT-qPCR data of day three (3) cell differentiation of neurons.

FIG. 11 depicts exemplary immune-fluorescence images of day three (3) cell differentiation of neurons.

FIG. 12 depicts a bar graph of exemplary RT-qPCR data of day seven (7) cell differentiation of neurons.

FIG. 13 depicts exemplary immune-fluorescence images of day seven (7) cell differentiation of neurons.

FIG. 14 illustrates an exemplary t-SNE graph depicting a cell clustering analysis using single-cell RNA sequencing data from day ten (10) cell differentiation neurons.

FIG. 15 depicts a bar graph of the neuron GRN status of the nine (9) clusters of FIG. 14.

FIG. 16 depicts four (4) t-SNE plots mapping genes NEUROD1, NEUROG3, NANOG and POU5F1 and the nine clusters of cells to show the gene expression of those genes in those clusters of cells.

FIG. 17 depicts a graph of the classification of neuronal subtypes (x-axis) and the percentage of cells in each cluster (y-axis).

FIG. 18 illustrates an exemplary t-SNE graph depicting a cell clustering analysis using single-cell RNA sequencing data from the developing human midbrain.

FIG. 19 illustrates an exemplary t-SNE graph depicting cell subtype clusters using previously established gene signatures of neural subtypes.

FIG. 20 is a graphical representation of the gene enrichment (y-axis) of the identified subtypes (x-axis) in FIG. 19.

FIG. 21 illustrates an exemplary model depicting differentiation trajectories of the neural subtypes.

FIG. 22 depicts four (4) t-SNE plots mapping genes HMGA1, HMGB2, OTX2 and PBX1 and the different subtypes of cells to show the gene expression of those genes in those subtypes of cells.

FIG. 23 depicts three the top 13 up-regulated and top 13 down-regulated genes responsible for establishing the GRNs responsible for each cellular subtype identity as ranked by their respective GRN score.

FIG. 24 shows the exemplary mapping of the top level genes of the gene regulatory networks of the differentiation pathways from a neural progenitor cell to hDA1 and hDA2 subtypes.

FIG. 25 illustrates the predicted relative expression levels of the top level genes associated with mature dopaminergic neurons plotted across time (right), and the derivate of these expression levels (left) identifies inflection points in gene expression.

FIG. 26 provides the results of the CellNet analysis of the predicted manipulation of GRNs and resulting verification of cell line.

FIG. 27 provides the predicted gene regulation analysis of MYT1L and BASP3 during hDA2 differentiation.

FIG. 28 depicts a bar graph of the GRN status over time of NPCs and neurons during differentiation.

FIG. 29 illustrates a detailed block diagram of electrical systems of an example computing device in accordance with an example embodiment of the present disclosure.

FIG. 30A depicts immune-fluorescence images of the gene expression of intermediate neurons.

FIG. 30B depicts immune-fluorescence images of the gene expression of dopaminergic neurons.

FIG. 31 depicts immune-fluorescence images of the gene expression of day 35 dopaminergic neurons.

FIG. 32 depicts a bar graph of the amount of dopamine secretion of dopaminergic neurons of the present invention and a control.

### DETAILED DESCRIPTION

The invention provides screening methods for identifying targets involved in cell differentiation. Methods of the invention include introducing into stem cells, such as pluripotent stem cells, complexes that include guide RNAs and one or more effector domains to cause differentiation into a target phenotype. Cells demonstrating the desired target phenotype may then be selected and enriched for further analysis. One approach uses single-cell NGS of the gRNAs present in the single-cells demonstrating the target phenotype to produce sequence reads. Those sequence reads may then be mapped to particular loci of the genome to identify targets involved in cell differentiation. In another approach, sequences of the gRNAs may already be known, for example, if the gRNAs were designed from a database or purchased for use in the screening method. When those barcoded gRNAs are present in the cells with the target phenotype, the gRNA sequences may be mapped to various loci for target identification. In various embodiments, if one barcoded gRNA is present within a single cell, the transcriptomic or epigenomic effects of the activating (i.e., CRISPRa) or inhibiting (i.e., CRISPRi) complex targeted by that specific gRNA may be directly characterized. In addition, if multiple barcoded gRNAs targeting the same or different genes are present within a single cell, their collective genetic interactions can be used to identify networks of targets that are integral to directing cell fate and function.

The invention provides methods and systems for directing cell fate. Methods of the invention include identifying genes that are involved in directing cell fate specification of a desired cell type. Using methods of the invention, a minimal number genes determined to be responsible for directing cell differentiation of the desired cell type are selected as target genes and sequences of a minimum number of guide RNAs corresponding to each of the genes are identified. Methods of the invention include introducing into stem cells, such as pluripotent stem cells, complexes that include the guide RNAs and a Cas protein to cause differentiation into the desired cell type. The differentiated cells are characterized by methods to identify cell traits and their cell types identified by comparing known cell traits of cell types to that of the differentiated cells. If the desired cell type is not achieved in the first cycle of design-test-characterize, the cycle is repeated and each time a minimal number of the genes is identified as being responsible for directing cell differentiation of the desired cell type. The genes identified in the first cycle, may also be identified in subsequent cycles. Cycles of the method may be repeated to further enhance a phenotype of the cell type.

One approach to identify genes suspected to direct cell fate of a desired cell type is to perform a literature search. In another approach, inputs from various data sources are analyzed using bioinformatics analysis and genes are identified as directing cell fate of the desired cell type. A minimal number of the genes are selected and a guide RNA sequences to target each of the genes are then identified by analyzing the data. In one approach, identifying the minimum guide RNAs includes introducing into each of a plurality of cells a Cas protein and a guide RNA complex to produce a viable cell, or progeny thereof, measuring gene expression of the target in the viable cell to identify a minimum number of guide RNAs causing optimal gene expression of the target gene. In another approach the minimum set of guide RNAs are identified by bioinformatics analysis of the data. In another approach, sequences of the gRNAs may already be known, for example, if the gRNAs were designed from a database or purchased for use in a screening method.

Once the minimum set of guide RNAs are identified, they are complexed with a Cas protein and introduced to the stem cells to direct cell differentiation of the desired cell type.

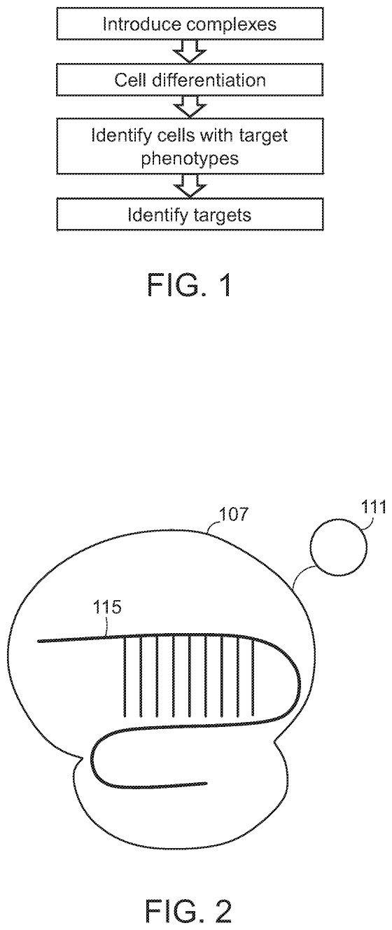

FIG. 1 diagrams steps of a screening method. In the method, complexes that include gRNAs and effector domains are introduced into starting cells, which are to be differentiated into a target phenotype. Once introduced into the stem cells, the complexes bind DNA and cause an activating or inhibiting activity and that activity may be used identify targets involved in cell differentiation. The complexes may include any Cas protein forming a complex with the gRNAs and effector domains, although in preferable embodiments, the Cas protein is a dCas.

A Cas9 that is catalytically active cleaves DNA via its HNH and RuvC nuclease domains. When the Cas9 nuclease has two functional domains and both of these domains are active, the Cas9 causes a double stranded break in the DNA. Thus, a Cas protein may be targeted to a specific location by forming a complex with a gRNA that includes a ˜20-bp guide sequence that is substantially complementary to a genetic locus. It is understood that gRNA includes gRNA with a trans-activating RNA (tracrRNA) as well as the use of a single guide RNA (sgRNA). In contrast, in dCas9, the HNH and RuvC nuclease domains are modified to disable their DNA cleaving activity, resulting in a dCas that retains its DNA binding ability but not its DNA cleaving activity. For example, point mutations may be introduced at catalytic residues (D10A and H840A) of the gene encoding Cas9. Complexes including dCas9, gRNA, and one or more effector domains can therefore take advantage of the DNA binding activity of the Cas9 protein and the DNA targeting ability of gRNA to intentionally bring the effector domains to target loci to cause cell differentiation into the target phenotype.

It is appreciated that any Cas protein that forms a complex with and is guided by the gRNA may be used, for example, Class II Cas proteins such as Cas9 and Cpf1. Cas proteins with single-subunit effectors are known as Class 2. These are then subdivided even further into type II (e.g., Cas9) and type V (e.g., Cpf1). Cas proteins include Cas9, Cpf1, C2c1, C2c3, and C2c2, and modified versions of Cas9, Cpf1, C2c1, C2c3, and C2c2, such as a nuclease with an amino acid sequence that is different, but at least about 85% similar to, an amino acid sequence of wild-type Cas9, Cpf1, C2c1, C2c3, or C2c2, or a Cas9, Cpf1, C2c1, C2c3, or C2c2 protein with a linked to an accessory element such as another polypeptide or protein domain (e.g., within a recombinant fusion protein or linked via an amino acid side-chain) or other molecule or agent.

C2c1 (Class 2, candidate 1) is a type V-B Cas endonuclease that has been found. Examples of C2c1 have been indicated to be functional in E. coli. tracrRNAs (short RNAs that help separate the CRISPR array into individual spacers, or crRNAs) were required. As is the case for Cas9, with C2c1, the tracrRNA may be fused to the crRNA to make a single short guide, or sgRNA. C2c1 targets DNA with a 5′ PAM sequence TTN.

C2c3 (Class 2, candidate 3) is a type V-C Cas endonuclease that clusters with C2c1 and Cpf1 within type V. C2c2 was found in metagenomic sequences, and the species is not known.

C2c2 (Class 2, candidate 2) is a type VI Cas endonuclease. C2c2 has been indicated to make mature crRNAs in E. coli. See Shmakov, 2015, Discovery and functional characterization of diverse class 2 CRISPR-Cas systems, Mol Cell 60(3):385-397, incorporated by reference.

In one embodiment, the complexes introduced include a dCas9 protein that forms a complex with the gRNAs and effector domains. For example, the effector domain may be an activator, an inhibitor, or a domain that recruits coactivator or corepressor proteins to the complex, for instance, by acting as a scaffold.

Examples of effector domains that acts as activators include the VP16 activation domain (VP16), VP48 (three copies of VP16), VP64 (four copies of VP16), VP96 (six copies of VP16), VP160 (ten copies of VP16), VP192 (twelve copies of VP16), the p65 activation domain (p65AD), VPH (VP192, p65, and heat shock factor 1 (HSF1)), VPPH (VP192, a catalytic core of human acetyltransferase p300 (p300), p65, and HSF1), and VPR64. VPR64 is a tripartite activator domain that includes VP64, p65AD, and the Epstein-Barr virus R transactivator (Rta). Examples of effector domains that acts as inhibitors include the Krüppel-associated box (KRAB), four concatenated mSin3 interaction domains collectively labelled (SID4X), and max-interacting protein 1 (MXI1).

An example of an effector domain that recruits subsequent effector domains is a SunTag. In a dCas9-SunTag complex, dCas9 may be fused with a SunTag array made of ten copies of a small peptide epitope. The SunTag array acts as a scaffold to recruit multiple copies of effector proteins. The effector proteins recruited may be, for example, VP64 activator proteins fused to a cognate single-chain variable fragment (scFV).

In another example, a synergistic activation mediator (SAM) effector domain is included in the complexes, in which two bacteriophage MS2 RNA aptamers (MS2s) are added to the tetraloop and second stem-loop of the gRNA complexed with dCas9. These MS2 RNA aptamers are able to recruit MS2 coat proteins (MCPs). MCPs are MS2 coat proteins fused to VP64, p65AD or HSF1 activators.

In various embodiments, selection marker domains may be included to assist in selecting and enriching for cells with stable uptake of the complexes. For example, the selection marker may be a fluorescent marker (GFP), or drug resistant marker (blasticidin). If such selection markers are used, cells may be selected for stable uptake of the complexes, for example, by fluorescence-activated cell sorting (FACS) if a GFP selection marker was employed or by drug screening if the blasticidin selection marker was employed.

In some embodiments, effector domains may be directly fused to dCas forming, for example, dCas9-VP16, dCas9-VP48, dCas9-VP64, dCas9-VP96, dCas9-VP160, dCas9-p65, dCas9-VPH, dCas9-VPPH, dCas9-VPR64, dCas9-KRAB, dCas9-SID4X, or dCas9-MXI1. In other embodiments, such as dCas9-SunTag and dCas9-SAM, the effector domains are not directly fused to dCas9, but instead recruit other proteins to cause an activating or inhibiting activity. It is understood that include effector domains may manipulate epigenetic modifications such as histone acetylation or methylation and DNA methylation. For example, inhibiting activity may be caused by dCas9-LSD1 (Lys-specific histone demethylase 1) and activating activity may be caused by dCas9-p300.

The complexes may be introduced into the stem cells in various ways and by any suitable method, for example, by transfection or transduction. The complexes may be introduced into the cells as an active protein—or ribonucleoprotein (RNP) in the case of a Cas-type nuclease—or encoded in a vector, such as a plasmid or mRNA, in a viral vector, such as adeno-associated virus (AAV), or in a lipid or solid nanoparticle. The complexes may be transfected into cells by various methods, including viral vectors and non-viral vectors. Viral vectors may include retroviruses, lentiviruses, adenoviruses, and adeno-associated viruses. It should be appreciated that any viral vector may be incorporated into the present invention to effectuate delivery of the complex into a cell. Use of viral vectors as delivery vectors are known in the art. See for example U.S. Pub. 2009/0017543, incorporated by reference. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355, each incorporated by reference) and lipofection reagents are sold commercially (e.g., Transfectam and Lipofectin).

In one embodiment, dCas, effector domains, and gRNAs are transcribed in vitro, complexed to form an RNP complex, and introduced into the stem cells by any suitable method, for instance, electroporation or cationic lipid transfection. dCas and effector domains may also be transcribed in vitro and introduced into the stem cells separate from transcribed gRNAs. In another embodiment, mRNA encoding dCas may be co-introduced with the gRNAs. The dCas encoded by the mRNA may include one or more effector domains. Also, the dCas may be constitutively expressed. The mRNA encoding dCas may also encode gRNA. In another embodiment, dCas and gRNAs are introduced into stem cells by transduction with or separate from the gRNAs. For example, dCas and gRNA may be attached to a single lentiviral backbone and introduced by lentiviral transduction.

Methods of the disclosure identify guide RNAs that differentiate stem cells to a target cell type. In very high throughput embodiments, guide RNAs can be screened in a shotgun approach in which very large numbers of effectively-random guide RNAs are screened to discover those that are useful for differentiation. In preferred embodiments, large numbers of candidate guide RNAs that may be identified using some a priori knowledge or methodological searching are used and screened. Methods may include selecting an abundance of guide RNAs that target promotor areas of genes suspected to be involved in differentiating cells to a specified differentiated cell type, and then using the methods to identify, from among the abundance (e.g., hundreds or thousands) of the guide RNAs and effective set (e.g., 1 to 40 per gene for 1 to about 5 different genes associated with the specified differentiated cell type) of guide RNAs, in which an effective set is a set of one to a few dozen guide RNAs that can be delivered with a CRISPRa/i protein to effectively differentiate stem cells into the specified differentiated cell type.

Methods may include predicting the sequence of guide RNAs that target promotor areas of genes identified as involved in differentiating cells to a specified differentiated cell type, and then using the methods to identify the minimum effective set (e.g., 1 to 5 per gene for 1 to about 5 different genes associated with the specified differentiated cell type) of guide RNAs, in which the effective set can be delivered with a CRISPRa/i protein to effectively differentiate stem cells into the specified differentiated cell type.

Selecting that initial abundance of guide RNAs can include a process that includes (1) first a literature search to identify genes suspected to be involved in differentiating cells to the specified differentiated cell type followed by (2) a genomic database search (e.g., in GenBank or Ensembl) to identify suitable guide RNA targets (e.g., unique or nearly-unique 20 base stretches, adjacent to a protospacer adjacent motif, within putative promoter regions of genes identified in step 1. Searching the genomic database may be performed by computer software such as a Perl script or Python code that applies the rules for Cas endonuclease guide RNA targeting and identification of promoter regions for coding strands to identify putative targets. That same code may perform step 1 (so-called literature search) by searching keywords in GenBank annotations to identify coding regions that have been labelled with keywords specific to a desired trait or cell type. The set of guide RNAs (which may number in the thousands or hundreds of thousands) identified by the in silico selection methodology (steps 1 and/or 2) may then be provided as RNA molecules for delivery to the stem cells. The desired RNAs may be ordered e.g., from a service such as Integrated DNA Technologies, Inc. (Skokie, Illinois) or synthesizing the RNAs on a synthesis instrument. The guide RNAs are introduced into the stem cells with the dCas protein linked to a transcription regulator (e.g., an effector domain).

The process may also include (3) analysis of the data to identify a temporal sequence of expression to direct cell fate specification of a subtype of the desired cell type. In yet another embodiment, the analysis for step 3 may also include implementation of software/algorithms to reconstruct and verify the subtype is achieved. If the subtype is not achieved, steps 1-3 are repeated until the subtype is achieved.

The dCas-linked effector domain protein and the guide RNA may be delivered into stem cells in any suitable format and using any suitable delivery technology. For example, the protein may be introduced as a DNA vector (e.g., plasmid or viral vector), in the mRNA sense, or as a formed protein. The guide RNA may be introduced in the same or different DNA vector, as a free guide RNA, or complexed with the protein in the form of RNP. Whichever format is used, the molecular structures to be delivered may further be complexed with, linked to, or encapsulated by any suitable delivery reagent such as one or more nanoparticles (such as a solid lipid nanoparticle, a micelle, metal particles, polymer particles, or a liposome), PEG, or biological macromolecules such as sugars or intra-cellular trafficking proteins such as nuclear localization signals. The molecule structures to be delivered may further be delivered using any suitable technology. In preferred embodiments, screening methods of the disclosure scale up to high throughput, and allow multiple replicates to be performed in parallel (e.g., tens or dozens or greater of 384-well plates are filled with experimental aliquots using, e.g., liquid handling robots). Delivery of the molecular structures to such quantities of stem cells may be best served using a technology that scales up well to high-throughput applications such as laser excitation of plasmogenic substrates. In such technologies, a reactive substrate is presented in proximity to stem cells and the payload to be delivered (e.g., dCas-effector domain RNP, or a nucleic acid encoding the dCas-effector domain RNP, and guide RNAs). The substrate is excited with a laser. Preferably, the substrate includes physical structures such as tetrahedral peaks (e.g., a grid comprising thousands of peaks over an area of plasmogenic material on the order of 10 mm×10 mm. Laser excitation of the surface induces temporary poration of the stem cells, allowing the payload to diffuse into the stem cells. Such a technology can provide the throughput necessary to introduce a dCas protein linked to a transcription regulator, or nucleic acid encoding the same, and a guide RNA, into each of thousands or tens of thousands or more stem cells, allowing for similar quantities of guide RNAs to be synthesized (e.g., using a benchtop RNA oligo synthesis instrument) and delivered to the stem cells. This technology for efficiently delivering functional cargo to millions of cells within minutes may be offered under the trademark NANOLAZE. See Saklayaen, 2017, Intracellular delivery using nanosecond-laser excitation of large-area plasmonic substrates, ACS Nano 11:3671-3680, incorporated by reference.

In certain embodiments, once the payload is delivered into the stem cells, methods of the disclosure include differentiating one or more of those stem cells into cells with a desired phenotype.

Next, cells with the desired target phenotype may be identified and isolated, such as by FACS or drug screening, depending on the selection markers used. In various embodiments, the cells may be sorted as whole populations for analysis or as single cells for isogenic expansion or single-cell analysis. For example, in single-cell analysis, a single cell with the target phenotype is lysed, its genomic DNA is isolated, whole-genome-amplification is performed, a sequencing library is constructed, and the DNA of that single cell is sequenced.

In cell selection, either or both of a ‘biased’ and an ‘unbiased’ approach may be applied for selection and analysis. The ‘biased’ approach involves selecting for cells that are viable and that demonstrate the target phenotype. The ‘unbiased’ approach involves only selecting for cells that are viable. Subsequent analysis may differ depending on the approach selected.

In certain embodiments, sequencing is by single-cell NGS. Single-cell NGS generally refers to non-Sanger-based high throughput DNA sequencing technologies applied to the genome of a single cell, in which many (i.e., thousands, millions, or billions) of DNA strands can be sequenced in parallel. Examples of such NGS sequencing includes platforms produced by Illumina (e.g., HiSeq, MiSeq, NextSeq, MiniSeq, and iSeq 100), Pacific Biosciences (e.g., Sequel and RSII), and Ion Torrent by ThermoFisher (e.g., Ion S5, Ion Proton, Ion PGM, and Ion Chef systems). It is understood that any suitable next-generation DNA sequencing platform may be used for single-cell NGS as described herein.

Multiple approaches may be employed to identify one or more targets involved in directing cell differentiation. In one example, after single-cell NGS is performed on the gRNAs following a ‘biased’ approach to select cells demonstrating the target phenotype, the NGS sequence data is de-multiplexed using unique index reads and barcoded gRNA counts may be determined by only perfect-match sequencing reads. These gRNAs may then be mapped to loci of a genome, to identify candidate loci targets which are involved in cell differentiation. Whether a ‘biased’ or an ‘unbiased’ approach is applied to cell selection, machine learning may be applied to the NGS sequence data produced to identify and predict combinations of genetic loci targeted by the complexes. For example, machine learning may be used to predict networks of interrelated genes whose alteration activates, represses, or modifies transcriptional networks to produce the target phenotypes. When machine learning is applied, training data for the machine learning may include data from either or both of the ‘biased’ and ‘unbiased’ approaches, as well as other publicly-available sequencing data from various stages of the natural development of the starting cells of the target phenotype. In an example, features of the target phenotype may then be split into individual parameters to categorize gRNAs identified or predicted to be involved in causing those phenotypic features in the stem cells.

In one embodiment, where each cell received one gRNA that was barcoded, NGS sequencing and subsequent analysis may be used to directly characterize the transcriptomic or epigenomic effects of the dCas9-effector domain targeting with that specific gRNA. For example, if a dCas9 fused to a VPR activator domain is directed to a particular loci and activates a gene which causes an iPS cell to differentiate into a beta cell, an NGS sequence read of that specific gRNA may be mapped to a loci to identify a target whose activation is involved in directed cell fate specification to the target phenotype.

In another embodiment, where each cell received multiple gRNAs that were barcoded, each targeting the same or different genes present within a single cell, NGS sequencing and analysis may be used to determine whether and how their collective interactions form a network involved in directed cell fate specification to the target phenotype.

In another aspect of the invention, a method is provided for identifying targets involved in cell differentiation. The method includes introducing complexes that include gRNAs and one or more effector domains into stem cells, identifying at least one of the gRNAs or effector domains that caused at least one of the stem cells to differentiate into a target phenotype, and correlating nucleic acid sequences of the gRNAs to loci of a genome, thereby identifying one or more targets involved in causing cell differentiation into the target phenotype. Introducing the complexes into stem cells and identifying at least one of the guide RNAs or effector domains that caused at least one of the stem cells to differentiate into the target phenotype may be performed by any of the methods discussed. In this method, correlating a nucleic acid sequence of the guide RNAs to loci of a genome may involve performing any of the single-cell NGS methods described and relating nucleic acid sequences of the gRNAs to target loci. Alternatively, if sequences of the gRNAs are known, such as if the gRNAs were commercially obtained or designed such that the nucleic acid sequences of each are known, then correlating those sequences to loci of a genome may be performed without NGS. In any event, one or more targets involved in causing cell differentiation into the target phenotype may be identified by correlating nucleic acid sequences of the gRNAs to loci of a genome, where the gRNAs are present in selected cells with the target phenotype.

In certain embodiments, once the payload is delivered into the stem cells, methods of the disclosure include differentiating one or more of those stem cells into cells of a desired cell type. In other embodiments, the stem cells are further differentiated into cells of a desired phenotype.

Next, differentiated cells having the desired cell type may be identified. Cell types are identified by specific cell traits that have been previously identified as characteristic of a certain cell type. Cell traits may include cell morphology, chromosome analysis, DNA analysis, protein expression, RNA expression, enzyme activity, cell-surface markers, or a combination thereof. Each of the differentiated cells produced by methods of the invention may be characterized by cell traits. Characterizing the cells may include identifying cell traits by staining the cells with a marker for the desired characteristic, and sorting the cells using, for example, a fluorescence-activated cell sorting instrument, a magnetic bead-based purification, others, or a combination thereof. In another embodiment, characterizing the cells may include identifying cell traits by measuring gene expression in the cell or progeny thereof. Gene expression includes one or more of: quantifying expression levels via RNA-Sequencing; measuring gene expression via single-cell RNA sequencing; or evaluating DNA-protein interaction via chromatin immunoprecipitation and DNA sequencing (ChIP-seq). The methods may include determining fold-change in expression level of a transcript associated with a marker by normalizing read counts from the measuring against control read counts. The methods may also include comparing transcriptomes of individual cells to assess transcriptional similarities and differences between the cells.

The cell type of each of the differentiated cells may be determined by comparing the identified traits of each of the cells to the known traits of a cell type. The methods may also include identifying cell type by comparing transcriptomes of the cells to assess transcriptional similarities and differences between the cells and may include clustering like cells. In an embodiment, the desired trait includes a specified differentiated cell type and the marker includes a protein expressed by the differentiated cell type. In an example, the desired trait may be a neuronal phenotype, and marker one or more of the presence of beta III tubulin and DAPI and the absence of Oct4. In another example, the desired trait may include an inducible neuron phenotype, and the marker the presence of beta III tubulin.

Optionally, the cell type of the differentiated cells can be identified by other methods such as FACS or drug screening, depending on the selection markers used. In various embodiments, the cells may be sorted as whole populations for analysis or as single cells for isogenic expansion or single-cell analysis. For example, in single-cell analysis, a single cell with the target phenotype is lysed, its genomic DNA is isolated, whole-genome-amplification is performed, a sequencing library is constructed, and the DNA of that single cell is sequenced.

In certain embodiments, sequencing is by single-cell NGS. Single-cell NGS generally refers to non-Sanger-based high throughput DNA sequencing technologies applied to the genome of a single cell, in which many (i.e., thousands, millions, or billions) of DNA strands can be sequenced in parallel. Examples of such NGS sequencing includes platforms produced by Illumina (e.g., HiSeq, MiSeq, NextSeq, MiniSeq, and iSeq 100), Pacific Biosciences (e.g., Sequel and RSII), and Ion Torrent by ThermoFisher (e.g., Ion S5, Ion Proton, Ion PGM, and Ion Chef systems). It is understood that any suitable next-generation DNA sequencing platform may be used for single-cell NGS as described herein.

Machine learning may be applied to the data obtained from the characterization steps of any of the methodologies used to characterize the cell identity to predict combinations of genes to be targeted by the complexes. For example, machine learning may be used to predict networks of interrelated genes whose alteration activates, represses, or modifies transcriptional networks to produce the desired cell types. When machine learning is applied, training data for the machine learning may include data obtained from systems of algorithms, publications, public data sets (e.g., gene expression data sets), cell type profiles, scRNA-seq (single-cell RNA sequencing) expression data, results of internal analysis, and any other data relevant data sources.

One way of making use of the disclosed methods of the invention may be to optionally utilize the output of a trajectory inference system of algorithms, CellRouter (Lummertz da Rocha, 2018) or that of DPT (diffusion pseudotime) in Nature Methods, or that of Monocle, published in Nature Biotechnology in 2014 and Nature Methods 2018 in August 2017 to identify additional target genes to differentiate stem cells to the desired cell type. The outputs can be added to the data for more refined analysis.

In an example, a cluster analysis of the invention clusters single-cells such that each cluster shows differential gene expression signatures. Genes preferentially expressed in each cluster, including known neuronal genes, have shared/similar features such as gene expression, phenotype, and genetic pathways. From the cluster analysis, one can identify networks of genes that exhibit features with a high degree of similarity (relatedness). Based on the high degree of similarity, cell type lineage trajectories and the associated genes can be identified. By way of example, graph theory algorithms can be utilized, and one way of making use of the methods of the invention is to utilize the outputs of those described in CellRouter, to identify cell network similarities. Cell types of differentiated cells are identified by identifying transcriptome similarities amongst the cells, where the cell clusters are representative of different cell types of the lineage. Community-detection algorithms (e.g., the Louvain method) may be used to identify inter-connected cells, and therefore define cell types. As such, methods of the invention utilize graph theory algorithms to cluster cell types. Clusters of the cell types can be depicted visually in the graph, such as t-SNE plot. Using previously identified cell type gene signatures, the cell types can be further categorized, for example.

Another way of making use of the disclosed methods of the invention is to utilize flow network algorithms, such as those described by CellRouter to then identify cell type trajectories. The structure of the network is a directed graph and the vertices are called nodes and the edges are called arcs and represent connections between the nodes. G=(V, E), where V is a set of vertices and E is a set of V's edges—a subset of V×V—together with a non-negative function c: V×V→ [symbol] ∞, is the capacity function. If two nodes in G are distinguished, a source s and a sink t, then (G, c, s, t) is called a flow network. A flow must satisfy the restriction that the amount of flow into a node equals the amount of flow out of it, unless it is a source, which has only outgoing flow, or sink, which has only incoming flow. Transformations known in the art can be used to optimize the network. Here, each node represents a single-cell and each edge connects cells that are phenotypically similar. Phenotypic similarities are quantified by the edge's weights. As such, the entire network, or graph will provide cell-to-cell similarities, thereby identifying paths connecting cell types (the cell clusters) and therefore defining differentiation trajectories.

Using a gene expression analysis of the data sets of the cell clusters, significant overlaps, or commonalities, in the data, for example, overlaps in genetic content are identified. Gene expression amongst different cell types, or cell clusters, can be compared to identify overlap of genes amongst the cell types. In certain aspects of the invention, the gene modules identify an overall functional congruity between cell clusters allowing for the identification gene expression patterns. In a preferred embodiment, differentiation pathways between the cell types are identified. The genes are typically mammalian genes. The mammalian genes may correspond to mouse genes, human genes, or a combination thereof. Feature data (such as gene expression, phenotype, gene pathway, etc.) and genes may be used to form a matrix that will be used to exhibit the trajectory inference analysis. For example, the feature data is pre-processed to express each domain as a row and each feature as a column (or vice versa). For domains with continuous values such as gene expression, the features are the individual cells of which gene expression was measured, and each value in the matrix (Xij) represents the expression of gene i in a cell j. For domains with categorical values such as phenotypes, the features are the individual phenotypes, and each value in the matrix (Xij) is a binary indicator representing whether gene i is associated with phenotype j. All of the domain specific matrices are then combined column-wise. A distance metric is then applied to each pair of rows and each pair of columns in the matrix. In certain embodiments, the distance metric is ‘Distance=1-correlation’. However, it is understood that other standard distance metrics could be used (e.g. Euclidean).

Generally speaking, after a graph-based clustering analysis is applied to the gene expression data, the gene “clusters” can be displayed against certain feature categories (e.g. phenotype/gene expression ‘category’), which are then clustered to reflect commonality. In other embodiments, the gene modules (or clusters) are displayed against the cell different cell type trajectories. For example, phenotypes of immature dopaminergic neurons (hDAO) are grouped together in one cluster, and phenotypes of mature dopaminergic neurons (hDA1 and hDA2) patterning, morphology and growth are grouped in a separate cluster, etc. The degree of relatedness or commonality between the clustered cells and the cluster-specific genes (as determined by the cluster analysis) can then be highlighted on the resulting cluster matrix. For example, red may be used to indicate that the gene is associated with morphology and/or is expressed at high levels in the associated cell type indicated on the opposite axis; whereas blue may be used to indicate that the gene is associated with morphology, but and/or is expressed at low levels in a different cell type.

Methods of the invention assess several features (or parameters) of genes in order to determine their relationship to a cell type differentiation trajectory. The method includes ordering cell types from early to late stage differentiation. In certain embodiments, the features include gene expression, phenotypes, gene pathways, and a combination thereof. In a preferred embodiment, the trajectory is a developmental trajectory of a cell from an immature cell type to a mature cell type. As such, the cell types are ordered along a pseudo-timeline of cell development. In another embodiment, the trajectory is an intermediate trajectory from one cell type to another cell type. In another embodiment, the ranked genes are the genes necessary to direct cell differentiation. By clustering cells and identifying cluster-specific genes into feature specific groups and color-coding genes with high degree of relatedness, the resulting cluster matrix of the invention advantageously allows for visualization of groups of genes (the modules) that are strongly associated with phenotypes relating to particular cells (i.e. clusters of interest). Thus, cluster matrices of the invention allow one to quickly identify a detailed mapping of cellular differentiation pathways based on cell types' shown association (cluster) with one another. This mapping further allows for identification of genes responsible for establishing genetic regulatory networks (GRN) for a cell subtype, by further mapping the gene along the trajectories.

Methods of the invention include inputting the results of scoring the genes of the GRN into the systems described herein to predict transcriptional regulators. For example, a GRN score maybe assigned to each gene by implementing the CellRouter algorithm. The genes are assigned a GRN score based on their correlation with their progression of the identified trajectory, their correlation of their predicted gene targets, and the extent to which target genes are regulated during a particular trajectory. The up-regulated genes with the highest score and down-regulated genes with the highest score are selected and mapped to the genes of downstream GRNs to identify genes responsible for the cell fate. In an embodiment of the invention, at least 1 of the top ranked up-regulated genes and at least 1 of the down-regulated genes are identified. In a preferred embodiment, 10 to 20 up-regulated genes with the highest score and 10 to 20 down-regulated genes with the highest score are selected. The method also includes plotting the expression levels of the genes across the pseudo-timeline to identify inflection points in gene expression along the trajectory. As such, the method identifies genes and the temporal sequence of the gene expression to direct cell fate. In another embodiment, the genes identified are temporally expressed to direct the cell fate. The temporal expression of the genes regulates the expression of target genes associated with a specific cell type.

As a result of integrating both the GRN scoring and the psedo-timeline (or temporal sequence) into the methods described herein, a minimum number of genes and a temporal sequence of expression of the genes to direct maturation of a cell are identified.

To determine if the identified genes are capable of differentiating the stem cell into the desired cell type, methods of the invention analyze data obtained from a plurality of sources, including, for example, the output of CellRouter, to identify sequences of a corresponding minimum number of guide RNAs and induce stem cells with the guide RNAs complexed to Cas protein and may be repeated until the subtype is verified by the method. In another embodiment, the methods of the invention may be performed in silico, and may be repeated until the subtype is verified by the method.

The data obtained from the methods are inputted into the database and machine learning may also be applied to the data produced by analysis performed on the system or data sets inputted into the system to identify limited targets and corresponding minimal number of gRNAs to produce a desired cell type. For example, machine learning may be used to predict networks of interrelated genes whose alteration activates, represses, or modifies transcriptional networks to produce the target phenotypes using the database. When machine learning is applied, training data for the machine learning may include data from gene expression analysis, as well as other publicly-available sequencing data from various stages of the natural development of the starting cells of the target phenotype. In an example, features of the target phenotype may then be split into individual parameters to categorize gRNAs identified or predicted to be involved in causing those phenotypic features in the stem cells.

In another example, machine learning may be used to identify specific temporal sequence of activating and/or inhibiting target genes to differentiate cells. For example, the temporal sequence may include the introduction of a first set of one or more guide RNAs during a first period comprising one or more hours or days followed by introduction a second set of one or more guide RNAs during a second period comprising one or more hours or days. Optionally, the first set of one or more guide RNAs and the second set of one or more guide RNAs comprise wholly different guide RNAs and/or the first period and the second period do or do not overlap in time. In some embodiments, CRISPRa/i is used against a first set of targets during the first period, the first period comprising at least two days, and using CRISPRa/i against a second set of targets during the second period to differentiate the one of the plurality of stem cells into a dopaminergic neuron. As such the invention includes methods of introducing RNAs (e.g., with the dCas protein linked to the regulator) into at least one of the plurality of stem cells in a temporal sequence.

Upon identifying genetic targets and the temporal sequence, the machine learning system provides a report with a program for using the targets and the temporal sequence to allow for specified cell fate engineering. The program is for the sequential delivery of CRISPRa/i RNPs or transcription factors to the starting cell type (e.g. human pluripotent stem cells), along with sequence of the identified targets encoded in vectors with conditional modules that recapitulate the program necessary to derive the desired cell type/subtype. The report can also identify a gene module to be used on any type of cell to effectuate a specific phenotype.

Vectors can include integratable viral (e.g. lentivirus) non-viral (e.g. PiggyBac) methods, or non-integratable viral (e.g. Sendai virus) and non-viral (e.g. episomal) methods. In one embodiment, hiPSCs receive an episomal vector containing transcriptional regulators under different inducible promoters where the relative timing of expression of each factor or set of factors is achieved through exposure of the cells to different inducers in varying combinations across time to achieve cell fate specification. In another embodiment, an episomal vector contains a constitutively expressed CRISPRa/i with a guide RNA array where components of the array are inducible in varying combinations across time to achieve cell fate specification of the subtype.

In certain embodiments the invention allows for mapping of cell differentiation pathways or trajectories, for example from one cell type to a specific phenotype or subtype of the cell type and the cell subtypes are ordered from early to late stage development by clustering the cells by subtype. The ordering of the cell subtypes identifies a pseudo-timeline of cell development for the cell type. Those cell differentiation pathways allow the system to identify genes (or transcriptional factors) responsible for establishing the GRN. The genes are assigned a GRN score according and the genes (both up-regulated and down-regulated) with the top scores are mapped to downstream GRNs. Such mapping identifies the minimum number of to effectuate cell differentiation of a particular cell subtype. By further plotting the expression levels of the minimum number of genes across the pseudo-timeline, the system identifies the temporal expression sequence of the genes. Methods and systems of the presently disclosed invention allow for cell fate engineering by identifying a minimum set of target genes and their minimal effectors capable of specifying cell fate between any given cell type.

FIG. 2 shows an exemplary CRISPRa complex containing a dCas9 protein 107, gRNA 115, and effector domain 111, which is a transcription factor with an activating activity. As illustrated, activator domain 111 is fused to dCas9 107, which is complexed with gRNA 115, which will target the CRISPRa complex to a sequence-specific genomic location. dCas9 107 binds the DNA and allows effector domain 111 to cause an activating activity. It is understood that any effector domain that causes an activating activity may be employed. For example, activator domain 111 may be a VP16, VP48, VP64, VP96, VP160, p65AD, or VPR. Many CRISPRa complexes, each with many different gRNAs may be employed to target various and overlapping genomic sequences to achieve robust activation of endogenous target genes.

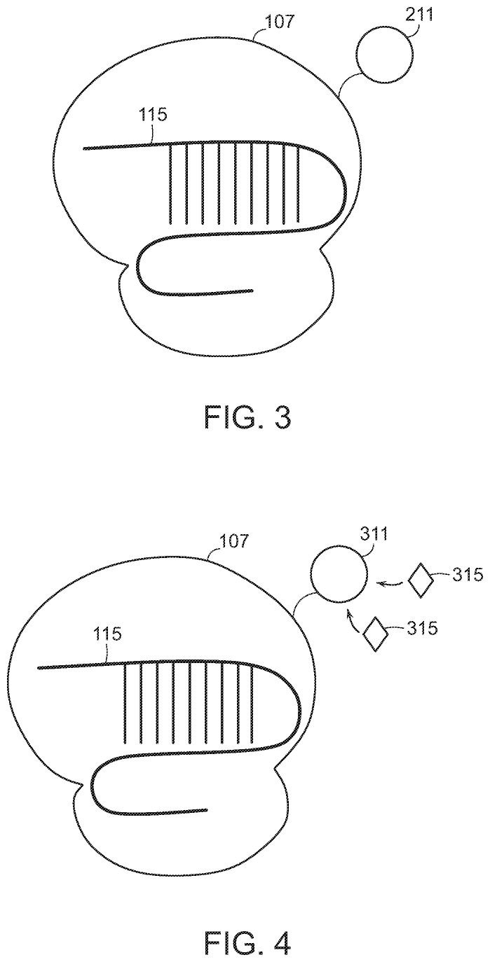

FIG. 3 shows an exemplary CRISPRi complex containing a dCas9 protein 107, gRNA 115, and effector domain 211, which is a transcription factor with an inhibiting activity. As illustrated, inhibitor domain 211 is fused to dCas9 107, which is complexed with gRNA 115, which will target the CRISPRi complex to a sequence-specific genomic location. dCas9 107 binds the DNA and allows effector domain 211 to cause an inhibiting activity. It is understood that any effector domain that causes an inhibiting activity may be employed. For example, inhibitor domain 211 may be a KRAB, a SID4x, or MXI1 protein. Many CRISPRi complexes, each with many different gRNAs may be employed to target various and overlapping genomic sequences to achieve robust repression of endogenous target genes.

FIG. 4 shows an exemplary complex including a dCas9 protein 107, gRNA 115, and effector domain 311 that recruits coactivator or corepressor proteins 315 to the complex. By recruiting multiple coactivator or corepressor proteins to the complex, more robust activation or inhibition of endogenous target genes may be achieved in comparison to complexes containing effector domains directly fused to dCas.

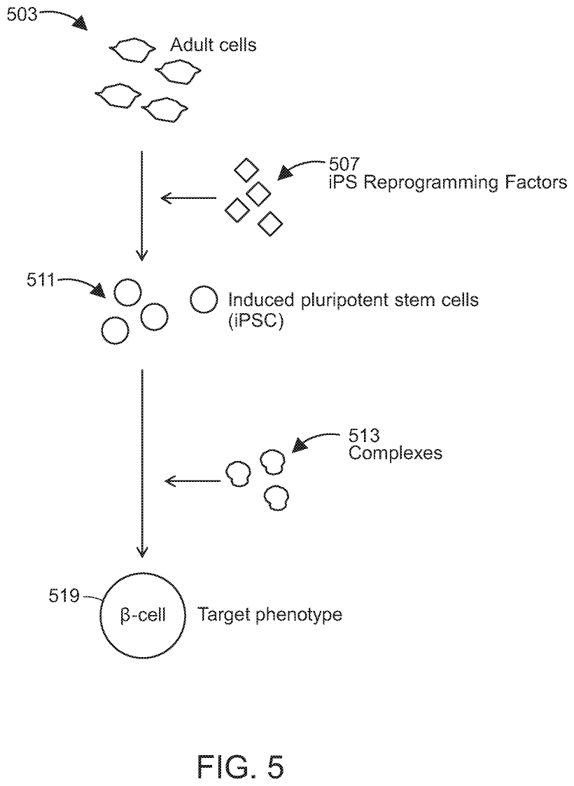

FIG. 5 shows a diagram of directed cell fate specification of iPS cells to cells of a target phenotype. As illustrated, adult cells 503 of any cell type may be modified by certain induced pluripotent stem cell reprogramming factors 507 to reprogram those adult cells to become iPS cells 511 capable of differentiating into any subsequent cell type. These iPS cells should be stable for the complexes introduced, for instance a CRISPRa complex for activating genes involved in cell differentiation or a CRISPRi complex for inhibiting genes involved in cell differentiation. As illustrated, the target phenotype is a beta islet cell 519, which is an insulin-producing cell. In this example, complexes 513 introduced into iPS cells 511 include dCas9 proteins fused to a -VPR tripartite activator domain. These dCas9-VPR ribonucleoproteins formed complexes with gRNAs targeting loci involved in directing the iPS cells to differentiate, producing synthetic beta islet cells 519.

Starting Cell Types

Methods of the invention may be applied to, but are not limited to the following example cell types: Human BC-1 Cells, Human BJAB Cells, Human IM-9 Cells, Human Jiyoye Cells, Human K-562 Cells, Human LCL Cells, Mouse MPC-11 Cells, Human Raji Cells, Human Ramos Cells, Mouse Ramos Cells, Human RPMI8226 Cells, Human RS4-11 Cells, Human SKW6.4 Cells, Human, Dendritic Cells, Mouse P815 Cells, Mouse RBL-2H3 Cells, Human HL-60 Cells, Human NAMALWA Cells, Human Macrophage Cells, Mouse RAW 264.7 Cells, Human KG-1 Cells, Mouse M1 Cells, Human PBMC Cells, Mouse BW5147 (T200-A)5.2 Cells, Human CCRF-CEM Cells, Mouse EL4 Cells, Human Jurkat Cells, Human SCID.adh Cells, Human U-937 Cells, Human HOS Cells Human Saos-2 Cells, Human U-2 OS Cells, Human MH7A Cells, Mouse 3T3-L1 Cells, Human BJ Cells Monkey COS-7 Cells, Human Neonatal Dermal Fibroblast Cells, Horse Embryonic Dermal Fibroblast Cells (NBL-6), Mouse Embryonic Fibroblast Cells (MEF), Human HT-1080 Cells, Human, IMR-90 Cells, Mouse L-929 Cells, Mouse NIH-3T3 Cells, Mouse PA317 Cells, Monkey, Vero Cells, Human WI-38 Cells, Mouse b-END.3 Cells, Human Endothelial Cells, Human HUVEC Cells, Rat PC-12 Cells, Human 253J Cells, Human J82 Cells, Human RT4 Cells, Human T24 Cells, Mouse F9 Cells, Mouse P19 Cells, Human ARPE-19 Cells, Human COLO 201 Cells, Human HCT 116 Cells, Human HCT15 Cells, Human HT-29 Cells, Human RKO Cells, Human SW480 Cells, Human WiDr Cells, Human 293A Cells, Hamster BHK-21 Cells, Human HEK 293 Cells, Canine, MDCK Cells, Rat NRK Cells, Human ChangX-31 Cells, Rat H-4-II-E Cells, Human Hep G2 Cells, Human Hep3B Cells, Human SK-HEP-1 Cells, Human SNU-387 Cells, Human BT-20 Cells, Human, HCC1937 Cells, Human Hs-578T Cells, Human Mammary Epithelial Cells, Human MCF7 Cells, Human MCF-ADR Cells, Human MDA-MB-231 Cells, Human SK-BR-3 Cells, Human T-47D Cells, Chinese Hamster CHO DG44 Cells, Chinese Hamster CHO-K1 Cells, Human SK-OV-3 Cells, Human, BxPC-3 Cells, Human PANC-1 Cells, Rat GH3 Cells, Human DU 145 Cells, Human LNCaP Cells, Human TSU-Pr1 Cells, Human PC-3 Cells, Human A549 Cells, Human BEAS-2B Cells, Human NCI-H23 Cells, Human NCI-H69 Cells, Human Calu-3 Cells, Human G-361 Cells, Human HN3 Cells, Human MEWO Cells, Human ARO Cells, Human FRO Cells, Human NPA Cells, Human A-431 Cells, Human HeLa Cells (ATCC), Human C-33 A Cells, Rat Cardiomyocyte Cells, Mouse, C2C12 Cells, Rat L6 Cells, Human Aortic Smooth Muscle Cells, Rat Astrocyte Cells, Rat Cortical, Astrocyte Cells , Rat C6 Glial Cells, Mouse Glial Cells, Rat Glial Precursor Cells , Human T98G Cells, Human U-87 MG Cells, Human SH-SYSY Cells, Human SK-N-MC Cells, Rat Primary Cortical, Neuron Cells, Mouse GT1-1 Cells, Mouse GT1-7 Cells, Rat HiB5 Cells, Rat Primary Hippocampal Neuron Cells, Rat SCN2.2 Cells, Rat F-11 Cells, Human SW-13 Cells, Human SV40 MES 13 Cells, Human Mesenchymal Stem Cells (hMSC), Human BGO1V Embryonic stem Cells, Human H9, Embryonic stem Cells, Mouse Embryonic stem Cells, Human adipose-derived stem cells (ADSC), Human Neural Stem Cells, Rat Neural Stem Cells, Fibroblast (iPSC)-Fibroblast, CD34+, Adipose, Derived Stem Cells, Adrenal Cortical Cells, Alpha Cells, Annulus Fibrosus Cells, Astrocytes, Beta Cells, Chondrocytes, Endothelial Cells, Epithelial Cells, Fibroblasts, Hair Cells, Hematopoietic Stem Cells, Immune Cells, Keratinocytes, Keratocytes, Melanocytes, Meningeal Cells, Mesangial Cells, Mesenchymal Stem Cells, Muscle Myoblasts, Muscle Sattellite Cells, Nucleus Pulposus Cells, Osteoblasts, Pericytes, Perineurial Cells, Schwann Cells, Skeletal Muscle Cells, Smooth Muscle Cells, Stellate Cells, Synoviocytes, Thymic Epithelial Cells, Trabecular and Meshwork Cells, Trophoblasts, Bone Marrow CD34+ Stem/Progenitor Cells, Bone Marrow Mononuclear Cells, Cord Blood Mononuclear Cells, Cord Blood CD34+ Stem/Progenitor Cells, Cord Blood CD34-Depleted MNC, Cord Blood CD3+ Pan T Cells, Cord Blood CD4+ Helper T Cells, Cord Blood CD8+ Cytotoxic T Cells, Cord Blood CD4+/CD45RA+ Naive T Cells, Cord Blood CD14+ Monocytes, Cord Blood CD56+ Natural Killer Cells, Cord Blood Plasma, Diseased Peripheral Blood CD19+/CD5+ B Cells, Diseased Bone Marrow CD19+/CD5+ B Cells, Diseased Bone Marrow MNC, Diseased Peripheral Blood MNC, Diseased Bone Marrow MNC, Diseased Peripheral Blood MNC, Diseased Peripheral Blood MNC, Diseased Peripheral Blood MNC, Diseased Peripheral Blood MNC, Diseased Peripheral Blood MNC, Diseased Bone Marrow MNC, Diseased Peripheral Blood MNC, Diseased Peripheral Blood Plasma, Mobilized Peripheral Blood Mononuclear Cells, Mobilized Peripheral Blood CD34+ Stem/Progenitor Cells, Mobilized Peripheral Blood CD14+ Monocytes, Bone Marrow Mesenchymal Stem/Stromal Cells, Diseased Peripheral Blood MNC, Peripheral Blood Monocyte-Derived Dendritic Cells, Peripheral Blood Monocyte-Derived Macrophages, Peripheral Blood Mononuclear Cells (PBMC), Peripheral Blood CD3+ Pan T Cells, Peripheral Blood CD4+ Helper T Cells, Peripheral Blood CD8+ Cytotoxic T Cells, Peripheral Blood CD4+/CD25+ Regulatory T Cells, Peripheral Blood CD4+/CD45RA+/CD25− Naive T Cells, Peripheral Blood CD8+/CD45RA+ Naive Cytotoxic T Cells, Peripheral Blood CD4+/CD45RO+ Memory T Cells, Peripheral Blood CD19+ B Cells, Peripheral Blood CD19+/IgD+Naive B Cells, Peripheral Blood CD14+ Monocytes, Peripheral Blood CD56+ Natural Killer Cells, Peripheral Blood Basophils, Peripheral Blood Eosinophils, Peripheral Blood Neutrophils, Peripheral Blood Plasma, Peripheral Blood Platelets, Peripheral Blood Mature Erythrocytes (RBC), Peripheral Blood CD34+ Stem/Progenitor Cells, Diseased Peripheral Blood MNC, Diseased Peripheral Blood MNC, Diseased Peripheral Blood MNC, Diseased Peripheral Blood MNC, Induced pluripotent cell (iPS cells), “True” embryonic stem cell (ES cells) derived from embryos, Embryonic stem cells made by somatic cell nuclear transfer (ntES cells), Embryonic stem cells from unfertilized eggs (parthenogenesis embryonic stem cells, or pES cells) Totipotent, Zygote, Spore, Morula,

Pluripotent, Embryonic stem cell, Callus, Multipotent cells, Progenitor cells, Endothelial stem cells, Hematopoietic stem cells, Mesenchymal stem cells, Neural stem cell, Neural Progenitor cells, Unipotent Precursor cells, Oligodendrocyte precursor cell, Myeloblast, Thymocyte, Meiocyte, Megakaryoblast, Promegakaryocyte, Melanoblast, Lymphoblast, Bone marrow, precursor cells, Normoblast, Angioblast (endothelial precursor cells), Myeloid precursor cells, Neural Stem Cells, Neural Porgenitor Cells, Neural Precursor Cells, Discovery, Intestinal enteroendocrine cells, K cell, L cell, I cell, G cell, Enterochromaffin cell, N cell, S cell, D cell, M cell, Gastric enteroendocrine cells, Pancreatic enteroendocrine cells, Alpha cells, Beta Cells, Delta Cells, PP cells, Epsilon Cells, Hepatocytes, Kupffer Cells, Stellate (Ito) Cells, Liver Sinusoidal Endothelial Cells, Neurons (unipolar, bipolar, multipolar, Golgi I and II, Anaxonic, peuodounipolar), Basket Cells, Betz Cells, Lugaro Cells, Medium spiny neurons, Purkinje Cells, Pyramidal cells, Renshaw cells, Unipolar brush cells, Granule Cells, Anterior Horn Cells, Spindle Cells, Salivary gland mucous cell (polysaccharide-rich secretion), Salivary gland number 1 (glycoprotein enzyme-rich secretion), Von Ebner's gland cell in tongue (washes taste buds), Mammary gland cell (milk secretion), Lacrimal gland cell (tear secretion), Ceruminous gland cell in ear (earwax secretion), Eccrine sweat glandering dark cell (glycoprotein secretion), Eccrine sweat gland clear cell (small molecule secretion), Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Insolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion, Paneth cell of small intestine (lysozyme secretion), Type II pneumocyte of lung (surfactant secretion), Club cell of lung, Anterior pituitary cells, Somatotropes, Lactotropes, Thyrotropes, Gonadotropes, Corticotropes, Intermediate pituitary cell, secreting melanocyte-stimulating hormone, Magnocellular neurosecretory cells, nonsecreting oxytocin, secreting vasopressin, Gut and respiratory tract cells, secreting serotonin, secreting endorphin, secreting, somatostatin, secreting gastrin, secreting secretin, nonsecreting cholecystokinin, secreting insulin, secreting glucagon, nonsecreting bombesin, Thyroid gland cells, Thyroid epithelial cell, Parafollicular cell, Parathyroid gland cells, Parathyroid chief cell, Oxyphil cell, Adrenal gland cells, Chromaffin cells, secreting steroid hormones (mineralocorticoids and gluco corticoids), Leydig cell of testes secreting testosterone, Theca interna cell of ovarian follicle secreting estrogen, Corpus luteum cell of ruptured ovarian follicle secreting progesterone, Granulosa lutein cells, Theca lutein cells, Juxtaglomerular cell (renin secretion), Macula densa cell of kidney, Peripolar cell of kidney, Mesangial cell of kidney, Pancreatic islets (islets of Langerhans), Alpha cells (secreting glucagon), Beta cells (secreting insulin and amylin), Delta cells (secreting somatostatin), PP cells (gamma cells) (secreting pancreatic polypeptide), Epsilon cells (secreting ghrelin), Erythrocyte (red blood cell), Megakaryocyte (platelet precursor), Monocyte (white blood cell), Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Hybridoma cell, Mast cell, Helper T cell, Suppressor T cell, Cytotoxic T cell, Natural killer T cell, B cell, Natural killer cell, Reticulocyte, Hematopoietic stem cells and committed progenitors for the blood and immune system (various types).

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### EXAMPLES

### General Protocol for Screening

A general protocol for screening to identify targets involved in cell differentiation includes (1) introducing into each of a plurality of stem cells a dCas protein linked to a transcription regulator and one or more guide RNAs; (2) isolating, from the plurality of stem cells, a viable cell that contains the dCas protein linked to the transcription regulator and at least one of the guide RNAs; (3) measuring gene expression in the viable cell or progeny thereof; and (4) correlating a change in gene expression in the viable cell or progeny thereof with one or more targets of the guide RNAs in the viable stem cell. Screening for targets involved in producing insulin-producing synthetic beta cells by human iPSC differentiation is an example.

In the following examples, the starting cell type is a human iPSC and a CRISPRa screen is used to differentiate the iPSCs to the target phenotype of an insulin-producing synthetic beta cell. It is understood that the methods disclosed may generally be applied to any starting cell type to produce any target phenotype and that any combination of gRNAs and effector domains to cause CRISPRa activation activity or CRISPRi inhibition activity may be employed. Preferably the transcription regulator under guidance of the dCas protein and one or more guide RNAs will cause differentiation of one of the plurality of stem cells into the viable cell or progeny thereof such that correlating the change in gene expression with the targets of the guide RNAs identifies loci to target by CRISPRa and/or CRISPRi to differentiate pluripotent stem cells into a target cell type. As the starting cells for screening, a dCas9-VPR stable iPSC cell line is created. Alternatively, any existing iPS cell line may be used.

1. iPSC Sample Preparation

Introducing the dCas protein linked to the transcription regulator into the stem cells may be done by delivering to the stem cells a vector that encodes a fusion protein comprising the dCas protein and the transcription regulator (e.g., a viral vector, a plasmid, or transposable element). Optionally, (e.g., where the vector uses a selection marker, and one can select for cells transformed by the vector) the cells are selected for transformation by the vector prior to introducing the one or more guide RNAs.

When a dCas9-VPR stable iPS cell line is first created, the dCas9-VPR complex can be constitutively expressed with a promoter non-silenced in stem cells, such as human elongation factor 1 alpha (HEF1α) or spleen focus-forming virus (SFFV), or can be inducible in its expression, such as a doxycycline-inducible Tet response element (TRE). The dCas9-VPR complex also contains a selection marker to isolate and enrich for cells with stable uptake of the dCas9-VPR complexes. The selection marker may be, for example, a fluorescent maker (GFP), a drug resistance marker (blasticidin), or any surface marker. For instance, a GFP marker gene may be attached downstream of a promoter sequence for a certain gene (i.e., a gene encoding the dCas9-VPR complex) to fluorescently report promoter activity indicating expression of that gene.

This dCas9-VPR complex is introduced into iPSCs using viral vectors (e.g., lentiviral) or transposable elements (piggyBac). Cells that successfully integrated the dCas9-VPR complex are selected by FACS (GFP selection marker) or drug resistance (blasticidin). Targeted genomic DNA (gDNA) and/or allele-specific qPCR is used to confirm successful integration of the dCas9-VPR complex. qPCR gene expression analysis is performed to confirm iPSC pluripotency and trilineage potential. Table 1 below shows gene markers for pluripotency and trilineage potential.

TABLE 1
Gene markers for pluripotency and trilineage potential

| Gene | Function |
|---|---|
| POU5F1 | Pluripotency |
| SOX2 | Pluripotency |
| NANOG | Pluripotency |
| PAX6 | Ectoderm |
| NES | Ectoderm |
| SOX1 | Ectoderm |
| SOX17 | Endoderm |
| GATA4 | Endoderm |
| FOXA2 | Endoderm |
| T | Mesoderm |
| HAND1 | Mesoderm |
| TBX6 | Mesoderm |

Next, either a library of barcoded sgRNAs targeting genome-wide promoters with 4-30 sgRNAs per gene or a specified subset of genes is delivered to the selected cells. Delivery methods include transient transfection (e.g., lipofection, electroporation, or NanoLaze) or stable delivery by virus (e.g., lentiviral, sendai). The cells are then enriched for those successfully receiving sgRNAs by FACS or drug selection.

The stem cells may be delivered into reaction vessels (e.g., wells of a plate) such that each reaction vessel receives, on average, between 0 and 2 of the stem cells. The guide RNAs may have targeting portions that map to promoter regions of genes associated with a desired phenotype or trait. Each reaction vessel may get guide RNAs that target either one or a plurality of genes associated with the desired phenotype or trait. For each gene that is targeted, between one and 40 distinct guide RNAs may be provided. Preferably, for each guide RNA that is delivered, between about 1 and about 20 copies of the guide RNA are delivered.

In one approach, genes are targeted individually in high throughput array format (96-well, 384-well), where activation of a single cell is desired per well. In a single gene activation approach, individual sgRNAs per well or 4-10 pooled sgRNAs per gene per well are used. In another approach, all sgRNAs are pooled and delivered to a whole population of cells. When a viral vector is employed, a multiplicity of infection (MOI) is used, where each cell statistically receives either a single sgRNA or the necessary number of pooled sgRNAs. Targeted gDNA sequencing is used to confirm MOI after transduction.

In an optional workflow, when the starting cells for screening are from an existing iPS cell line, recombinant dCas9-VPR ribonucleoproteins (RNPs) complexed with the barcoded sgRNA library may be directly delivered to the iPSCs either in pooled or individually arrayed format. Because RNPs are transient (24-72 hours), it is necessary to perform repeat deliveries. However, this approach provides the advantage of temporal control in targeting multiple genes across a time frame (e.g., a few days to a few weeks) to determine the effects of their collective input on producing the desired target phenotype. In the pooled format, the dosage of RNPs may be titered so that each cell statistically receives more or less complex combinations of sgRNAs. In this approach, any iPS cell line can be used as the starting cell type for screening.

### 2. Analysis and Target Identification

After the sgRNA library components (i.e., the barcoded sgRNAs) are delivered to cells, two subsequent approaches can be taken for selection and analysis: a ‘biased’ approach and/or an ‘unbiased’ approach. The ‘biased’ approach involves selecting for cells that are viable and demonstrate the target phenotype. In contrast, the ‘unbiased’ approach involves only selecting for cells that are viable. The protocol includes isolating a viable cell by selecting a cell that exhibits a desired trait. Selecting the cell that exhibits the desired trait may include staining the plurality of stem cells with a marker for the desired trait, and sorting the cells on a fluorescence-activated cell sorting instrument.

In the ‘biased’ approach, desired traits are selected for in cells carrying the dCas9-VPR complexes and sgRNAs. For the purpose of generating insulin-producing synthetic beta cells, iPSCs undergo staining with markers for both viability and desired traits (C-peptide+/Insulin+, Chromogranin A+, Nkx6.1+, Glucagon-, Somatostatin-) conjugated to fluorescent probes for subsequent FACS at defined time points (e.g., weekly intervals from 1-6 weeks). The cells may be sorted as whole populations for analysis or as single cells for isogenic expansion or single-cell analysis. In contrast, in the unbiased approach, cells undergo FACS with markers only for viability to isolate single cells for isogenic expansion or single-cell analysis. Here, the desired trait includes a specified differentiated cell type and the marker includes a protein expressed by the differentiated cell type (e.g., the presence of C-peptide, Insulin, Chromogranin A, and Nkx6.1, and the absence of Glucagon and Somatostatin).

### 3. Measuring Fold-Change Effects on Gene Expression

Measuring gene expression in the viable cell or progeny thereof may include one or more of quantifying expression levels via RNA-Seq or evaluating DNA-protein interaction via chromatin immunoprecipitation and DNA sequencing (ChIP-seq). Here, determining fold-change in expression level of a transcript associated with the marker involves normalizing read counts from the measuring against control read counts.

After cells have been isolated as single-cells, single-cell NGS, including RNA sequencing (RNA-seq) or chromatin immunoprecipitation sequencing (ChIP-seq) is performed to produce sequence reads for the gRNAs. In the case where one barcoded sgRNA is present within a single cell, single-cell NGS can be used to directly characterize the transcriptomic or epigenomic effects of the dCas9-VPR complex targeted by that specific sgRNA. In the case where multiple barcoded sgRNAs targeting the same or different genes are present within a single cell, their collective genetic interactions can be used to identify networks of factors that are important for directing cell fate and function.

### 4. Correlating Change to Guide Targets

When the ‘biased’ approach is used to select for insulin-producing cells, NGS sequence data is de-multiplexed using unique index reads and barcoded sgRNA counts were determined by only perfect-match sequencing reads. The sgRNA fold change resulting from screening conditions is calculated by dividing normalized counts in the test conditions by controls, followed by taking the base-2 logarithm. If a low percent of functional sgRNAs is expected per targeted locus, a weighted sum method can be used. Preferably, the guide RNAs are barcoded, and the method further comprises using a computer system to analyze sequence data to determine the fold-change for the transcript and correlate, using barcode sequences in the sequence data, the fold-change for the transcript the one or more targets of the guide RNAs in the viable stem cell.

The false discovery rate (FDR) of candidate loci was determined by taking the weighted sum for 10 randomly selected non-targeting sgRNAs in the library to estimate the P-value for each targeted locus. A threshold based on an FDR of 0.05 (Benjamini-Hochberg) was selected to correspond to a pre-determined P-value. A set of candidate loci with sufficiently low P-values are then selected based on an average ranking between replicates. Further analysis of differential expression between cells receiving sgRNAs for one gene vs. another and vs. non-targeting controls is used to perform difference of difference analyses to identify additional factors within gene expression networks that contribute to producing the target phenotype for further testing.

### 5. An Unbiased Approach

When the ‘unbiased’ approach is used to select for insulin-producing cells, machine learning on the NGS data produced is applied to predict combinations of genetic loci targeted by the complexes which cause alterations in transcriptional networks to produce the target phenotypes, for example, insulin production. Training data for the machine learning can include NGS data produced by either or both of the ‘biased’ and ‘unbiased’ approaches, along with other publicly-available sequencing data from various stages of the natural development of insulin-producing cells or the differentiation of pluripotent stem cells to insulin-producing synthetic beta cells. Training and test activity score sets are divided into sets of genes and training set parameters are transformed based on their distributions. Binning parameters are applied to collapse sparse data points into consolidated bins. Features are then split into individual parameters for each bin and sgRNAs or targets were assigned as “1” if the value fell within the bin and “0” if not. Other parameters may be linearized accordingly, then z-standardized and fit with elastic net linear regression.

6. Combinatorial Approaches—CRISPRa/i with Other Factors

It is possible that the expression of certain genes are not activated/inhibited by CRISPRa/I to mediate differentiation/emergence of a phenotype in an effective manner. Thus, activation of a specific factor may be replaced or enhanced by over-expression of that factor via the other approaches (integrated via virus/PiggyBac, delivered via DNA/RNA/protein, etc). To get sufficient differentiation to a desired phenotype, it may be useful to target some genes with CRISPRa/i while simultaneously or in sequence expressing other genes/factors via an integrated PiggyBac. For background, see Balboa, 2015, Conditionally stabilized dCas9 activator for controlling gene expression in human cell reprogramming and differentiation, Stell Cell Reprots 5:448-459, incorporated by reference. CRISPRa may be used to inducibly activate one or more target genes, followed by inducible expression of the transcription factor, to differentiate pluripotent stem cells to preferred cell types. Methods of the disclosure include combinations of CRISPRa/i+/−transcription factors (TFs) to mediate differentiation.

In some embodiments, a transcription regulator under guidance of the dCas protein and one or more guide RNAs results in differentiation of one of the plurality of stem cells into the viable cell or progeny thereof in combination with initiating expression of, or introducing, one or more additional gene products to promote differentiation of the one of the plurality of stem cells into the viable cell or progeny thereof. Expression of at least one of the additional gene products may be initiated by one selected from the group consisting of: introducing a corresponding gene using a PiggyBac transposon; introducing a corresponding gene via a plasmid or viral vector; introducing an mRNA encoding the gene product. Additionally or alternatively, additional gene products may be introduced as proteins. The gene product may be, for example, a transcription factor such that the transcription factor and the transcription regulator under guidance of the dCas protein and one or more guide RNAs results in differentiation of the stem cells into, for example a beta islet cell.

### 7. Timing of Factors Being Turned On/Off.

Embodiments of the disclosure include temporal control of CRISPRa/I+/−TFs. For example, in order to get to the desired synthetic glucose-responsive insulin-secreting synthetic beta cell, CRISPRa/i may be used against a few targets for the first 2-3 days, followed by CRISPRa/I against some of the same or different genes+/−other genes expressed via PiggyBac TF for another # of days, then CRISPRa/I against some similar or other targets for the remaining # of days of differentiation. Thus, the transcription regulator under guidance of the dCas protein and one or more guide RNAs may result in differentiation of one of the plurality of stem cells when guide RNAs are introduced into at least one of the plurality of stem cells in a temporal sequence. The temporal sequence may include the introduction of a first set of one or more guide RNAs during a first period comprising one or more hours or days followed by introduction a second set of one or more guide RNAs during a second period comprising one or more hours or days. Optionally, the first set of one or more guide RNAs and the second set of one or more guide RNAs comprise wholly different guide RNAs and/or the first period and the second period do or do not overlap in time. Certain embodiments of methods of the disclosure involve using CRISPRa/i against a first set of targets during the first period, the first period comprising at least two days, and using CRISPRa/I against a second set of targets during the second period to differentiate the one of the plurality of stem cells into a glucose-responsive insulin-secreting beta cell.

### Islet-Beta-Cell-Like Specification of Stem Cells Via Directed Genetic Modulation

Embodiments of the present invention are directed to methods for the targeted specification of stem cells to beta cell-like cells of the pancreatic islet via directed genetic modulation. The beta cell-like cells are suitable for use in various applications, including cell therapy. Certain embodiments use laser-activated intracellular delivery of CRISPR-Cas systems for genome engineering and altering gene expression in induced pluripotent stem cells (iPSCs). Targeted genetic modulation of key regulatory factors in the pluripotent stem cell state allows the directed differentiation of stem cells to functional beta-like cells found in the pancreatic islets that secrete insulin and respond to glucose.

As a long-established field, cadaveric islet transplantation provides a promising approach to treating and even potentially providing a functional cure for insulin-dependent diabetes. Long-term follow up of the Edmonton Protocol has demonstrated that transplantation of sufficient quantities of mixed population islets harvested from cadaveric donors can yield partial, if not complete, independence from insulin use. However, this field is limited by a clinical supply of islet donors that could be remedied via robust production of beta-like cells from either autologous or allogeneic stem cell sources. Moreover, the production of cell populations in which the representation of beta cells relative to other cell types (e.g. alpha, delta, etc.) is maximized may lead to improved clinical efficacy and/or duration of response.

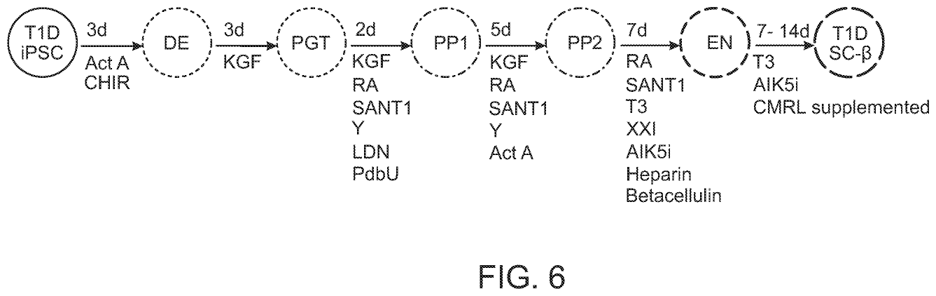

Historically, the generation of beta-like cells has relied predominantly on the application of exogenous factors in media to sequentially differentiate stem cells into functional insulin-secreting cells. Such approaches use small molecules, signaling factors, hormones, and other soluble media components to drive step-by-step differentiation of stem and progenitor cells, such as shown in FIG. 6. Typically, pluripotent stem cells are first driven to definitive endoderm (DE) for 3 days with Activin A and the GSK3beta inhibitor CHIR99021. Then, primitive gut tube (PGT) for 3 days with keratinocyte growth factor (KGF), pancreatic progenitor cells (PP1) for 2 days (KGF, retinoic acid (RA), sonic hedgehog pathway antagonist (SANT1), Y, LDN, PdbU), later pancreatic progenitor cells (PP2) for 5 days (KGF, RA, SANT1, Y, Act A), endocrine cells (EN) for 7 days (RA, SANT1, T3, XXI, Alk5i, Heparin, Betacellulin), and final maturation for 7-14 days (T3, Alk5i, CMRL). Conversely, the present invention is directed to methods of directed differentiation of pluripotent stem cells or other intermediate progenitor states to insulin-secreting beta-like cells using targeted genetic modulation, thereby bypassing these stages and durations of lineage specification.

In the present invention, examples of stem cells include, but are not limited to, induced pluripotent stem cells (iPSCs), embryonic stem cells (ESCs), epiblast stem cells (epiSCs), and intermediate progenitor states derived from human, mouse, rat, and other mammalian species. Genetic targets are those defined as most likely to yield beta cell-specification of cell fate and include MafA, NeuroD1, Neurog3, Nkx2.2, Nkx6.1, Pax4, Pdx1, and Six2, as shown below in Table 2.

TABLE 2
Beta Cell-Specifying Factors

| Gene | Consensus Coding Sequence ID |
|---|---|
| MAFA | CCDS34955.1 (SEQ ID NO: 1) |
| NEUROD1 | CCDS2283.1 (SEQ ID NO: 2) |
| NEUROG3 | CCDS31212.1 (SEQ ID NO: 3) |
| NKX2-2 | CCDS13145.1 (SEQ ID NO: 4) |
| NKX6-1 | CCDS3607.1 (SEQ ID NO: 5) |
| PAX4 | CCDS5797.1 (SEQ ID NO: 6) |
| PDX1 | CCDS9327.1 (SEQ ID NO: 7) |
| SIX2 | CCDS1822.1 (SEQ ID NO: 8) |

Any suitable approach may be used to modulate the activity of the genes. In an example, an approach for modulating activity is direct expression by factor introduction. Another example approach is over-/under-expression by CRISPR activators/inhibitors (CRISPRa/i). In an embodiment, the present invention provides approaches and techniques at the DNA level used to achieve the direct expression of the desired gene. In another embodiment, approaches and techniques are provided at the RNA and/or protein level used to achieve the direct expression of the desired gene.

For example, at the DNA level, the beta cell-specifying Consensus Coding Sequences (Table 2) are cloned or synthesized into either lentiviral or piggyBac backbone vectors containing constitutive or drug-inducible promoters, along with fluorescent and/or drug selection markers to produce stable cell lines. Via the lentiviral approach, the lentiviral backbone vector containing the desired insert is packaged by a lentivirus-producing cell line (e.g. HEK293T/FT), and virus is collected, purified, stored, then used for transduction of the target stem cell line. After selection to generate stable cell lines, drug induction leads to controlled overexpression of the desired factor. Via the piggyBac approach, the gene inserts are cloned into a piggyBac compatible backbone vector (e.g System Biosciences PBQM812A-1) and transfected with the transposon-expressing vector (i.e. System Biosciences PB210PA-1) into the target cell line. After selection to generate stable cell lines, drug induction leads to controlled overexpression of the desired factor.

In embodiments providing approaches at the RNA and protein levels, the in vitro transcribed mRNA or synthetically modified RNA coding for the desired factors and/or the purified proteins themselves are delivered directly to the target cells. Examples of delivery methods include using nanoparticle-based transfection (e.g. lipofection), electroporation (e.g. nucleofection), laser-activation of substrates (i.e. NanoLaze), or other physical delivery methods. Repetitive delivery with the same or different cargo permutations on the same or subsequent days may be necessary to yield differentiation via this approach.

In certain embodiments, laser-activated intracellular delivery of CRISPR-Cas systems is used for genome engineering and altering gene expression in induced pluripotent stem cells (iPSCs). In an embodiment, in order to drive the overexpression or inhibition of endogenous loci, CRISPRa/i is used with one or more single guide RNAs (sgRNAs) that target within −300 to +0 base pairs of the transcription start site (TSS) per target gene (Table 3, shown below) in stable cell lines or ribonucleoprotein (RNP) complexes.

In an example, stable cell lines expressing the dCas9-VPR, or other suitable CRISPRa constructs, are generated via lentiviral or piggyBac incorporation into the genome with constitutive or drug-inducible promoters, along with fluorescent and/or drug selection markers. In this instance, sgRNAs may be delivered to these stable cell lines with nanoparticle-based transfection (e.g. lipofection), electroporation (e.g. nucleofection), laser-activation of substrates (i.e. NanoLaze), or other physical delivery methods. Repetitive delivery with the same or different sgRNA permutations on the same or subsequent days may be necessary to yield differentiation.

In an example, RNP complexes are delivered directly to cell lines by nanoparticle-based transfection (e.g. lipofection), electroporation (e.g. nucleofection), laser-activation of substrates (i.e. NanoLaze), or other physical delivery methods. Repetitive delivery with the same or different RNP permutations on the same or subsequent days may be necessary to yield differentiation.

Notably, regarding the CRISPRa approach, a recent paper has shown that CRISPRa targeting Pdx1 in vivo is sufficient to transdifferentiate liver cells into insulin-producing cells in a mouse model, as shown in In Vivo Target Gene Activation via CRISPR/Cas9-Mediated Trans-epigenetic Modulation, Liao et al., Cell (2017), the content of which is incorporated herein by reference in its entirety.

Methods of the present invention provide for the identification, discrimination of monohormonal vs. polyhormonal, and characterization of the produced cells using suitable techniques regardless of the method used to drive specification of stem cells into insulin-producing beta-like cells. In an example of a suitable technique, cellular RNA is collected and analyzed by qRT-PCR, microarray, and/or next-generation sequencing for differential expression of beta cell-specifying genes in Table 2, along with the insulin (INS) and glucagon (GCG) genes. In another example of a suitable technique, cells are fixed and stained for expression of insulin (c-peptide), glucagon, and/or chromogranin A. In an example of a suitable technique, cells are stimulated to secrete insulin via escalating doses of glucose-supplemented media.

Reproducible beta-like cell populations derived via the method that best optimizes the quantity and purity of beta cells from stem cells, as determined by the aforementioned analytical techniques, are next validated for in vivo functionality through transplantation in mouse models. In the first instance, beta-like cells are transplanted into normoglycemic mice, which undergo periodic fasting blood glucose and glucose challenge testing to elicit insulin responsiveness, followed by sacrifice and explant analysis for maintenance of cell identity at the end of the animal trial period. In the second instance, beta-like cells are transplanted into hyperglycemic/non-obese diabetic (NOD) mice and followed as above; it is anticipated that beta cell supplementation in hyperglycemic mice contributes to glycemic normalization through glucose-responsive insulin production, resulting in potential extension of life.

TABLE 3
Target genes and sgRNA sequences

| Gene | sgRNA Sequences |
|---|---|
| MAFA | CTGGGCTCTGAGTTGCCATG |
| | CTCCTGCGGGAAACAGCTGT |
| | GCCCTCTGGTGGCCATCACG |
| | AGGGACGGGCCGCCGGCTAG |
| | CCATGGGGATAAGCAAATGA |
| | ACGGCAGTTGTCCCCTGAGG |
| | GCCCAGCTGTCAATCTCCTG |
| | GCTCTATAAAGGGGCGCGCG |
| | CCCAGCTGTCAATCTCCTGC |
| | TTCCCACAGCTGTTTCCCGC |
| | GCCCGTGCAGTGCCCCGTGA |
| | GAGACGGCAGTTGTCCCCTG |
| | GCCATCACGGGGCACTGCAC |
| | GGGACAACTGCCGTCTCCAG |
| | CCCGCAGGAGATTGACAGCT |
| | TCTCCTGCGGGAAACAGCTG |
| | CCCTCATTTGCTTATCCCCA |
| | GCTGGGCTCTGAGTTGCCAT |
| | GCGCGCCCCTTTATAGAGCC |
| | TCCCGCAGGAGATTGACAGC |
| | CTGCCCTCTCCTCTAGCCGG |
| | CAGAGGGCGCGCCGCCTCAG |
| | CTGTGGGAAGAGGTAGGGAC |
| | AACAGCTGTGGGAAGAGGTA |
| | GGGGACAACTGCCGTCTCCA |
| | AGGCACCCGGCTCTATAAAG |
| | GCCGCCGGGTGTGGGAGCTG |
| | CGGGGCACTGCACGGGCAGA |
| | GCCCTGCCCTCTCCTCTAGC |
| | CTTTATAGAGCCGGGTGCCT |
| | GGCCATCACGGGGCACTGCA |
| | GAGAGGGCAGGGCCCTCTGG |
| | ATAAGCAAATGAGGGCGGCG |
| | GATAAGCAAATGAGGGCGGC |
| | CCTTTATAGAGCCGGGTGCC |
| | GGCCCTCTGGTGGCCATCAC |
| | CCAGGCACCCGGCTCTATAA |
| | GGATAAGCAAATGAGGGCGG |
| | CGGGAAACAGCTGTGGGAAG |
| | TGGGGATAAGCAAATGAGGG |
| | GCAGAGGGCGCGCCGCCTCA |
| | AGGGGACAACTGCCGTCTCC |
| | GGGCCGCCGGCTAGAGGAGA |
| | GGAGCTGAGGCCCCTGGAGA |
| | GCCATGGGGATAAGCAAATG |
| | AAGAGGTAGGGACGGGCCGC |
| | GCTGTGGGAAGAGGTAGGGA |
| | AAACAGCTGTGGGAAGAGGT |
| | GCCGGCTAGAGGAGAGGGCA |
| | TGCCCCGTGATGGCCACCAG |
| | CGCGCGCCCCTTTATAGAGC |
| | AGCTGGGCTCTGAGTTGCCA |
| | GCCCCGTGATGGCCACCAGA |
| | CGCCGGCTAGAGGAGAGGGC |
| | CAGGCACCCGGCTCTATAAA |
| | GGGGGCGCGCGCCCCTCCCG |
| | TGGGGCTCCGATTGGCCCGG |
| | GGCGGGGCTGGGGCTCCGAT |
| | GAGGAGAGGGCAGGGCCCTC |
| | CCTGGGCCCGCCTCCCCGGG |
| | TGGGCCCGCCTCCCCGGGAG |
| | GGCAGAGGGCGCGCCGCCTC |
| | GCTGGGGCTCCGATTGGCCC |
| | CTGGGCCCGCCTCCCCGGGA |
| | CGGGAGGGGCGCGCGCCCCC |
| | GGCTGGGGCTCCGATTGGCC |
| | ACGGGGCACTGCACGGGCAG |
| | GGCGCGCGCCCCTCCCGGGG |
| | AAATGAGGGCGGCGGGGCTG |
| | GTGCCTGGGCCCGCCTCCCC |
| | GCGCGCGCCCCCGGGCCAAT |
| | GGTGTGGGAGCTGAGGCCCC |
| | CGCGCCCCTCCCGGGGAGGC |
| | GGTGCCTGGGCCCGCCTCCC |
| | CAAATGAGGGCGGCGGGGCT |
| | CGGGGGCGCGCGCCCCTCCC |
| | GGGCCCTCTGGTGGCCATCA |
| | CCGGGGGCGCGCGCCCCTCC |
| | GCGCGCCCCTCCCGGGGAGG |
| | CCGGGAGGGGCGCGCGCCCC |
| | GCAAATGAGGGCGGCGGGGC |
| | CTGGGGCTCCGATTGGCCCG |
| | GGGAGGCGGGCCCAGGCACC |
| | CCTCCCGGGGAGGCGGGCCC |
| | (SEQ ID NO: 9) |
| NEUROD1 | TGGGCGAATTCCTCGTGTCG |
| | CCAGTTAGTGATGCTAAGCG |
| | ATATAACCTGAGCGCCCGCG |
| | TTAGTGATGCTAAGCGCGGG |
| | CAGTTAGTGATGCTAAGCGC |
| | GAAGACCATATGGCGCATGC |
| | CCTGCTAGCCCCTCAGCTAG |
| | AGACCATATGGCGCATGCCG |
| | GCCCGCGCGGCCACGACACG |
| | CGGGAGACGAGCAAGGCGTG |
| | ATACAAATGGGCAGGTCACG |
| | TTCCTCGTGTCGTGGCCGCG |
| | GGGGAGCGGTTGTCGGAGGA |
| | AGTGATGCTAAGCGCGGGCG |
| | TCCAGGCTCTTGGCTGGACC |
| | GGGCGGGGCCGCTAGCTGAG |
| | GCTGGACCGGGAAGACCATA |
| | CGTGGTTCCAGGCTCTTGGC |
| | CGCGCTTAGCATCACTAACT |
| | TAGTGATGCTAAGCGCGGGC |
| | GCAAGGCGTGGGGAGAAGTG |
| | AGGAGGGCGGGAGACGAGCA |
| | AAGACCATATGGCGCATGCC |
| | CCATATGGCGCATGCCGGGG |
| | AGGGTGAGGGGAGCGGTTGT |
| | AGCAAGGCGTGGGGAGAAGT |
| | GTGAGGGGAGCGGTTGTCGG |
| | TCCCGGTCCAGCCAAGAGCC |
| | GCGGGAGACGAGCAAGGCGT |
| | AGAACGGGGAGCGCACAGCC |
| | AGCGGTTGTCGGAGGAGGGC |
| | CATGCGCCATATGGTCTTCC |
| | CCGCTAGCTGAGGGGCTAGC |
| | GTCACGTGGTTCCAGGCTCT |
| | TGGACGCGTGCGCAGGCGTC |
| | CGCCCCTCCTCCTTCCTCCC |
| | GCGCATGCCGGGGAGGAAGG |
| | CCTCCCCGGCATGCGCCATA |
| | TCCTCGTGTCGTGGCCGCGC |
| | ATGCCGGGGAGGAAGGAGGA |
| | GGGGCGGGGGTAGGGGTGGA |
| | GAGCAAGGCGTGGGGAGAAG |
| | CACAGCCTGGACGCGTGCGC |
| | ATGGCGCATGCCGGGGAGGA |
| | CCGCGCTTAGCATCACTAAC |
| | AGGCGTGGGGAGAAGTGGGG |
| | GAGCGGTTGTCGGAGGAGGG |
| | TGGGCAGGTCACGTGGTTCC |
| | GTGGGGAGAAGTGGGGAGGA |
| | TGCCGGGGAGGAAGGAGGAG |
| | GGCGGGAGACGAGCAAGGCG |
| | AGGGGAGCGGTTGTCGGAGG |
| | TCGTGGCCGCGCGGGCGCTC |
| | CGGGCGGGGCCGCTAGCTGA |
| | GGAGGAAGGAGGAGGGGCGG |
| | TGACGCCTGCGCACGCGTCC |
| | TGGGGAGAAGTGGGGAGGAG |
| | TTCCAGGCTCTTGGCTGGAC |
| | GTGGGGAGGAGGGGAGAACG |
| | GGGTAGGGGTGGAGGGTGAG |
| | GCGGGCGGGGCCGCTAGCTG |
| | GGGGTAGGGGTGGAGGGTGA |
| | AAGGAGGAGGGGCGGGGGTA |
| | GGGGTGGAGGGTGAGGGGAG |
| | GGGAGGAAGGAGGAGGGGCG |
| | AGGAGGAGGGGCGGGGGTAG |
| | GAAGGAGGAGGGGCGGGGGT |
| | AAGTGGGGAGGAGGGGAGAA |
| | GGGGAGGAAGGAGGAGGGGC |
| | CGGGGAGGAAGGAGGAGGGG |
| | CATGCCGGGGAGGAAGGAGG |
| | AGTGGGGAGGAGGGGAGAAC |
| | GGGGGTAGGGGTGGAGGGTG |
| | CGTGGGGAGAAGTGGGGAGG |
| | AGGAGGGGCGGGGGTAGGGG |
| | AGGGGCGGGGGTAGGGGTGG |
| | (SEQ ID NO: 10) |
| NEUROG3 | TCTGTTTGCTCTCTCGAGGG |
| | GAAGCAGATAAAGCGTGCCA |
| | CCAGTGAGAAGAGCCTCGTG |
| | AAGCAGATAAAGCGTGCCAA |
| | GGCCTGACCAGAGCCACACG |
| | TTGAGGAACCGAGAGTTGCT |
| | TCTGGTCAGGCCACCTCAGA |
| | GCAGCAAGTCGTGTGCCCCT |
| | GGATTCCGGACAAAGGGCCG |
| | CCTCGAGAGAGCAAACAGAG |
| | AGCAGATAAAGCGTGCCAAG |
| | CACAGCTGGATTCCGGACAA |
| | ACAGCTGGATTCCGGACAAA |
| | AGGAGCAAAGCCGTCTGAGG |
| | CGGAATCCAGCTGTGCCCTG |
| | GAGGCTCTTCTCACTGGGCG |
| | GAATCCAGCTGTGCCCTGCG |
| | CCACACGAGGCTCTTCTCAC |
| | CACACGAGGCTCTTCTCACT |
| | AGCCTCGTGTGGCTCTGGTC |
| | CCCGACCCCGGCCCTTTGTC |
| | AGAAGAGCCTCGTGTGGCTC |
| | ACGCTTTATCTGCTTCGCCC |
| | CTGTTTGCTCTCTCGAGGGC |
| | CCGCTCTGTTTGCTCTCTCG |
| | CAATCAGCGCCGGGGCCCTG |
| | CGCTCTGTTTGCTCTCTCGA |
| | TGGATTCCGGACAAAGGGCC |
| | AATCCAGCTGTGCCCTGCGG |
| | CGCAGGGCACAGCTGGATTC |
| | CTGGATTCCGGACAAAGGGC |
| | GGAATCCAGCTGTGCCCTGC |
| | CACGCTTTATCTGCTTCGCC |
| | CCACCGGCCAATCAGCGCCG |
| | GCTAGGAGCAAAGCCGTCTG |
| | TCACTGGGCGAGGCTCTTTG |
| | CAGCCGGGCAGGCACGCTCC |
| | CCGGCTGCTGCCCGCGCCAC |
| | TCCGGACAAAGGGCCGGGGT |
| | GGGGGAGGAGCGGGCTCGCG |
| | GAGCCCGCTCCTCCCCCGCA |
| | CCGGACAAAGGGCCGGGGTC |
| | TTTGAGGAACCGAGAGTTGC |
| | TTATCTGCTTCGCCCGGGCC |
| | CCCCGGCGCTGATTGGCCGG |
| | GGCAGGCACGCTCCTGGCCC |
| | GGGCCAGGAGCGTGCCTGCC |
| | TGTGCCCTGCGGGGGAGGAG |
| | CCTCCCCCGCAGGGCACAGC |
| | GCTCGCGTGGCGCGGCCCCA |
| | AGGAGCGGGCTCGCGTGGCG |
| | TGCTCTCTCGAGGGCGGGCT |
| | CCCAGGGCCCCGGCGCTGAT |
| | CGGTGGCGCGGGCAGCAGCC |
| | GCGCTGATTGGCCGGTGGCG |
| | GCCAATCAGCGCCGGGGCCC |
| | GGACAAAGGGCCGGGGTCGG |
| | GGGCCCCGGCGCTGATTGGC |
| | GGCTGGGTCCCAGCAACTCT |
| | CCAGCTGTGCCCTGCGGGGG |
| | CGGACAAAGGGCCGGGGTCG |
| | GGGCAGGCACGCTCCTGGCC |
| | CGCTGATTGGCCGGTGGCGC |
| | GCGCTCCCCTCCCCCGACCC |
| | AAAGGGCCGGGGTCGGGGGA |
| | GCCACCGGCCAATCAGCGCC |
| | CGCCACCGGCCAATCAGCGC |
| | CCAATCAGCGCCGGGGCCCT |
| | CGAGCCCGCTCCTCCCCCGC |
| | GGCGCGGGCAGCAGCCGGGC |
| | GTGCCCTGCGGGGGAGGAGC |
| | TTGCTCTCTCGAGGGCGGGC |
| | GTGGCGCGGCCCCAGGGCCC |
| | CCGGTGGCGCGGGCAGCAGC |
| | AAGGGCCGGGGTCGGGGGAG |
| | CAAAGGGCCGGGGTCGGGGG |
| | GGCTCGCGTGGCGCGGCCCC |
| | (SEQ ID NO: 11) |
| NKX2-2 | TCCCAAGACCCGCCCACACG |
| | CGATCAGTCCATATAAGGCT |
| | TGGGCTCCACTCACGAACCT |
| | AAGAGACATTAAAAACGCAA |
| | CCTTATATGGACTGATCGCT |
| | TGGACTGATCGCTCGGGCAA |
| | CTTATATGGACTGATCGCTC |
| | CGCCTCCCCAGGTTCGTGAG |
| | GGGCTCCACTCACGAACCTG |
| | GCGATCAGTCCATATAAGGC |
| | TATTTGCAGATGTGAAATTG |
| | CTGGGCTCCACTCACGAACC |
| | GGCGGGTCTTGGGAGTCAAG |
| | CTCCACTCACGAACCTGGGG |
| | ATTTGCAGATGTGAAATTGT |
| | ATCTTGCTCTAGAGGGCCGT |
| | GACATTAAAAACGCAAAGGT |
| | ACGCAAAGGTTGGCCACGTG |
| | CACCTCTCATCTTGCTCTAG |
| | CTCACGAACCTGGGGAGGCG |
| | AAGGTTGGCCACGTGTGGGC |
| | AAAGGTTGGCCACGTGTGGG |
| | CCGAGCGATCAGTCCATATA |
| | ATGTGAAATTGTGGGTTTTG |
| | GCTCTAGAGGGCCGTTGGCT |
| | CGCAAAGGTTGGCCACGTGT |
| | GGAGAAGGGTGGAAAAAAGG |
| | AGGGCCGTTGGCTGGGAGCG |
| | GCCACGTGTGGGCGGGTCTT |
| | CACTCACGAACCTGGGGAGG |
| | GAGGGGGAGAAAGAGAGGGA |
| | GGGAGGGAAAGAAAGAGGGA |
| | AGCTCCGCGCTCCCAGCCAA |
| | ACCTCTCATCTTGCTCTAGA |
| | GAGAGGGAGCGGGAGAAGGG |
| | GATGTGAAATTGTGGGTTTT |
| | AGGGAGGGAGGGAAAGAAAG |
| | AGATGTGAAATTGTGGGTTT |
| | GAGTGGAGCCCAGCCTTATA |
| | GGGAGAAGGGTGGAAAAAAG |
| | ACTCACGAACCTGGGGAGGC |
| | GGGAGAAAGAGAGGGAGGGA |
| | GGCCACGTGTGGGCGGGTCT |
| | TGCTCTAGAGGGCCGTTGGC |
| | AACCTGGGGAGGCGGGGAGA |
| | GGGAGGGAGGGAAAGAAAGA |
| | GCTGGTGGCGAGGAAAAAAT |
| | GGGAGAGGGGGAGAAAGAGA |
| | GGCTGGTGGCGAGGAAAAAA |
| | ACCTGGGGAGGCGGGGAGAG |
| | CGGGAGAAGGGTGGAAAAAA |
| | AGAGGGGGAGAAAGAGAGGG |
| | GGGAAAGAAAGAGGGAGGGA |
| | GCGGGAGAAGGGTGGAAAAA |
| | AGGGAGGGAAAGAAAGAGGG |
| | CGAGAGAGGGAGCGGGAGAA |
| | CCCCCTCTCCCCGCCTCCCC |
| | GGGGAGAGGGGGAGAAAGAG |
| | GGGGAGAAAGAGAGGGAGGG |
| | GAAAGAAAGAGGGAGGGAGG |
| | CCTGGGGAGGCGGGGAGAGG |
| | GGGGGAGCGAGAGAGGGAGC |
| | GAACCTGGGGAGGCGGGGAG |
| | AGGGAAAGAAAGAGGGAGGG |
| | GAGGGAGGGGGAGCGAGAGA |
| | GCGAGAGAGGGAGCGGGAGA |
| | AGGGGGAGCGAGAGAGGGAG |
| | GGAAAGAAAGAGGGAGGGAG |
| | GGAGGGAGGGGGAGCGAGAG |
| | (SEQ ID NO: 12) |
| NKX6-1 | CAAGGCTACGGTCTCCGGCG |
| | GAGACCGTAGCCTTGCAGCG |
| | CTAACATCCCACGGCCACGC |
| | GAACCAAAAATGCCGCTGCC |
| | AGACCGTAGCCTTGCAGCGA |
| | GCAGCTAGGCGAGCAACTCC |
| | ATACGCGGCAGGGTACAGCG |
| | GACATCTCTGCTGCGCAGCT |
| | GTACAGCGGGGTCTTCATCT |
| | CTCGGCCATGCTGTGCAGGG |
| | CGCTGCAAGGCTACGGTCTC |
| | CCCCCACCGCTAACATCCCA |
| | GCCGTGGGATGTTAGCGGTG |
| | TGAACCAAAAATGCCGCTGC |
| | GCGTGGCCGTGGGATGTTAG |
| | GCAGCGGGGGATACGCGGCA |
| | ACGGTCTCCGGCGTGGCCGT |
| | CCGTGGGATGTTAGCGGTGG |
| | GGATACGCGGCAGGGTACAG |
| | CATCTCGGCCATGCTGTGCA |
| | GCTCCTCTGAGCCCCGCGGG |
| | TCAGTTGGCAGCTCGCCTCC |
| | TCCGGCTCCTCTGAGCCCCG |
| | AGGCGAGCAACTCCCGGCAG |
| | TGGGGGCAATGGAGGGCACC |
| | GCGCCCTCGCTGCAAGGCTA |
| | GAGCAGGAATGCGCTCTGCC |
| | GGCCGTGGGATGTTAGCGGT |
| | GGCTCCTCTGAGCCCCGCGG |
| | AGCGGCATTTTTGGTTCAGT |
| | TGAGCAGGAATGCGCTCTGC |
| | TTCAGTTGGCAGCTCGCCTC |
| | CGGCTCCTCTGAGCCCCGCG |
| | GTGCCCCCCGCGGGGCTCAG |
| | GATGTTAGCGGTGGGGGCAA |
| | GATACGCGGCAGGGTACAGC |
| | GAGGCACTCGGCGCGCCCGG |
| | TGGCCGTGGGATGTTAGCGG |
| | GTTAGCGGTGGGGGCAATGG |
| | ATGCTGTGCAGGGCGGCCAG |
| | CAGAGGAGCCGGAAGCGCCG |
| | CCTGGCCGCCCTGCACAGCA |
| | GGAAGCGCCGAGGGCGCGAG |
| | TTAGCGGTGGGGGCAATGGA |
| | AGAGGAGCCGGAAGCGCCGA |
| | CCTGCTCAGCAGCCCTCCCC |
| | CAGCCAGCGCCCTCGCTGCA |
| | TGTGCAGGGCGGCCAGGGGA |
| | GCGCTGGCTGGTGCCCCCCG |
| | GCTGGCTGGTGCCCCCCGCG |
| | GGCGCGAGCGGAGAGGCACT |
| | ACTCCCGGCAGCGGCATTTT |
| | GGCAGCGGGGGATACGCGGC |
| | GGAGAGGCACTCGGCGCGCC |
| | TGCCTCTCCGCTCGCGCCCT |
| | TACGGTCTCCGGCGTGGCCG |
| | CGCTGGCTGGTGCCCCCCGC |
| | CCGGCTCCTCTGAGCCCCGC |
| | GCTCGCGCCCTCGGCGCTTC |
| | CGCCGAGGGCGCGAGCGGAG |
| | CTTGCAGCGAGGGCGCTGGC |
| | TAGCCTTGCAGCGAGGGCGC |
| | CTGTGCAGGGCGGCCAGGGG |
| | CATGCTGTGCAGGGCGGCCA |
| | CCCGCGGGGCTCAGAGGAGC |
| | CCATGCTGTGCAGGGCGGCC |
| | CCAGGGGAGGGCTGCTGAGC |
| | TCATCTCGGCCATGCTGTGC |
| | (SEQ ID NO: 13) |
| PAX4 | TTGATGGAAGCAAAGCCCTG |
| | GCCTGGAGCATGCATCAGGA |
| | AGAGACAGGGGAAGACCTCA |
| | GAGCATGCATCAGGACGGTG |
| | AGAGTTGGCGGGTATGGGCA |
| | AGAAGGATGAGACTCCAGCT |
| | AAAAAGCTTCCCCAGAACAT |
| | GTCAGCCTGGAGCATGCATC |
| | TGTCCTGCTTCCCTTAGATC |
| | GCTGGAGTCTCATCCTTCTG |
| | CTTAGATCAGGAGAGTTGGC |
| | TGCTATTGGCCAATGTTCTG |
| | CAGAAGGATGAGACTCCAGC |
| | TCAGGAGAGTTGGCGGGTAT |
| | ATAGCAGATGAAACAGTTGA |
| | GGAGTCTCATCCTTCTGAGG |
| | CGCCAACTCTCCTGATCTAA |
| | ACCGTCCTGATGCATGCTCC |
| | GGAGCTCCTTTTCCAGCTTG |
| | CAGAGACAGGGGAAGACCTC |
| | TTCCCTTAGATCAGGAGAGT |
| | CCTTAGATCAGGAGAGTTGG |
| | CTGGGGGAAGTGGGAAGACA |
| | GTGAGGAGCCTGGGGGAAGT |
| | CCGCCAACTCTCCTGATCTA |
| | GATCTAAGGGAAGCAGGACA |
| | CAGGACGGTGAGGAGCCTGG |
| | CTGCTATTGGCCAATGTTCT |
| | AGGGAGGACAACAGAGACAG |
| | CAGGGAGGACAACAGAGACA |
| | CCAGGCTGACCCTCCTCAGA |
| | GGATGAGACTCCAGCTGGGA |
| | CTGGAAAAGGAGCTCCTAGA |
| | AGACTCCAGCTGGGAAGGCT |
| | GCCAGCCCCCAAGCTGGAAA |
| | TCTGCTATTGGCCAATGTTC |
| | GAGTCTCATCCTTCTGAGGA |
| | ATCAGGACGGTGAGGAGCCT |
| | CCTGTCTTCCCACTTCCCCC |
| | ATCAGGAGAGTTGGCGGGTA |
| | CCTTCTGAGGAGGGTCAGCC |
| | TGATCTAAGGGAAGCAGGAC |
| | TCAGGACGGTGAGGAGCCTG |
| | TCTCCTGATCTAAGGGAAGC |
| | GAGCTCCTTTTCCAGCTTGG |
| | CAGGACAGGGCAGGAAGGGA |
| | GGGGAAGTGGGAAGACAGGG |
| | CCAGCTGGGAAGGCTGGGAA |
| | TGCAGAGCCAGCCCCCAAGC |
| | GGAAGCAGGACAGGGCAGGA |
| | TCAACTGTTTCATCTGCTAT |
| | TAAGGGAAGCAGGACAGGGC |
| | ACAGGGAGGACAACAGAGAC |
| | AGGAGCTCCTTTTCCAGCTT |
| | TAGGAGCTCCTTTTCCAGCT |
| | CCCTTCCCAGCCTTCCCAGC |
| | TGGGAAGGGAAGTTCCTTCT |
| | CATCAGGACGGTGAGGAGCC |
| | GAGACTCCAGCTGGGAAGGC |
| | TCCAGCTGGGAAGGCTGGGA |
| | GAAGCAGGACAGGGCAGGAA |
| | GCAGGACAGGGCAGGAAGGG |
| | CCTGGGGGAAGTGGGAAGAC |
| | TCCTTTTCCAGCTTGGGGGC |
| | CAGGGCAGGAAGGGAGGGAC |
| | GGTGAGGAGCCTGGGGGAAG |
| | (SEQ ID NO: 14) |
| PDX1 | GCCCCGCGGAGCCTATGGTG |
| | CTGGGCCTAGCCTCTTAGTG |
| | GCCGCACCATAGGCTCCGCG |
| | TTATAGAAACATTTTCACCG |
| | GTTTGCTGCACACTCCTGAA |
| | CCGCGGAGCCTATGGTGCGG |
| | GGCCCCACGTGGTTCAGCCG |
| | GCCTGGCTGGCCGCACTAAG |
| | GGAACAAAAGCAGGTGCTCG |
| | AAAATGTTTCTATAAATGAG |
| | GAGGGAACCCACAGCCAGCG |
| | CCGCCGCACCATAGGCTCCG |
| | GCGGCCAGCCAGGCCAATCA |
| | GCAGGTGCTCGCGGGTACCT |
| | TTTCGTGAGCGCCCATTTTG |
| | GAGAAAATTGGAACAAAAGC |
| | CTGAACCACGTGGGGCCCCG |
| | AGGCTCCGCGGGGCCCCACG |
| | GCCCCCGGCTGAACCACGTG |
| | GAACAAAAGCAGGTGCTCGC |
| | AAATAGAAACTTTTAAGCCA |
| | GCTGGCCGCACTAAGAGGCT |
| | GGCCCCCGGCTGAACCACGT |
| | GCCAGGCCAATCACGGCCCC |
| | GCCTCTTAGTGCGGCCAGCC |
| | CGCCGGTCCGCGCTGGCTGT |
| | GCCCCACGTGGTTCAGCCGG |
| | CGCCGCACCATAGGCTCCGC |
| | CGGCCCCCGGCTGAACCACG |
| | CACACTCCTGAACGGGCAGC |
| | GGGCCCCACGTGGTTCAGCC |
| | GGGGCCCCACGTGGTTCAGC |
| | CTGGCGGTGCTCCCCAAAAT |
| | TTTTCGTGAGCGCCCATTTT |
| | CGGGCCGGCCGCCGCACCAT |
| | ACTCCTGAACGGGCAGCTGG |
| | GCTGGCGGTGCTCCCCAAAA |
| | AACCCACAGCCAGCGCGGAC |
| | CCACAGCCAGCGCGGACCGG |
| | TTTGCTGCACACTCCTGAAC |
| | CGCACTAAGAGGCTAGGCCC |
| | GTTCAGCCGGGGGCCGTGAT |
| | AGCAGGTGCTCGCGGGTACC |
| | GTTTTCGTGAGCGCCCATTT |
| | GCCGGGGGCCGTGATTGGCC |
| | GGGGCCGTGATTGGCCTGGC |
| | TGGTGCGGCGGCCGGCCCGC |
| | CACAGCCAGCGCGGACCGGC |
| | GTGGGGCCCCGCGGAGCCTA |
| | ACTCAGCTGAGAGAGAAAAT |
| | GCACCGCCAGCTGCCCGTTC |
| | GCCGGCCCGCCGGTCCGCGC |
| | GGAGCCTATGGTGCGGCGGC |
| | AATAGAAAATAGAAAAATAT |
| | CCGCCGGTCCGCGCTGGCTG |
| | GCCAGCGCGGACCGGCGGGC |
| | (SEQ ID NO: 15) |
| SIX2 | TGGTCCGGTTATCTGACCCG |
| | TATTATTCTAAGCGGGCATG |
| | AGTGACTGACAGCGTCTCCA |
| | GGAGCGGGGCGATCTGTCAG |
| | GTCACTGGTAACCCGAGCCT |
| | TCAGATAACCGGACCAATCA |
| | CCCCGCTCCGCGCAGAACTG |
| | GAGCCGCCCTCAGTTCTGCG |
| | CCTCAGTTCTGCGCGGAGCG |
| | GTCAGATAACCGGACCAATC |
| | GCTCCGCGCAGAACTGAGGG |
| | GGCGGCTCTACTGGAGCCTG |
| | TTCTAAGCGGGCATGAGGCG |
| | ATTTGACTCCGACTATTGTC |
| | TGCCAGCGCCAGACAATAGT |
| | GCCCTCAGTTCTGCGCGGAG |
| | ATGGAGACGCTGTCAGTCAC |
| | CTCCGACTATTGTCTGGCGC |
| | ACTGGTAACCCGAGCCTCGG |
| | ATTGGTCCGGTTATCTGACC |
| | TTAATAATATTATTCTAAGC |
| | CCCGCTCCGCGCAGAACTGA |
| | GGCCCGCGCCCTGATTGGTC |
| | TAACCGGACCAATCAGGGCG |
| | GGGCGCTCTGAGAGCCTGGG |
| | CAGGCCCCGGGTCAGATAAC |
| | CTGGTAACCCGAGCCTCGGC |
| | TGTCAGCGGAGCCGGCCGGG |
| | AAAGCTGAGAGCCAGCTAGA |
| | GGGCGATCTGTCAGCGGAGC |
| | CTTAATAATATTATTCTAAG |
| | TAAGCGGGCATGAGGCGCGG |
| | AACCGGACCAATCAGGGCGC |
| | GGGCGGCTCTACTGGAGCCT |
| | AGAACTGAGGGCGGCTCTAC |
| | TTGGTCCGGTTATCTGACCC |
| | TCTGTCAGCGGAGCCGGCCG |
| | GTCTGGCGCTGGCAGGCCCC |
| | CCGAGCCTCGGCGGGCCGGG |
| | GACTATTGTCTGGCGCTGGC |
| | CGGAGTCAAATTATTCGCCA |
| | AGAGCCTGGGAGGCGGAGAG |
| | CGTTCTCCCTCCCGTCTAGC |
| | GATCTGTCAGCGGAGCCGGC |
| | CTGTCAGCGGAGCCGGCCGG |
| | GGGAGCCGCCCGGCCCGCCG |
| | CGCCGCCGCCGGCCCCAACC |
| | TGTCTGGCGCTGGCAGGCCC |
| | AGGGCGGCTCTACTGGAGCC |
| | CCAGGCTCTCAGAGCGCCCC |
| | CCGCCCGGCCCGCCGAGGCT |
| | CGCCCGGCCCGCCGAGGCTC |
| | CCCTCAGTTCTGCGCGGAGC |
| | CTGGGGCGCTCTGAGAGCCT |
| | ATCTGTCAGCGGAGCCGGCC |
| | TAACCCGAGCCTCGGCGGGC |
| | CTGGGAGGCGGAGAGGGGCC |
| | GCGGCGGCCCGCGCCCTGAT |
| | AACCCGAGCCTCGGCGGGCC |
| | TGAGAGCCTGGGAGGCGGAG |
| | AGGCGGAGAGGGGCCGGGTT |
| | GCGGGCCGGGCGGCTCCCCC |
| | CGCTCTGAGAGCCTGGGAGG |
| | GCCGGGCGGCTCCCCCCGGC |
| | GAGAGCCTGGGAGGCGGAGA |
| | GAGGCGGAGAGGGGCCGGGT |
| | GGCGGAGAGGGGCCGGGTTG |
| | GCCGGCCGGGGGGAGCCGCC |
| | CCTGGGGCGCTCTGAGAGCC |
| | CCGGCCCCTCTCCGCCTCCC |
| | GAGAGGGGCCGGGTTGGGGC |
| | CCTGGGAGGCGGAGAGGGGC |
| | (SEQ ID NO: 16) |

### Identifying Gene Targets and Guide RNAs to Differentiate Stem Cells

### 1. Identification of Factors and Effectors

Methods and systems of the invention are utilized to identify gene targets and guide RNAs to differentiate stem cells (e.g., iPSC) into neurons, and more specifically, into dopaminergic neurons in the following example. It is understood that the methods disclosed may generally be applied to any starting cell type to produce any target phenotype and that any combination of gRNAs and effector domains to cause CRISPRa activation activity or CRISPRi inhibition activity may be employed. Preferably the transcription regulator under guidance of the dCas protein and one or more guide RNAs will cause differentiation of one of the plurality of stem cells into the viable cell or progeny thereof such that correlating the change in gene expression with the targets of the guide RNAs identifies loci to target by CRISPRa and/or CRISPRi to differentiate pluripotent stem cells into a target cell type. As the starting cells for screening, a dCas9-VPR stable iPSC cell line is created. Alternatively, any existing iPS cell line may be used.

First, NEUROD1 and NEUROG3 were identified by methods of the invention to be drivers of neural differentiation. Specifically, these gene targets were identified by bioinformatics analysis of data from a plurality of sources.

Next, using the methods and systems of the invention, the sequences (Table 4) of four (4) sgRNAs for each target gene (NEUROD1 and NEUROG3) were identified and predicted to have maximum activation in a CRISPRa system using bioinformatics analysis. The sgRNAs were then designed using methods known in the art. These synthetic sgRNAs were then transfected, either pooled or individually, into an iPSC line stably expressing the CRISPRa complex dCas9-VPR. Optionally, the dCas9-VPR complex can be introduced into iPSCs using viral vectors (e.g., lentiviral) or transposable elements (e.g., piggyBac).

TABLE 4

| Target | | |
|---|---|---|
| Gene | sgRNA ID | sgRNA Sequence |
| NEUROD1 | NEUROD1_1 | AGCAAGGCGTGGGGAGAAGT |
| | (SEQ ID NO: | |
| | 17) | |
| | NEUROD1_2 | GGGGAGCGGTTGTCGGAGGA |
| | (SEQ ID NO: | |
| | 18) | |
| | NEUROD1_3 | GCGGGAGACGAGCAAGGCGT |
| | (SEQ ID NO: | |
| | 19) | |
| | NEUROD1_4 | GTGAGGGGAGCGGTTGTCGG |
| | (SEQ ID NO: | |
| | 20) | |
| NEUROG3 | NEUROG3_1 | CACAGCTGGATTCCGGACAA |
| | (SEQ ID NO: | |
| | 21) | |
| | NEUROG3_2 | CCTCGAGAGAGCAAACAGAG |
| | (SEQ ID NO: | |
| | 22) | |
| | NEUROG3_3 | GGACAAAGGGCCGGGGTCGG |
| | (SEQ ID NO: | |
| | 23) | |
| | NEUROG3_4 | CCACACGAGGCTCTTCTCAC |
| | (SEQ ID NO: | |
| | 24) | |

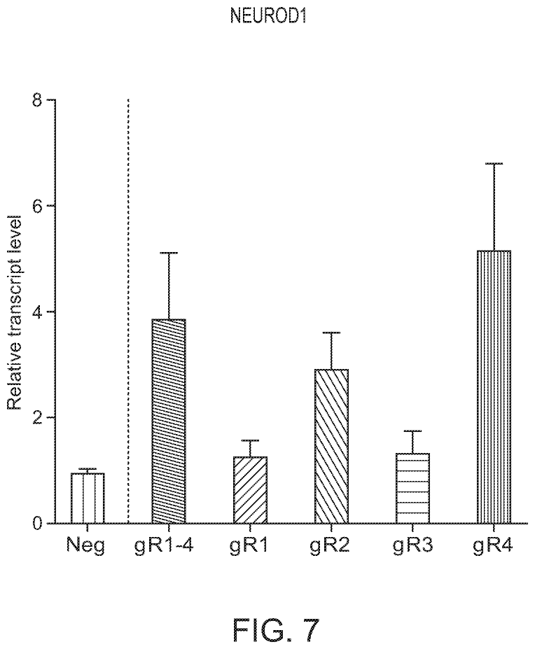

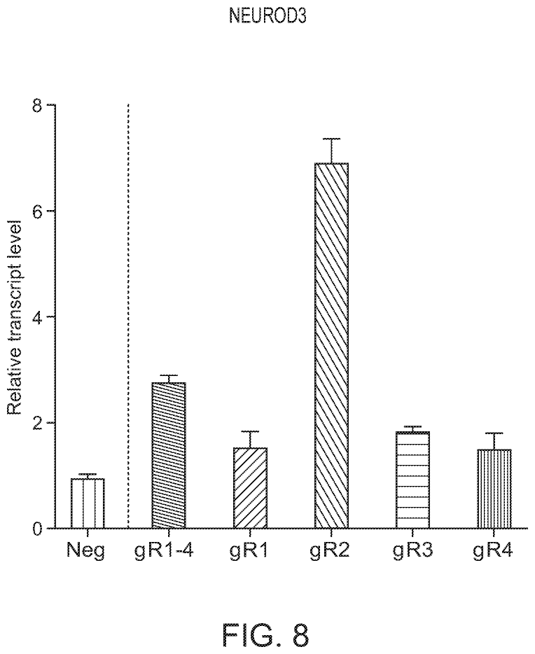

For this example, to confirm that the target genes were activated relative to a non-targeting control, qPCR gene expression analysis was performed. FIGS. 7 and 8 illustrate the RT-qPCR data that were normalized to endogenous control gene ACTB. The relative transcript levels are compared to samples transfected with a non-targeting negative control sgRNA. Error bars represent standard error of the mean (SEM) from three biologic replicates with three technical replicates each. Such analysis identified NEUROD1 sgRNA #4 and NEUROG3 sgRNA #2 as the optimal guide RNAs of each gene, respectively. The sequences of the optimal guide RNAs identified by gene expression analysis are identified as such and inputted back to the machine learning system of the invention. Optionally, the gene expression analysis does not need to be performed and pools of 4-5 sgRNAs per gene target identified by the methods of the invention can be used for cell differentiation.

### 2. Testing Identified Effectors for Differentiation Ability

Next, the identified sgRNAs were delivered to stem cells to determine their ability to direct cell differentiation towards a target cell type. Specifically, the guide RNAs (NEUROD1_4 and NEUROG3_2) delivered into the stem cells by lentiviral, express antibiotic resistance to puromycin. Other delivery methods include transient transfection (e.g., lipofection, electroporation, or NanoLaze) or stable delivery by virus (e.g., lentiviral, sendai). The induced cells are then enriched for those successfully receiving sgRNAs by FACS or drug selection.

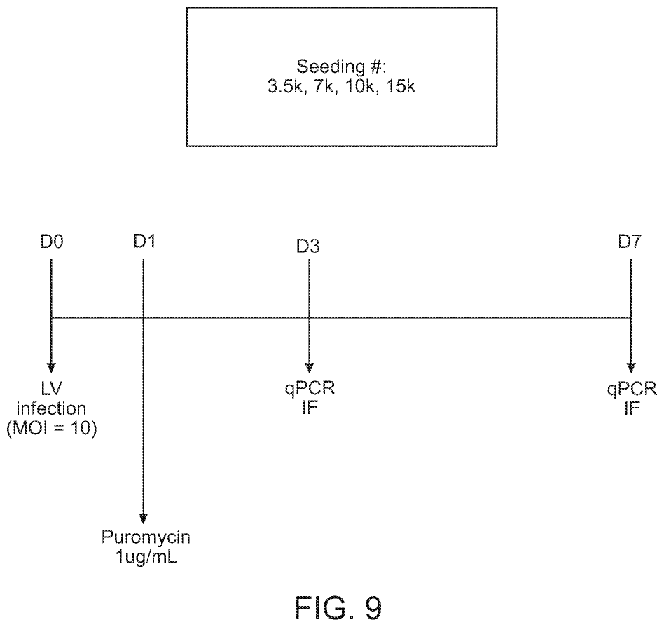

For the purpose of generating the desired neuronal cells, at day 0 dCas9-VPR iPSCs were plated at varying concentrations (3.5K, 7K, 10K, 15K) in a 96-well format and were transduced at a MOI of 10 with either each sgRNA alone, or in combination. Puromycin selection was applied at day 1 to select for cells successfully transduced with lentiviral sgRNAs. More mature cells were then collected at day 3 and at day 7 as depicted in the timeline in FIG. 9.

The stem cells are then delivered into reaction vessels (e.g., wells of a plate) such that each reaction vessel receives, on average, between zero and two of the stem cells or 10,000 to 100,000 of the stem cells, and preferably 10,000-50,000 of the stem cells. The gRNAs may have targeting portions that map to promoter regions of genes associated with a desired phenotype or trait. Each reaction vessel may receive guide RNAs that target either one or a plurality of genes associated with the desired phenotype or trait. For each gene that is targeted, between one and five distinct gRNAs may be provided. Preferably, for each gRNA that is delivered, between about one and about twenty copies of the guide RNA are delivered.

In one approach, genes are targeted individually in high throughput array format (96-well plate, 384-well plate), where activation of a single cell is desired per well. The cells may be delivered into the wells of a plate such that each well receives, on average, between 10,000 to 100,000 cells. In a single gene activation approach, individual sgRNAs per well or two to five pooled sgRNAs per gene per well are used. In another approach, all sgRNAs are pooled and delivered to a whole population of cells. When a viral vector is employed, a multiplicity of infection (MOI) is used, where each cell statistically receives either a single sgRNA or the necessary number of pooled sgRNAs. Targeted gDNA sequencing is used to confirm MOI after transduction.

In an optional workflow, when the starting cells for screening are from an existing iPS cell line, recombinant dCas9-VPR ribonucleoproteins (RNPs) complexed with barcoded sgRNA may be directly delivered to the iPSCs either in pooled or individually arrayed format. Because RNPs are transient (24-72 hours), it is necessary to perform repeat deliveries. However, this approach provides the advantage of temporal control in targeting multiple genes across a time frame (e.g., a few days to a few weeks) to determine the effects of their collective input on producing the desired target phenotype. In the pooled format, the dosage of RNPs may be titered so that each cell statistically receives more or less complex combinations of sgRNAs. In this approach, any iPS cell line can be used as the starting cell type for screening.

### 3. Characterization of Generated Cells

Cell types can be identified by cell traits characteristic of the specific cell type. Furthermore, in addition to the cell traits of a cell type, subtypes have their own cellular traits, as such, cell subtypes can be identified.

Cell traits may include cell morphology, chromosome analysis, DNA analysis, protein expression, RNA expression, enzyme activity, cell-surface markers, or a combination thereof. Characterizing the cells generated to identify their cell type can include analytic methods to assess changes in gene expression and protein expression. Such methods may include one or more of quantifying expression levels via single-cell or bulk RNA-Seq, RT-qPCR, immune staining, immune fluorescence, flow cytometry or evaluating DNA-protein interaction via chromatin immunoprecipitation and DNA sequencing (ChIP-seq).

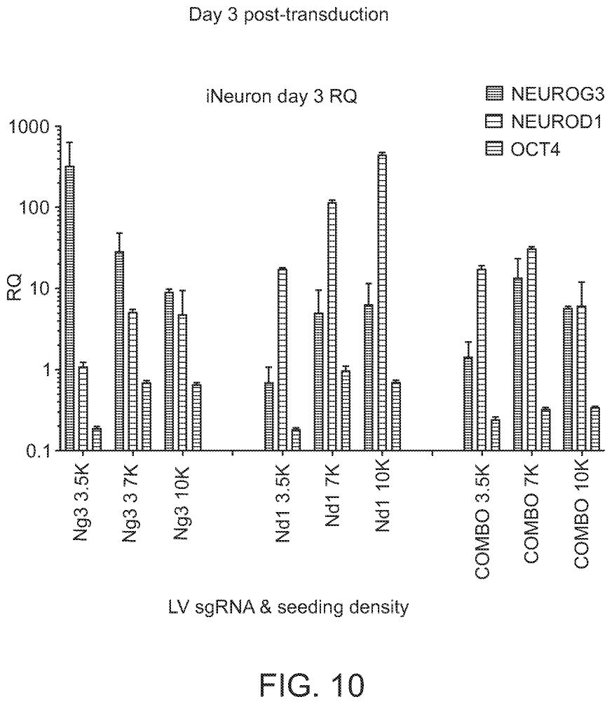

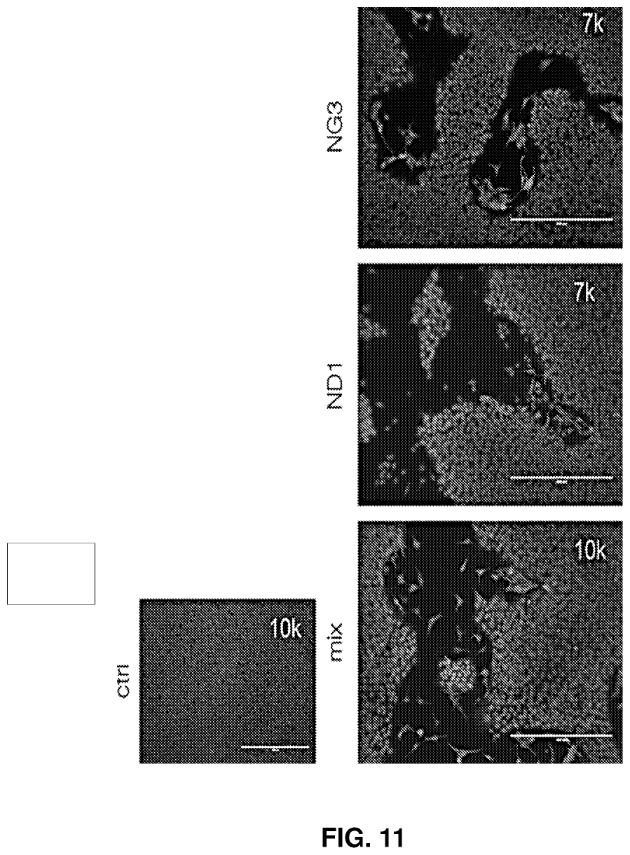

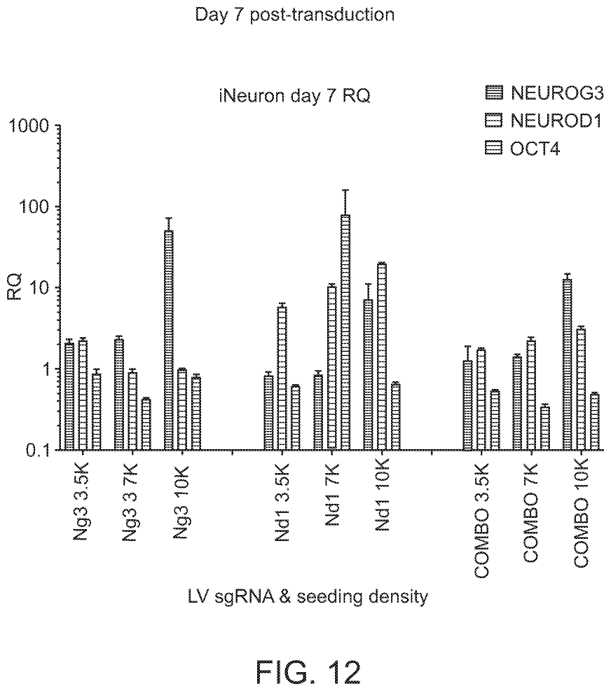

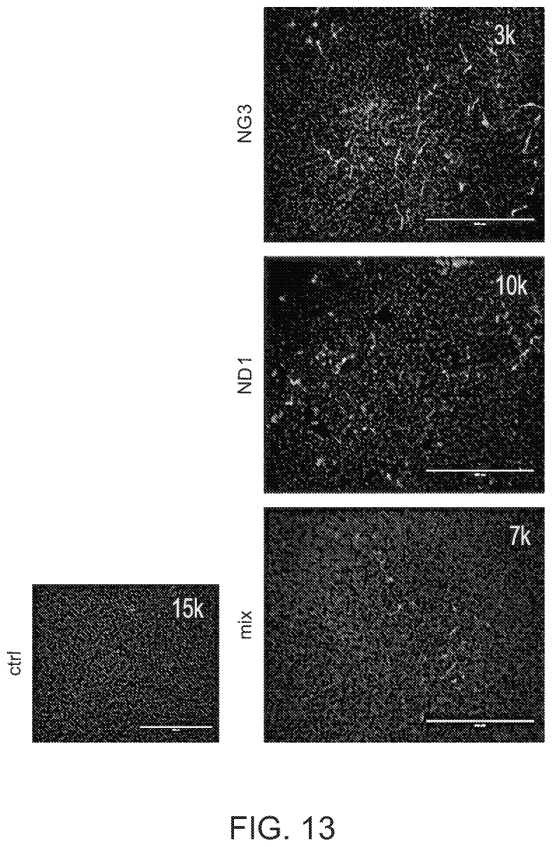

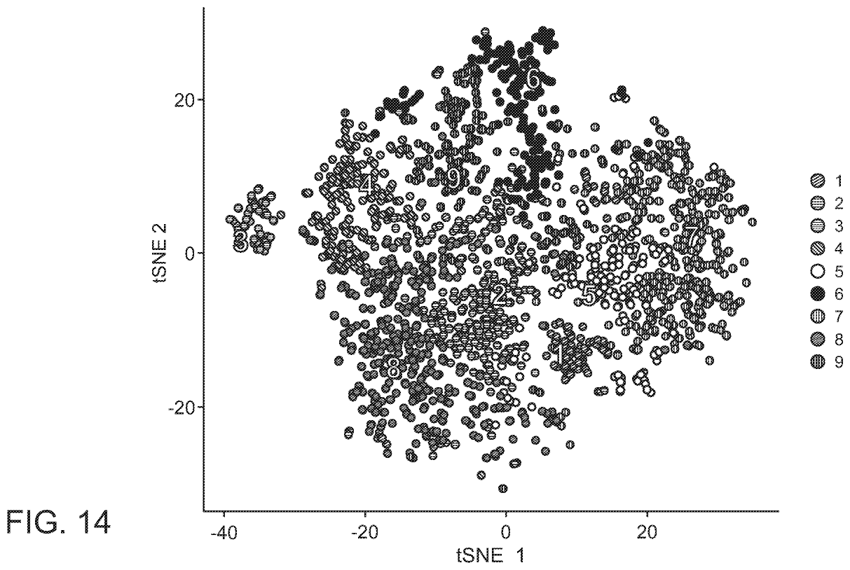

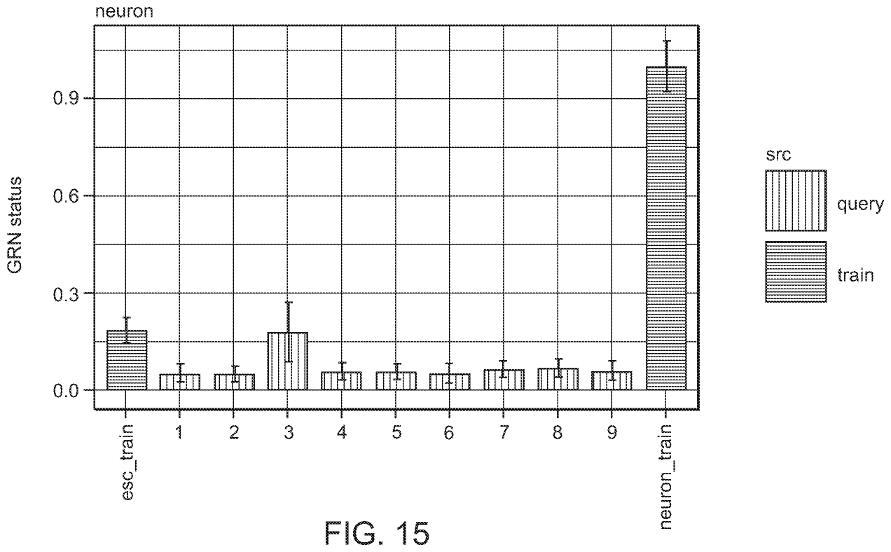

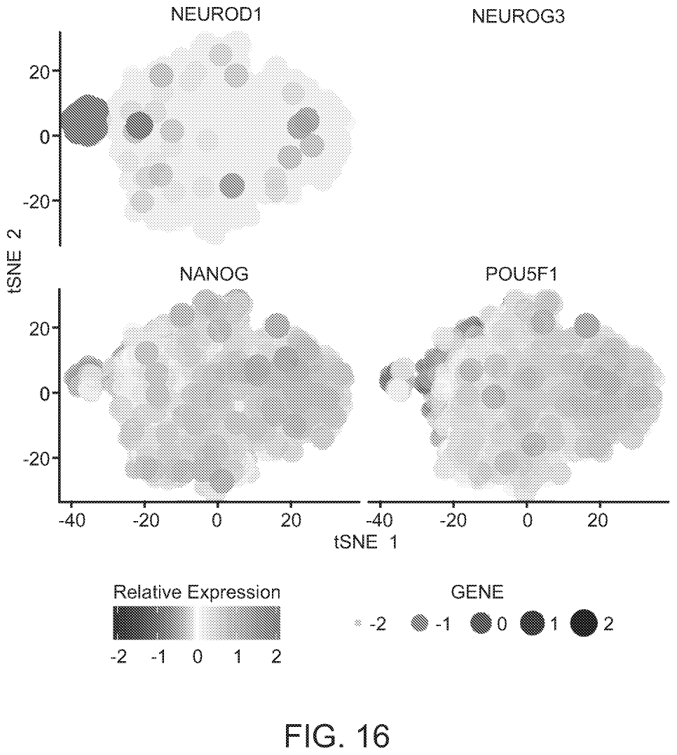

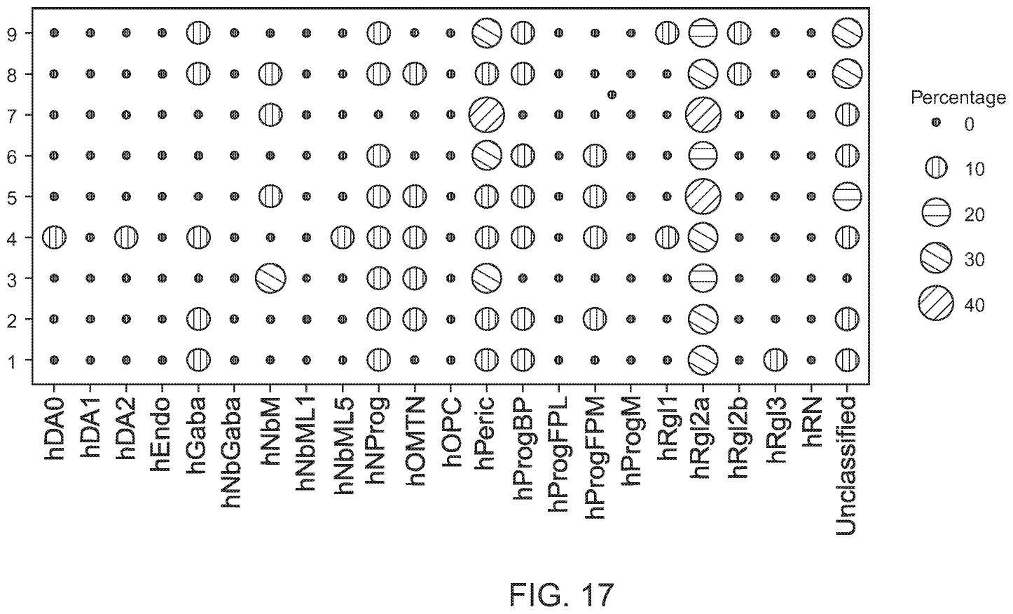

In this example, CRISPRa iPSCs transduced with lentiviral (LV) sgRNAs resulted in the formation of inducible neurons (iNeurons) by day 3. FIGS. 10 and 11 depict the changes in gene expression by staining for the neuronal-specific marker, beta III tubulin. Day 3 iNeurons possessed morphological cell traits similar to early-to-committed neuronal precursor cells, along with some mature neurons with extended neurites and arborization. As depicted in FIGS. 12 and 13, the iNeurons morphology compared to mature and varying specialized neurons with longer neurites and extensive arborization at day 7. At day 10, samples were collected for transcriptomic analysis by single-cell RNA-seq (scRNA-seq). As depicted in FIG. 14, the day 10 cells were clustered into nine (9) different groups. FIG. 15 depicts the GRN status of the different clusters using methods described herein. Furthermore, FIG. 16 provides the relative expression of the cells, of note is the saturated cluster in NEUROD1. As depicted in FIG. 17, 30% of the cells in Cluster 3 (FIG. 14) are classified as hNbM, which is a subtype of neuroblast, which is consistent with the dopaminergic neuron maturation markers disclosed. Providing that by methods of the invention, the iPSC-derived cells successfully differentiated into neuronal subtypes.

Furthermore, the kinetics of differentiation and ability of iPSCs to specify into a subtype, such as iNeuron subtypes, can be controlled by plating density, whether one or both factors are used (though either alone is sufficient for iNeuron), their relative timing, and duration of differentiation. The order in which factors are selectively turned on and off can also be used to further tune subtype specification and can be identified by methods herein. Embodiments of the disclosure include temporal control of CRISPRa/i+/−TFs. For example, in order to get to the desired synthetic dopaminergic neuron cell, CRISPRa/i may be used against a few targets for the first 2-3 days, followed by CRISPRa/i against some of the same or different genes+/−other genes expressed via PiggyBac TF for another # of days, then CRISPRa/I against some similar or other targets for the remaining # of days of differentiation. Thus, the transcription regulator under guidance of the dCas protein and one or more guide RNAs may result in differentiation of one of the plurality of stem cells when guide RNAs are introduced into at least one of the plurality of stem cells in a temporal sequence. The temporal sequence may include the introduction of a first set of one or more guide RNAs during a first period comprising one or more hours or days followed by introduction a second set of one or more guide RNAs during a second period comprising one or more hours or days. Optionally, the first set of one or more guide RNAs and the second set of one or more guide RNAs comprise wholly different guide RNAs and/or the first period and the second period do or do not overlap in time. Certain embodiments of methods of the disclosure involve using CRISPRa/i against a first set of targets during the first period, the first period comprising at least two days, and using CRISPRa/i against a second set of targets during the second period to differentiate the one of the plurality of stem cells into a dopaminergic neuron cell.

Having previously characterized the effects of NEUROD1 and NEUROG3-mediated rapid differentiation of iPSC with minimal CRISPRa targets and effectors, the identification of additional genes and guide RNAs that could mediate further cell specification to desired phenotypic subtypes was desired. As discussed, identification of the initial gene targets was performed by bioinformatics analysis of a plurality of data, as such additional gene targets and corresponding guide RNA can be identified similarly and repeated until the desired cell type or subtype is identified.

As such, in order to further differentiate the stem cells into the desired subtype of a dopaminergic neuron cell, the cycle of identifying minimal gene targets and sequences of corresponding guide RNAs was repeated.

### 4. Computational Prediction and Identification of Additional Factors

To identify additional genes responsible for differentiation of a subtype the plurality of data (including, for example publically available genomic expression data and scRNA-seq data comprised of midbrain development time courses from mouse, human, and human stem cells (Manno, 2016)) were reanalyzed. For example, a general single-cell trajectory detection algorithm, CellRouter (Lummertz da Rocha, 2018) can be used to reanalyze the data and input the results into the data for further analysis by methods of the invention. However, the outputs from CellRouter are just one data set that can be analyzed be methods of the invention.

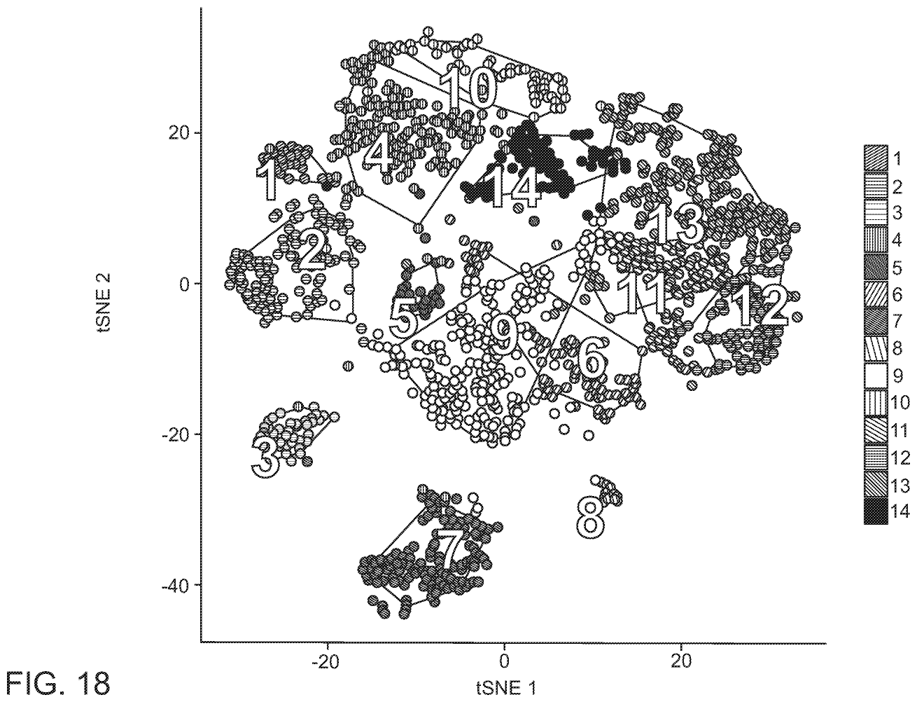

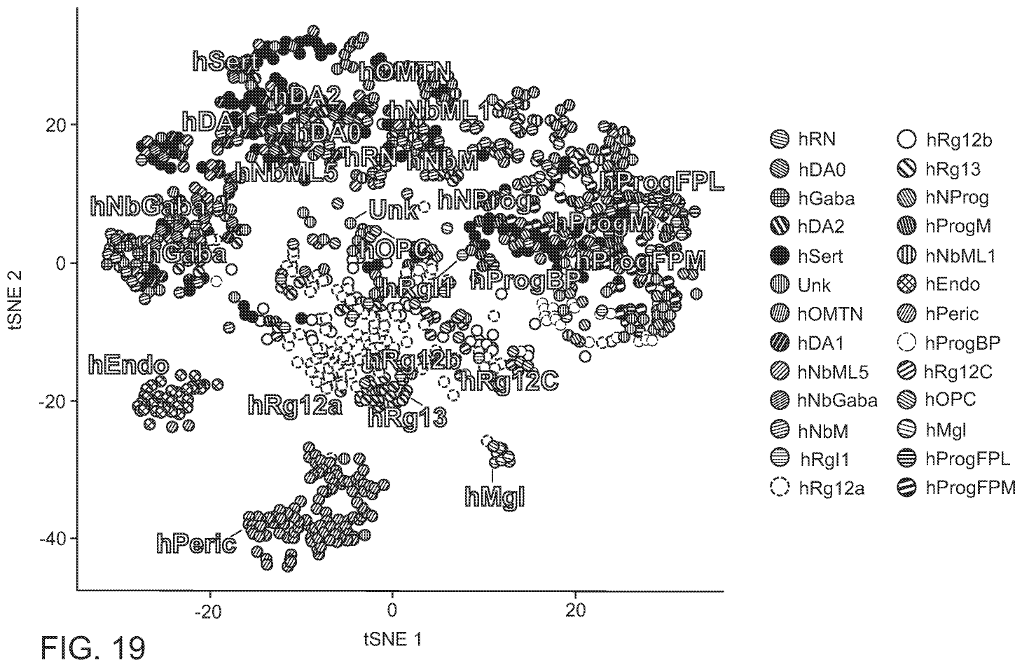

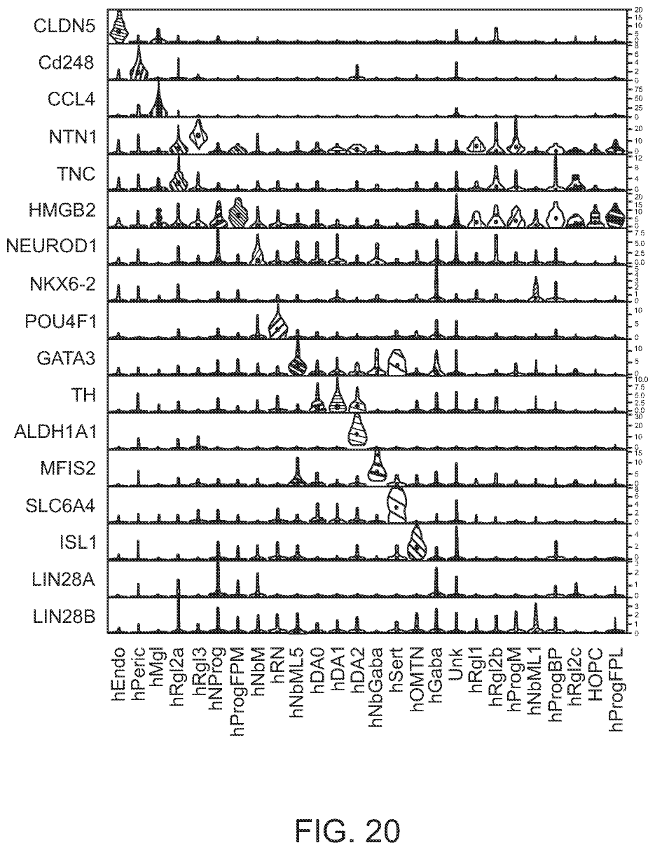

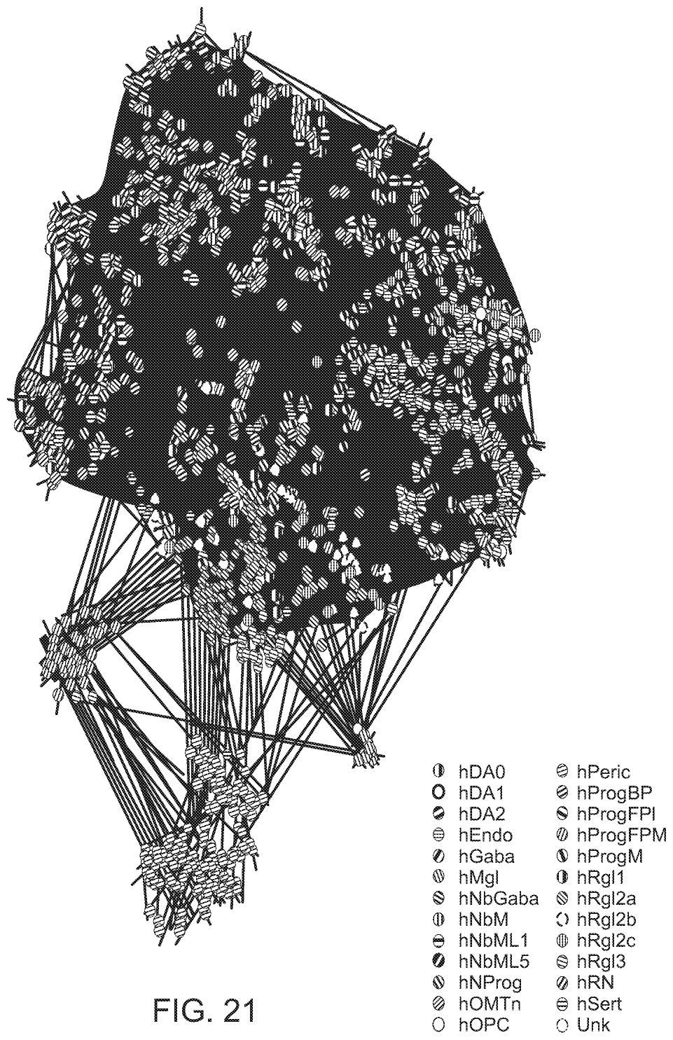

As depicted by FIG. 18, transcriptome analysis was performed on the scRNA-seq data from the developing human midbrain, and the cells were then computationally classified into distinct cell clusters by their transcriptome similarities (cell types 1-12). Furthermore, as depicted in FIG. 19, the cell clusters were then categorized into cell subtypes using previously established gene signatures for 25 cellular sub-identities. FIG. 20 depicts the gene enrichment of the different subtypes, which then allowed for the identification of the gene regulatory networks (GRNs) controlling each specific subtype's function, such as those seen in the immature neural progenitor cells (NPCs) and the more mature dopaminergic (DA) neuron subtypes. The model was then refined by reconstructing each of the identified subtype's differentiation trajectories, as depicted in FIG. 21.

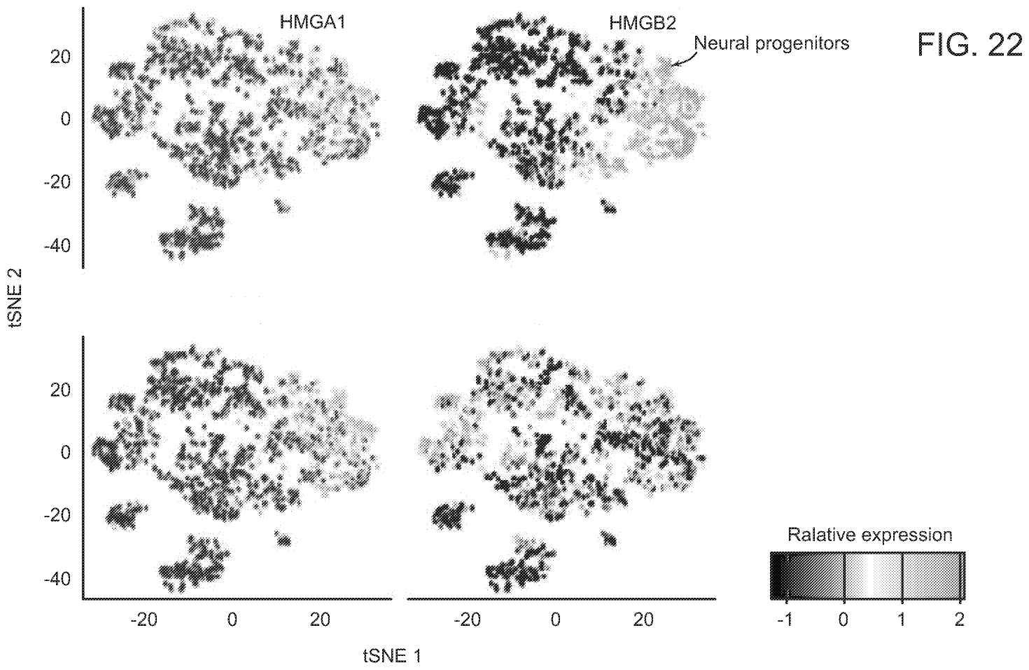

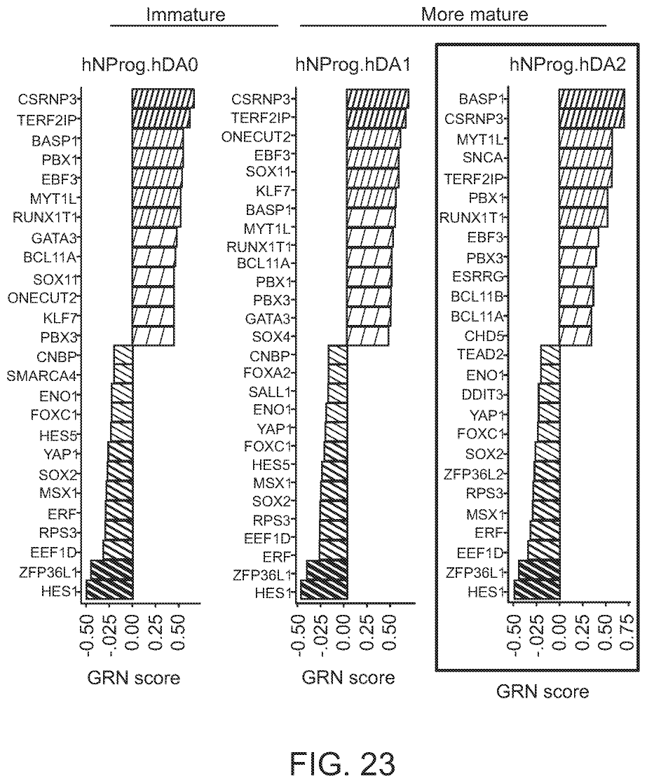

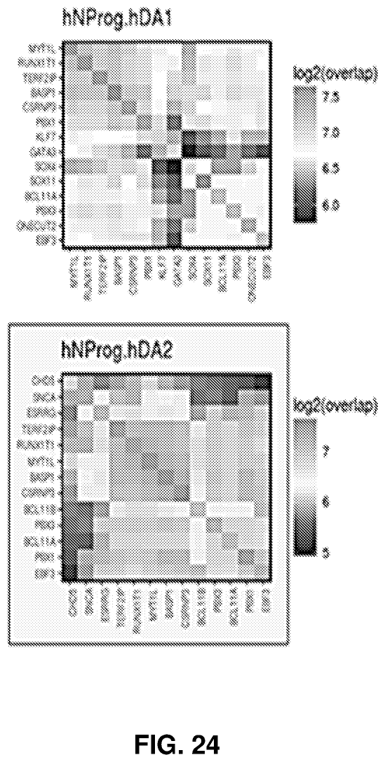

For example, FIG. 22 depicts four (4) t-SNE plots mapping genes (HMGA1, HMGB2, OTX2 and PBX1) previously known to be involved in differentiation from NPCs to dopaminergic neurons and the different subtypes of cells to show the gene expression of those genes in those types of cells. Such a detailed reconstruction of the differentiation pathways from the immature NPCs to both immature (hDA0) and more mature (hDA1 and hDA2) dopaminergic neurons was established by this analysis. The genes associated with the differentiation pathways were then assigned a GRN score using the methods described herein, thereby identifying the top 13 up-regulated and top 13 down-regulated genes responsible for establishing the GRNs responsible for each cellular subtype identity (FIG. 23). These top-level nodes (or genes) were mapped onto downstream networks (FIG. 24), by prioritizing minimal overlap of downstream targets and maximum GRN score, the identification of a minimal set of two genes (BASP1 and SNCA) were predicted to differentiate NPCs into mature dopaminergic neurons (hNProg.hDA2) (FIG. 24).

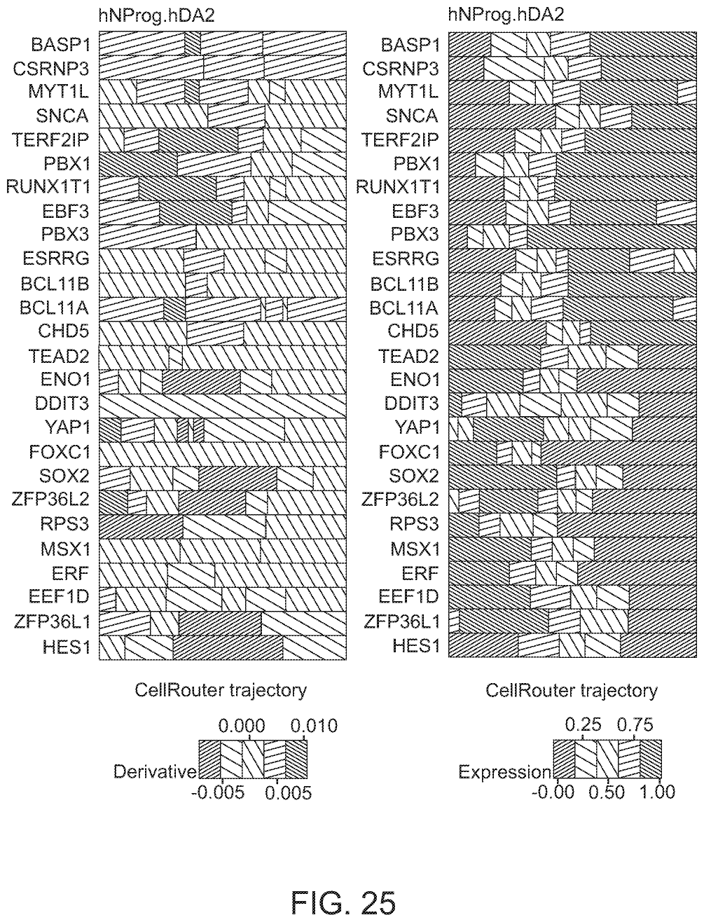

The temporal sequence of gene activation necessary to establish DA neuron identity was then established. Through the trajectory reconstruction analysis, a temporal timing of gene activation based on the identified relationship-based lineage trajectory mapping of the scRNA-seq data set with step-wise progressions through development was established. As shown in FIG. 25, the relative expression levels of the top-level nodes for mature dopaminergic neurons were plotted across time (right), and the derivate of these expression levels (left) identifies inflection points in gene expression. For example, it is identified from these data that to achieve a DA neuron fate from NPCs, over-expression of BASP1, then SNCA is necessary. Based on the system's analysis of the dynamics of GRNs during hDA2 differentiation from NPCs previously identified, MYT1L and BASP3 are predicted to regulate each other. Therefore, overexpression of BASP1 is predicted to induce expression of MYT1L.

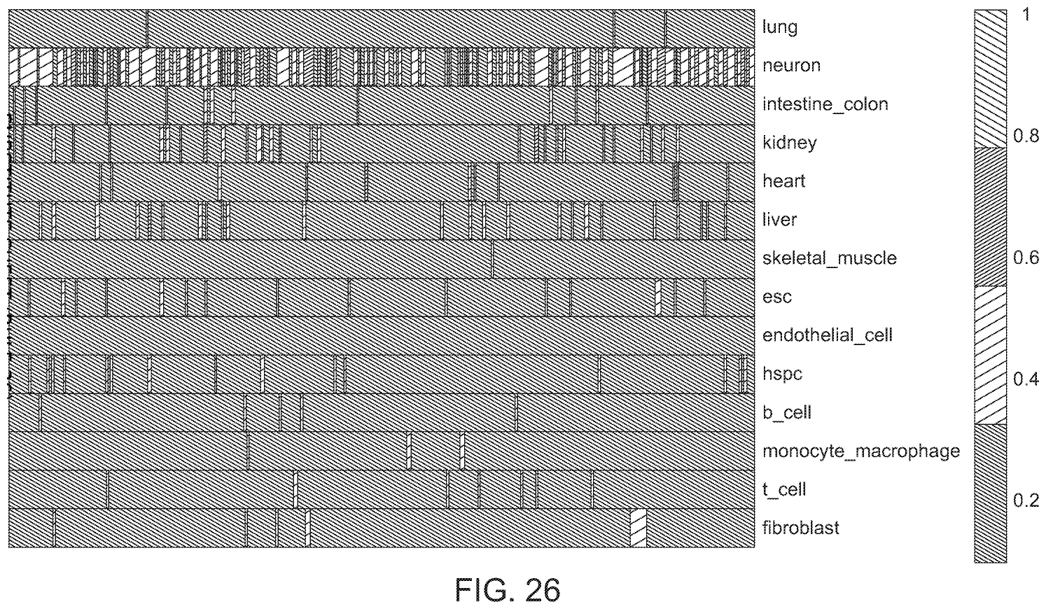

The predicted manipulation of GRNs and resulting cells were computationally verified using the CellNet (Cahan, 2014) system, the results of which are represented in FIGS. 26-28. The dataset was processed profiling a time-course (42 e 63 hours) of neuron differentiation of iPSCs to identify whether iPSC-derived neurons are transcriptionally similar to neurons present in the CellNet training dataset. The heat map of FIG. 26 depicts the probability of iPSC-derived neurons relative to neurons in the training dataset (classification scores). For example, values close to 1 indicate that there is a high probability of iPSC-derived neurons to be molecularly similar to neurons in the CellNet training dataset, which represents a broad neuron type.

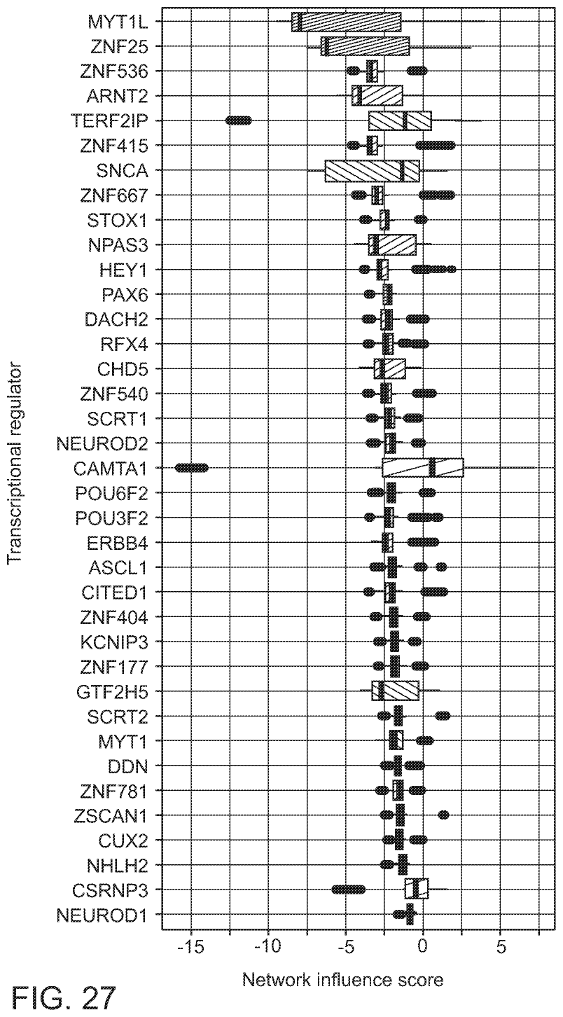

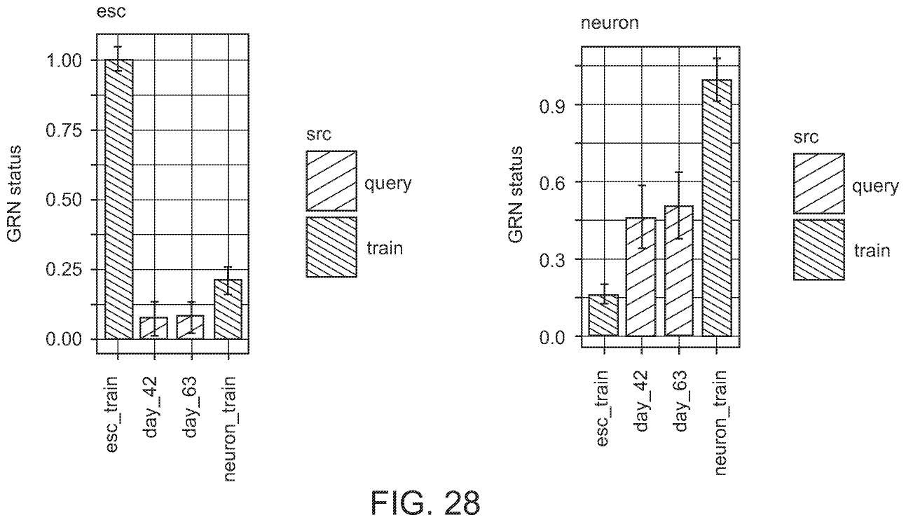

Moreover, reconstruction of the cell-type specific GRNs that represent the ‘identity’ of a particular cell type can be outputted by CellNet. The GRN status quantifies the establishment of a cell-type specific GRN in the query samples, providing enhanced resolution to determine the activation or repression of a source (e.g., iPSC) and target cell type (e.g., neuron) GRN. FIG. 27 depicts the ‘embryonic stem cell’ (ESC) GRN as silenced during iPSC differentiation towards the neuronal fate (left plot), while the neuron GRN is activated, consistent with the induction of the neuronal fate. Finally, the Network Influence Score (NIS), presented in FIG. 28, aim at identifying transcriptional regulators to enhance cell engineering by quantifying the extent of dysregulation of these transcriptional regulators relative to the training dataset. This analysis provides that iPSC-derived neurons lack expression of critical neuron genes such as MYT1L and SNCA. This analysis verified that the predicted genes and temporal sequence of expression would generally produce neurons. Unfortunately, the CellNet system is unable to map to individual neural sub-identities.

However, the presently disclosed methods and systems, are able to receive this data (e.g., GRNs, genes, temporal expression of the genes as outputted by the CellRouter system) and continuously train the data set to classify cellular subtypes and verify resulting cell types, subtypes and phenotypes computationally, which is a vast improvement over the prior art. As described throughout, the system receives data from many other data sources and internally generated scRNA-seq data. Upon verification of the subtype in silico, the sequences of a minimum number of guide RNAs for each of the identified additional gene targets are identified using the methods and systems described herein.

### 5. Iteration Until the Desired Cellular Phenotype is Achieved

Using the verified additional gene predictions from the system and their corresponding guide RNAs, dCas9-VPR iPSCs were transduced using the methods described herein with lentiviral sgRNAs activating NEUROD1, BASP1, and SNCA in different permutations and temporal combinations as identified by methods of the invention. The result of which was the specification of dopaminergic neurons from stem cells.

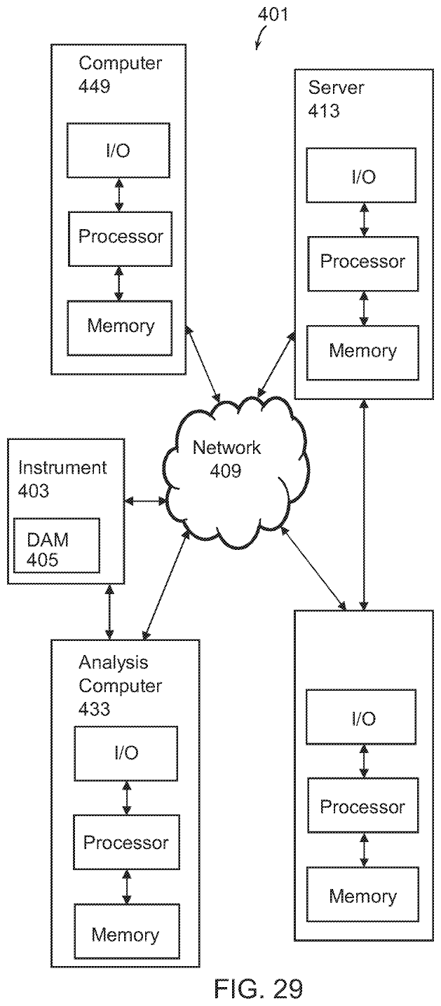

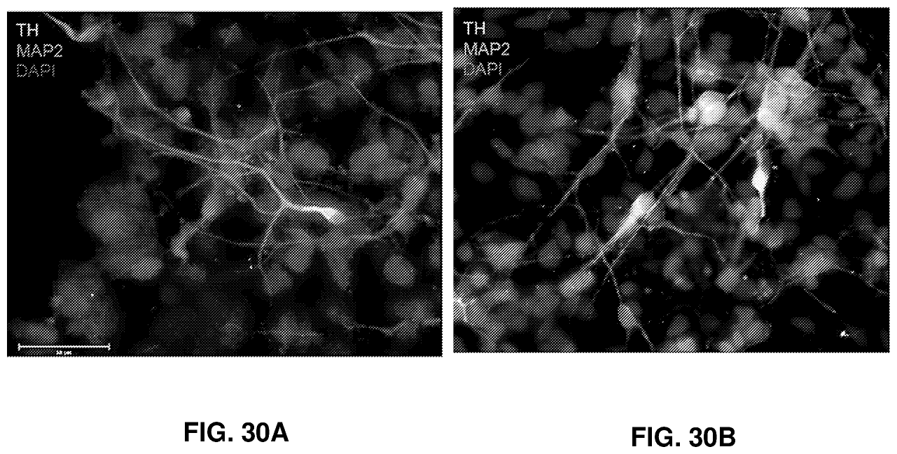

FIGS. 30A-30B depict the change in expression of TH, MAP2 and DAPI between the intermediate neurons above and the dopaminergic neurons resulting from this experiment, respectively. As depicted there is an overall increase of expression in the dopaminergic neurons (FIG. 29B) and a particular increase in MAP2 and TH.

However, if the desired phenotype is not achieved after a single process through the disclosed system and methods, then the “design-build-test” cycles (i.e., steps 2-4 of this example) are repeated until the desired cellular type is identified using the disclosed systems and methods.

For example, to achieve mature dopaminergic neurons with the phenotype of functionally secreting dopamine, another cycle through steps 2-4 was performed to identify and verify additional genes and their corresponding guide RNAs. Particularly, the system identified the particular permutation and temporal combination of first round (day 0-day 15) with a single sgRNA of NEUROD1_4 targeting NEUROD1 and second round with two different sets of sgRNAs targeting MYTL1 (day 15-day 35) and ESRRG (day 18-day 35), respectively.

On day 0, CRISPRa iPSCs were transduced with lentiviral (LV) NEUROD1_4 sgRNAs targeting NEUROD1 and incubated for 24 hours, after which the cells were rinsed with PBS and medium was changed into a medium to support the growth of stem cells (e.g., StemFlex). At day 3, the medium was changed to a nutrient rich medium (e.g., NB medium) with puromycin for the first 3-5 days and changed every 2nd day (days 5, 7, 9, 11). On day 13, the cells were dissociated with recombinant cell-dissociation enzymes and re-plated. Half of the medium was changed every 2 days thereafter.

At day 15 lentiviral sgRNAs identified by the system targeting MYTL1 were added (i.e., protospacer pool GAACAGAAGGUCAUAUGCCG (SEQ ID NO: 25), GGAUAGGCUCGCAGGCCUCA (SEQ ID NO: 25), GGCCUCAUAGAUAAUGAUGA (SEQ ID NO: 27) and GGAGUGGGAGCGUGUGCAUG (SEQ ID NO: 28)). At day 18 lentiviral sgRNAs identified by the system targeting ESRRG (i.e., protospacer pool GAGGCUGCCAGGUUCUCCUC (SEQ ID NO: 29), GUAACCACCCGAGGAGAACC (SEQ ID NO: 30), GUUCUCCUCGGGUGGUUACG (SEQ ID NO: 31) and GGUCGCGGGAGCCCAGUUAA (SEQ ID NO: 32)) were added. Table 5 provides the gRNA sequences.

TABLE 5

| Target | | |
|---|---|---|
| Gene | sgRNA ID | sgRNA Sequence |
| MYTL1 | MYTL_1 | GAACAGAAGGTCATATGCCG |
| | (SEQ ID NO: 33) | |
| | MYTL_2 | GGATAGGCTCGCAGGCCTCA |
| | (SEQ ID NO: 34) | |
| | MYTL_3 | GGCCTCATAGATAATGATGA |
| | (SEQ ID NO: 35) | |
| | MYTL_4 | GGAGTGGGAGCGTGTGCATG |
| | (SEQ ID NO: 36) | |
| ESRRG | ESRRG_1 | GAGGCTGCCAGGTTCTCCTC |
| | (SEQ ID NO: 37) | |
| | ESSRG_2 | GTAACCACCCGAGGAGAACC |
| | (SEQ ID NO: 38) | |
| | ESSRG_3 | GTTCTCCTCGGGTGGTTACG |
| | (SEQ ID NO: 39) | |
| | ESSRG_4 | GGTCGCGGGAGCCCAGTTAA |
| | (SEQ ID NO: 40) | |

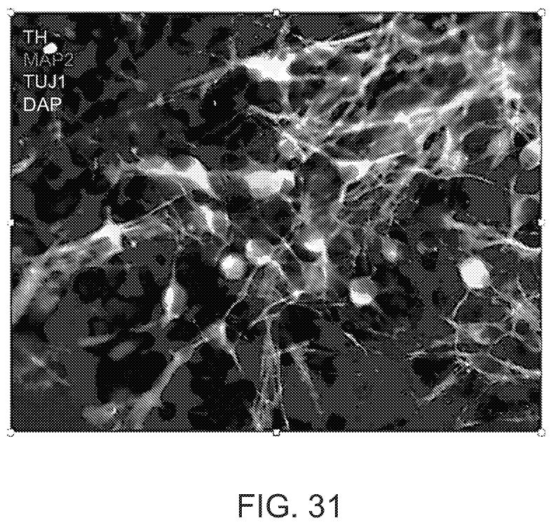

FIG. 31 depicts the change in expression of TH, MAP2 and DAPI and the additional expression of TUJ1 of the dopamine neurons after 35 days of cell differentiation. At day 35, the mature dopaminergic neurons' dopamine secretion was measured by colorimetry of the cell lysates following protocols known in the art (e.g., Universal Dopamine Elisa kit) and using commercially available control neuroblastoma cell lines (e.g., SH-Sy5y neuroblastoma line differentiated for 35 days according to published, SH-Sy5y specific protocol).

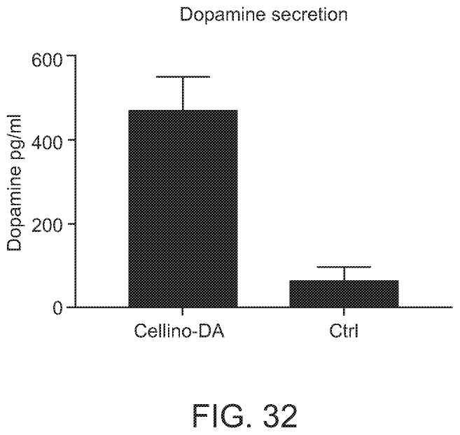

As provided in FIG. 32, the mature functionally-secreting dopamine cells produced by the methods described herein secrete significantly more dopamine than the control cells. As such, the methods and systems of the invention described herein identify targets and corresponding guide RNAs that provide targeted cell differentiation, and achieve superior phenotypic results.

As contemplated throughout, one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus. FIG. 29 diagrams the system 401 for identifying the minimum number of targets to specify cell fate. The system 401 includes at least one computer 449, such as a laptop or desktop computer, than can be accessed by a user to initiate methods of the invention and obtain results. The system 401 preferably also includes at least one server sub-system 413 and either or both of the computer 449 and the server sub-system 413 may include and provide the machine learning system of the invention. The server subsystem 413 may have a dedicated terminal computer 467 for accessing the server sub-system 413. Additionally, in some embodiments, the system 401 operates in communication with a laboratory, which may include an analysis instrument 403 such as a gene expression instrument. The analysis instrument 403 may have its own data acquisition module 405, such as, for example, the electronic instruments of a single-cell RNA sequencer, an RNA multiplex sequencer (e.g., nCounter) , a microarray, or RT-qPCR. The instrument 403 may have its own built-in or connected instrument computer 433. Any or all of the computer 449, server subsystem 413, terminal computer 467, instrument 403, and instrument computer 433 may exchange data over communications network 409, which may include elements of a local area network (LAN), a wide area network (WAN) the Internet, or combinations thereof. Each of computer 449, server subsystem 413, terminal computer 467, and instrument computer 433, when included, preferably includes at least one processor coupled to one or more input/output devices and a tangible, non-transitory memory subsystem. The I/O devices may include one or more of: monitor, keyboard, mouse, trackpad, touchpad, touchscreen, Wi-Fi card, cellular antenna, network interface cards, or others. The memory subsystem preferably includes one or more of RAM and a disc drive, such as a magnetic hard drive or solid state drive.

Memory according to the invention can include a machine-readable medium on which is stored one or more sets of instructions (e.g., software) embodying any one or more of the methodologies, functions or outputs of the methodologies described herein. The software may also reside, completely or at least partially, within the main memory and/or within the processor during execution thereof by the computer system, the main memory and the processor also constituting machine-readable media. The outputs include programs for the temporal expression of the identified gene targets to achieve cell fate specification. Optionally, these programs also include guide RNA sequences respective to the gene targets. The software may further be transmitted or received over a network via the network interface device. Ultimately, the systems disclosed herein, encompasses a generalizable iterative system capable of identifying minimal target genes and their minimal effectors (i.e., guide RNAs) capable of directing cell differentiation between any given cell identities (e.g. iPSCs, NPCs, and DA neurons), ultimately directing cell fate of a cell through machine learning and experimental validation.

Furthermore, sets of genes, or gene modules, can be identified as being responsible for affecting a certain phenotype of cell. The gene modules identified can be utilized in any cell type to effectuate the desired phenotype. In contrast to the prior art, the methods disclosed enable the generalizable selection of a minimal number of targets with a corresponding minimum number of gRNAs that direct cell fate specification with additional maturation through identification of additional targets via machine learning methods in iterative design-build-test cycles.

What is claimed is:

1. A screening method for identifying targets involved in cell differentiation, the method comprising:
introducing into each of a plurality of stem cells a dCas protein linked to a transcription regulator and one or more guide RNAs;
isolating, from the plurality of stem cells, a viable cell that contains the dCas protein linked to the transcription regulator and at least one of the guide RNAs;
measuring gene expression in the viable cell or progeny thereof; and
correlating a change in gene expression in the viable cell or progeny thereof with one or more targets of the guide RNAs in the viable cell.

2. The method of claim 1, wherein the transcription regulator under guidance of the dCas protein and one or more guide RNAs results in differentiation of one of the plurality of stem cells into the viable cell or progeny thereof such that correlating the change in gene expression with the targets of the guide RNAs identifies loci to target by CRISPRa and/or CRISPRi to differentiate pluripotent stem cells into a target cell type.

3. The method of claim 2, further comprising initiating expression of, or introducing, one or more additional gene products to promote differentiation of the one of the plurality of stem cells into the viable cell or progeny thereof.

4. The method of claim 3, wherein expression of at least one of the additional gene products is initiated by one selected from the group consisting of: introducing a corresponding gene using a PiggyBac transposon; introducing a corresponding gene via a plasmid or viral vector;
introducing an mRNA encoding the gene product.

5. The method of claim 3, wherein at least one of the additional gene products is introduced as a protein to the one of the plurality of stem cells.

6. The method of claim 3, wherein the gene product is a transcription factor and the transcription factor and the transcription regulator under guidance of the dCas protein and one or more guide RNAs results in differentiation of the one of the plurality of stem cells into a beta islet cell.

7. The method of claim 2, wherein guide RNAs are introduced into at least one of the plurality of stem cells in a temporal sequence.

8. The method of claim 7, wherein the temporal sequence includes the introduction of a first set of one or more guide RNAs during a first period comprising one or more hours or days followed by introduction a second set of one or more guide RNAs during a second period comprising one or more hours or days.

9. The method of claim 8, wherein the first set of one or more guide RNAs and the second set of one or more guide RNAs comprise wholly different guide RNAs and/or the first period and the second period do or do not overlap in time.

10. The method of claim 8, further comprising using CRISPRan against a first set of targets during the first period, the first period comprising at least two days, and using CRISPRan against a second set of targets during the second period to differentiate the one of the plurality of stem cells into a glucose-responsive insulin-secreting beta cell.

11. The method of claim 1, wherein isolating the viable cell includes selecting a cell that exhibits a desired trait.

12. The method of claim 11, wherein selecting the cell that exhibits the desired trait includes staining the plurality of stem cells with a marker for the desired trait, and sorting the cells on a fluorescence-activated cell sorting instrument.

13. The method of claim 12, wherein the desired trait includes a specified differentiated cell type and the marker includes a protein expressed by the differentiated cell type.

14. The method of claim 13, wherein the desired trait includes a beta cell phenotype, and marker one or more of the presence of C-peptide, Insulin, Chromogranin A, and Nkx6.1, and the absence of Glucagon and Somatostatin.

15. The method of claim 1, wherein measuring gene expression in the viable cell or progeny thereof includes one or more of: quantifying expression levels via RNA-Seq; and evaluating DNA-protein interaction via chromatin immunoprecipitation and DNA sequencing (ChIP-seq).

16. The method of claim 15, further comprising determining fold-change in expression level of a transcript associated with the marker by normalizing read counts from the measuring against control read counts.

17. The method of claim 16, wherein the guide RNAs are barcoded, and the method further comprises using a computer system to analyze sequence data to determine the fold-change for the transcript and correlate, using barcode sequences in the sequence data, the fold-change for the transcript with the one or more targets of the guide RNAs in the viable stem cell.

18. The method of claim 1, wherein introducing the dCas protein linked to the transcription regulator into the stem cells includes delivering to the stem cells a vector that encodes a fusion protein comprising the dCas protein and the transcription regulator.

19. The method of claim 18, wherein the vector comprises a viral vector, a plasmid, or transposable element.

20. The method of claim 19, wherein the vector further comprises a selection marker, and the method further comprises selecting for cells transformed by the vector prior to the isolating step.

21. The method of claim 20, wherein the cells are selected for transformation by the vector prior to introducing the one or more guide RNAs.

22. The method of claim 1, further comprising distributing the plurality of stem cells into reaction vessels such that each reaction vessel receives, on average, between 0 and 2 of the stem cells.

23. The method of claim 22, wherein introducing the one or more guide RNAs includes obtaining guide RNAs that have targeting portions that map to promoter regions of genes associated with a desired phenotype or trait, and delivering to each reaction vessel guide RNAs that target either one or a plurality of genes associated with the desired phenotype or trait.

24. The method of claim 23, wherein for each gene that is targeted, between one and 40 distinct guide RNAs are delivered.

25. The method of claim 23, wherein for each guide RNA that is delivered, between about 1 and about 20 copies of the guide RNA are delivered.

26. The method of claim 1, wherein isolating the viable stem cell includes selecting a cell that exhibits a specified differentiated cell type, wherein the guide RNAs have targeting portions that map to promoter regions of genes associated with the differentiated cell type, and the method further includes promoter regions of genes to target for transcription regulation using a dCas protein linked to a transcription regulator to differentiate stem cells to the specified differentiated cell type.

27. The method of claim 1, further comprising identifying the one or more targets of the guide RNAs by:
sequencing at least a portion of the guide RNAs to produce sequence reads; and
mapping the sequence reads to a reference to identify genomic loci targeted by the guide RNAs.

28. The method of claim 27, wherein the viable cell or progeny thereof are differentiated cells of a specific cell type.

29. The method of claim 28, further comprising identifying the differentiated cells by sequencing nucleic acid from the differentiated cells.

30. The method of claim 29, wherein the nucleic acid comprises gene transcripts resulting from transcriptional activation by the dCas protein linked to the transcription regulator.

31. The method of claim 30, wherein the guide RNAs and gene transcripts are sequenced via RNA-Seq using a next-generation sequencing platform.

32. The method of claim 1, further comprising determining a network of targets involved in directing cell differentiation by identifying a plurality of targets involved in directing the stem cells to a target phenotype.

33. The method of claim 1, wherein the stem cells comprise induced pluripotent stem cells.

34. The method of claim 1, wherein the transcription regulator comprises one or more effector domains that recruit coactivator or corepressor proteins to the dCas protein-linked transcription regulator.

35. The method of claim 1, wherein the introducing the dCas proteins and delivering the guide RNAs are done as a single step by providing the stem cell with a ribonucleoprotein (RNP) comprising the dCas protein linked to the transcription regulator and complexed with one of the guide RNAs.

36. The method of claim 1, wherein introducing the dCas proteins and delivering the guide RNAs includes providing the each of the stem cells with: an mRNA encoding a fusion protein that includes the dCas protein and the transcription regulator; and at least one of the guide RNAs.

37. The method of claim 1, wherein introducing the dCas proteins includes delivering a vector comprising a gene for a fusion protein that includes the dCas protein and the transcription regulator.

38. The method of claim 37, wherein the transcription regulator comprises a domain that recruits coactivator or corepressor proteins to the fusion protein.

39. A method for identifying targets involved in cell differentiation, the method comprising:
introducing into each of a plurality of stem cells a complex comprising a dCas protein linked to an effector domain and complexed with a guide RNA; and
identifying a genomic loci targeted by a guide RNA introduced to one of the stem cells that differentiated into a desired cell type, thereby identifying transcription regulation targets for directing cell differentiation to the desired cell type.

40. The method of claim 39, wherein introducing the complexes includes:
delivering to each of the stem cells a vector that encodes a fusion protein comprising the dCas protein linked to the effector domain and a guide RNA.

41. The method of claim 39, wherein sequences of the guide RNAs are known before the introducing and the method further comprises selecting a cell of the desired cell type and associating the selected cell with the sequence of the corresponding guide RNA.

42. The method of claim 39, wherein the identifying step comprises selecting a cell of the desired cell type and performing an assay to determine a sequence of the guide RNA introduced into that cell, wherein the assay comprises next-generation sequencing.

43. The method of claim 39, wherein the effector domains are domains that recruit coactivator or corepressor proteins to the complex.

44. The method of claim 39, wherein the identified genomic loci are within a human genome.

45. A method for directing cell fate, the method comprising:
determining a minimum number of genes required for differentiation of a stem cell into a selected cell type;
exposing said stem cell to a Cas endonuclease and associated guide RNAs directed at a portion of said genes; and
identifying members of said selected cell type and isolating said members.

46. The method of claim 45, wherein the genes and the guide RNAs are identified by analyzing data obtained from a plurality of sources.

47. The method of claim 46, further comprising:
adding the genes and the guide RNAs into the data; and
continuing to identify genes involved in cell differentiation of the cell type and guide RNAs directed at a portion of said genes in the data that includes the genes and the guide RNAs initially introduced.

48. The method of claim 45, further comprising repeating the method until the cell type is identified in at least one cell.

49. The method of claim 45, wherein the cell type has specific cell traits.

50. The method of claim 49, wherein cell traits comprise morphology, chromosome analysis, DNA analysis, protein expression, RNA expression, enzyme activity, or cell-surface markers, or a combination thereof.

51. The method of claim 50, wherein the members are identified by comparing cell traits of the members to the specific cell traits of the cell type.

52. The method of claim 45, wherein the guide RNAs are introduced into the stem cells in a temporal sequence.

53. The method of claim 52, wherein the temporal sequence includes the introduction of a first set of one or more of the guide RNAs during a first period comprising one or more hours or days followed by introduction a second set of one or more of the guide RNAs during a second period comprising one or more hours or days.

54. The method of claim 53, wherein the first set of guide RNAs and the second set of guide RNAs comprise wholly different guide RNAs and/or the first period and the second period do or do not overlap in time.

55. The method of claim 53, further comprising using CRISPRa/i against a first set of targets during the first period, the first period comprising at least two days, and using CRISPRa/i against a second set of targets during the second period to differentiate the one of the plurality of cells into a dopaminergic neuron.

56. A method for identifying a minimal number of targets and minimum number of guide RNAs to direct cell fate specification, the method comprising:
analyzing data to identify a plurality of targets involved in cell differentiation of a desired cell type;
selecting a minimal number of the targets;
analyzing the data to identify sequences of guide RNAs for the targets, wherein the sequences map to promoter regions of the targets associated with a phenotype of the desired cell type, and wherein the guide RNAs are a minimum number of guide RNAs for the targets to promote cell differentiation to the desired cell type.

57. The method of claim 56, further comprising analyzing the data to identify additional targets involved in cell fate specification of a subtype of the desired cell type, selecting a minimum number of the additional targets, and analyzing the data to identify sequences of additional guide RNAs for each of the additional targets, wherein the sequences map to promoter regions of the additional targets associated with a phenotype of the desired cell subtype, and wherein the additional guide RNAs are a minimum number of additional guide RNAs for each of the additional targets to further promote cell differentiation to the desired cell subtype.

58. A machine learning system for identifying targets and guide RNAs to direct cell fate, the system comprising:
a processor; and
a computer-readable storage device containing instructions that when executed by the processor cause the system to:
receive data from plurality of sources;
perform an analysis on the data to identify targets related to cell differentiation of a desired cell type and sequences of guide RNAs corresponding to the targets.

59. The system of claim 58, wherein the identified targets are a minimum number necessary to establish the cell type and the guide RNAs are the minimum number to direct cell differentiation.

60. The system of claim 59, further comprises identifying a temporal sequence of expression of the genes.

61. The system of claim 59, wherein the targets are mammalian genes.

62. The system of claim 61, wherein the mammalian genes correspond to a species selected from mouse, human, and a combination thereof.
